(19) United States
(12) Patent Application Publication
(10) Pub. No.: US 2019/0054186 A1
(43) Pub. Date: Feb. 21, 2019

(54) Magnetic Nanoparticle-Polymer Complexes and uses Thereof

(71) Applicant: The University of North Carolina at Chapel Hill, Chapel Hill, NC (US)

(72) Inventors: Alexander V. Kabanov, Chapel Hill, NC (US); Marina Sokolsky, Chapel Hill, NC (US); Philise N. Williams, Kernersville, NC (US)

(21) Appl. No.: 16/089,830

(22) PCT Filed: Mar. 31, 2017
(86) PCT No.: PCT/US2017/025250

§ 371 Date: Sep. 28, 2018

Related U.S. Application Data
(60) Provisional application No. 62/316,013, filed on Mar. 31, 2016.

(51) Int. Cl. A61K 47/69; A61K 47/59; A61K 41/00; A61P 35/00

(52) U.S. Cl. CPC A61K 47/6929; A61K 47/6907; A61K 47/6923; B82Y 5/00; A61K 41/0052; A61P 35/00; A61K 47/59

(57) ABSTRACT

The present invention relates to magnetic nanoparticles coated with block copolymers. The invention further relates to methods of increasing cellular uptake of magnetic nanoparticles and use of the coated magnetic particles to selectively kill cancer cells, treat cancer, detect cancer, and for biomedical imaging.

### STATEMENT OF PRIORITY

This application claims the benefit of U.S. Provisional Application Ser. No. 62/316,013, filed Mar. 31, 2016, the entire contents of which are incorporated by reference herein.

### FIELD OF THE INVENTION

The present invention relates to magnetic nanoparticles coated with block copolymers. The invention further relates to methods of increasing cellular uptake of magnetic nanoparticles and use of the coated magnetic particles to selectively kill cancer cells, treat cancer, detect cancer, and for biomedical imaging.

### BACKGROUND OF THE INVENTION

The medicines of the future should be dormant on the way to their target but actuated to execute their therapeutic function once they reach the site of their action within the body. Superparamagnetic iron oxide nanoparticles (MNP) can be remotely actuated by externally applied magnetic fields to kill cancer cells Urries, et al., Nanoscale 6:9230 (2014); Ansari et al., Small 10:566-575, 417, (2014); He et al., Pharm. Res. 30:2445 (2013); Yoo et al., Acc. Chem. Res. 44:863 (2011). One of the most studied modes of remote actuation is magnetic hyperthermia, which utilizes the MNP response to alternating current (AC) magnetic fields of relatively high frequencies, on the order of hundreds of kHz. Once exposed to such fields the MNPs generate heat through Neé1 or Brownian relaxation, depending on the MNP and the surrounding media characteristics (Di Corato et al., Biomaterials 35:6400 (2014)). This heat leads to temperature increases causing subsequent damage to the surrounding cells. For example, Creixell et al. have utilized magnetic nanoparticle heaters along with an AC field of 233 kHz to kill cancer cells by raising the intracellular temperature to 43° C. (Creixell et al., ACS Nano. 5:7124 (2011)). However, magnetic hyperthermia is limited due to challenges in synthesizing non-toxic MNPs with sufficiently high specific absorption rates (SAR), in reaching sufficient intracellular MNP concentrations and in restricting heat dissipation from a tumor to adjacent healthy tissues (Di Corato et al., Biomaterials 35:6400 (2014); Andra et al., J Magnetism Magnetic Materials 194:197 (1999); Sonvico et al., Bioconjug. Chem. 16:1181 (2005); Salunkhe et al., Curr. Top. Med. Chem. 14:572 (2014)). Recently the concept of surface heating has attracted increased attention. This concept emphasizes energy dissipation in the absence of measurable bulk heating which results in cell death (Creixell et al., ACS Nano 5:7124 (2011)). For example, in one study, cancer cells were incubated with MNPs conjugated with epidermal growth factor (EGF). The targeted MNP, upon exposure to the AC magnetic field (B=47 mT, f=233 kHz), produced a significant reduction in cell viability compared to the non-targeted particles without a perceptible temperature rise. The same group suggested that exposure of EGF-modified MNPs to AC magnetic fields results in lysosomal permeabilization (LMP) due to localized surface heating (Domenech et al., M., ACS Nano 7:5091 (2013)).

The present invention addresses previous shortcomings in the art by providing polymer-coated MNPs actuated inside the cells by low or super low frequency AC magnetic fields. These particles do not cause significant damage to biological tissues but result in magneto-mechanical actuation of the MNPs and promotion of cancer cell death.

### SUMMARY OF THE INVENTION

The present invention is based, in part, on the development of polymer-coated MNPs with improved uptake into cells. The present invention is further based on the actuation of MNPs in cells by low or super low frequency AC magnetic fields, leading to magneto-mechanical actuation of the MNPs and selective death of cancer cells. Such magnetic fields are not expected to produce heat or cause significant damage to biological tissues.

Accordingly, one aspect of the invention relates to a magnetic nanoparticle particle complex (MNPC) comprising a magnetic nanoparticle coated with one or more polymer. The invention is further based on MNPCs comprising a magnetic nanoparticle coated with a polymer comprising at least one hydrophilic chain. In one further aspect a polymer can be a block copolymer and can comprise at least one hydrophilic block and at least one hydrophobic block. In another aspect polymers can optionally contain a polyelectrolyte chain that is covalently linked to at least one hydrophilic chain or a block copolymer.

A further aspect of the invention relates to a MNPC in which a polymer is attached to the to the magnetic nanoparticle via a polyelectrolyte chain or an anchoring group, and the polyelectrolyte chain or anchoring group is covalently linked to the polymer. Another aspect of the invention relates to a MNPC in which the magnetic nanoparticle is hydrophobically modified and connected non-covalently to a hydrophobic block of a block copolymer. The surface of the magnetic nanoparticles can be optionally modified with hydrophobic moieties and the hydrophobically modified magnetic nanoparticles are linked to the hydrophobic groups of the block copolymer.

In one aspect of the invention the MNPC comprises micelles formed by hydrophilic and hydrophobic blocks of at least one block copolymer. In another aspect the MNPC can incorporate drug molecules via molecular interactions. The drug containing MNPCs are useful for theranostics involving drug delivery, imaging and remotely actuated treatment of the disease.

A further aspect of the invention relates to a method of increasing cellular uptake of a MNP, comprising coating the MNP with one or more block copolymers, thereby increasing cellular uptake of the MNP.

Another aspect of the invention relates to a method of treating cancer in a subject in need thereof, comprising administering to the subject a MNP and remotely actuating the MNP with a low or super low frequency magnetic field, thereby treating the cancer.

An additional aspect of the invention relates to a method of treating cancer in a subject in need thereof, comprising administering to the subject the MNPC of the invention, and remotely actuating the MNPC with a magnetic field, thereby treating the cancer.

A further aspect of the invention relates to a method of selectively killing a cancer cell in the presence of non-cancer cells, comprising delivering to the cancer cell and the non-cancer cells a MNP, and remotely actuating the MNP with a low or super low frequency magnetic field, thereby selectively killing the cancer cell.

Another aspect of the invention relates to a method of selectively killing a cancer cell in the presence of non-cancer cells, comprising delivering to the cancer cell and the non-cancer cells the MNPC of the invention, and remotely actuating the MNPC with a magnetic field, thereby selectively killing the cancer cell.

An additional aspect of the invention relates to a method of disrupting the cytoskeleton of a cancer cell, comprising delivering to the cancer cell a MNP, and remotely actuating the MNP with a low or super low frequency magnetic field, thereby disrupting the cytoskeleton of the cancer cell.

A further aspect of the invention relates to a method of disrupting the cytoskeleton of a cancer cell, comprising delivering to the cancer cell the MNPC of the invention, and remotely actuating the MNPC with a magnetic field, thereby disrupting the cytoskeleton of the cancer cell.

Another aspect of the invention relates to a method of obtaining a biomedical image in a subject in need thereof, comprising delivering to the subject the MNPC of the invention and detecting the MNPC, thereby obtaining a biomedical image.

An additional aspect of the invention relates to a method of detecting cancer in a subject in need thereof, comprising delivering to the subject the MNPC of the invention and detecting the MNPC, thereby detecting cancer in the subject.

Another aspect of the invention relates to the use of a MNP and a low or super low frequency magnetic field to treat cancer.

An additional aspect of the invention relates to the use of a MNPC of the invention and a magnetic field to treat cancer.

A further aspect of the invention relates to the use of a MNP and a low or super low frequency magnetic field to selectively kill a cancer cell in the presence of non-cancer cells.

Another aspect of the invention relates to the use of a MNPC of the invention to selectively kill a cancer cell in the presence of non-cancer cells.

An additional aspect of the invention relates to the use of a MNP and a low or super low frequency magnetic field to disrupt the cytoskeleton of a cancer cell.

A further aspect of the invention relates to the use of a MNPC of the invention to disrupt the cytoskeleton of a cancer cell.

Another aspect of the invention relates to the use of a MNPC of the invention to obtain a biomedical image in a subject in need thereof.

An additional aspect of the invention relates to the use of a MNPC of the invention to detect cancer in a subject in need thereof.

These and other aspects of the invention are set forth in more detail in the description of the invention below.

### BRIEF DESCRIPTION OF THE DRAWINGS

FIGS. 1A-1B show flow cytometry of P85-Atto 647. Cells were exposed to 0.08 μg/mL P85-Atto 647 for 1 hour, washed, trypsinized, and resuspended in PBS with 10% BSA for FACS analysis. 10,000 events were analyzed. (FIG. 1A) % Gated cells shows uptake into 100% of cells exposed to P85. (FIG. 1B) Mean fluorescence shows significant internalization of P85 into both cell lines.

FIGS. 2A-2H show confocal microscopy of internalized P85 in BT474 cells (FIGS. 2A, 2C, 2E, 2G) and MDA-MD-231 cells (FIG. 2B, 2D, 2F, 2H). Cells were incubated with (FIG. 2A, FIG. 2E) Lysotracker Red, (FIG. 2B, FIG. 2F) 40 μg/mL Transferrin-Alexa Fluor® 488, and (FIG. 2C, FIG. 2G) P85-Atto 647 1% (v/v) for 1 hour. Cells were washed and visualized by a Zeiss 510 LSM via the 63× oil immersion lens under live cell conditions. Triple colocalization is shown in the composite photo (FIG. 2D, FIG. 2H) as white punctuate structures.

FIGS. 3A-3B show representative TEM images of (FIG. 3A) PAA-P85 coated MNP and (FIG. 3B) PAA-PEG coated MNP.

FIGS. 4A-4F show characteristics of MNP. Particles were dispersed in solvent, sonicated, filtered at 0.22 um, allowed to stand for 45 minutes, and then measured by DLS. This graph represents three independent experiments. Hydrodynamic diameters of (FIG. 4A) PAA-P85-MNP, (FIG. 4B) PAA-PEG-MNP, and (FIG. 4C) PMA-PEG-MNP; and Polydispersity of (FIG. 4D) PAA-P85-MNP, (FIG. 4E) PAA-PEG-MNP, and (FIG. 4F) PMA-PEG-MNP were determined.

FIG. 5 shows cytotoxicity of polymer-MNPs in the absence of AC magnetic field exposure in MDA-MB-231, BT474 and MCF10A cells. The cells were incubated with increasing concentrations of polymer-MNP complexes for 24 h and washed with acid saline to remove any membrane-bound MNP complexes. Cell viability was assessed by MTT assay 24 hours post incubation.

FIGS. 6A-6B show intracellular uptake of polymer-MNP complexes in MDA-MB-231, BT474 and MCF10A cells. (FIG. 6A) Uptake of the polymer-MNP complexes after incubation with complexes for 1 h or 24 h. (FIG. 6B) Dose dependent uptake of PAA-P85-MNP in all three cell lines (*p<0.05).

FIGS. 7A-7E show intracellular distributions of PAA-P85-MNPs in FIG. 7A) MDA-MB-231 FIG. 7B), BT474 and FIG. 7C) MCF10A cells after 24 hours of incubation with 0.05 or 0.5 mg/ml PAA-P85-MNPs. (FIG. 7D) The quantification of the colocalization of Alexa Fluor®647-PAA-P85-MNPs with lysosomes as determined by ImageJ/Fiji. (p<0.01). Lysosomal encapsulation of MNPs seen in (FIG. 7E) TEM images.

FIGS. 8A-8C show the effect of exposure to 50 Hz AC magnetic fields on cell viability. Cells were incubated with various concentrations of PAA-P85 MNPs for 24 h, washed with acid saline and exposed to the field. Viability of MDA-MB-231 (FIG. 8A), BT474 (FIG. 8B) and MCF10A (FIG. 8C) cells was assessed following exposure to a 50 kA/m, 50 Hz or 100 kA/m, 50 Hz AC magnetic field. For each of the field strengths, two different exposure regimes were used: continuous (30 min) or pulsed (10 min on/5 min off) magnetic field. Data shown are mean±SEM (n=15), p<0.05, n.s.=not significant.

FIG. 9 shows intracellular distribution of the PAA-P85-MNP in MDA-MB-231, BT474 and MCF10A cells before and after field exposure. Cells were incubated with Alexa Fluor® 647-PAA-P85-MNP for 24 h at 37° C., washed with acid saline, incubated with Lysotracker™ Green (Alexa® 488) for 1 h and exposed to a 50 kA/m, 50 Hz pulsed (10 min on/5 min off) AC magnetic field. Co-localization of the MNPs with the Lysotracker™ indicated lysosomal uptake. This figure also shows lack of lysosomal membrane permeabilization (LMP) after field exposure. The positive control (cells exposed to hydrogen peroxide) indicates Lysotracker™ staining after LMP. Scale bar=20 μm.

FIG. 10 shows LMP detection using acridine orange in MNP-treated MDA-MB-231, BT474 and MCF10A cells before and after pulsed field exposure. Cells were incubated with PAA-P85-MNP for 24 h at 37° C., washed and exposed to the 50 Hz pulsed AC magnetic field (50 kA/m). After three hours, cells were incubated with 10 μg/mL acridine orange for 15 min. Positive control cells were treated with 150 μM hydrogen peroxide for three hours. The cells exposed to hydrogen peroxide exhibit loss of punctuate red fluorescence while negative controls and cells treated with MNPs do not.

FIG. 11 shows the schematic representation of MNP uptake into lysosomes followed by mechanical movement of the lysosomes to generate forces leading to cytoskeletal disruption.

FIGS. 12A-12C show representative confocal images of actin of the MDA-MB-231, BT474 and nontumorigenic MCF10A cell before and after exposure to a pulsed AC magnetic field with or without treatment with CD and/or PAA-P85-MNP. Insets show a large image of the actin cytoskeleton of a dead (FIG. 12A) MDA-MB-231, (FIG. 12B) BT474 and (FIG. 12C) MCF10A. The graph shows corresponding cell viability for the same conditions in the three cell lines.

FIG. 13 shows confocal microscopy of MDA-MB-231 treated for 24 hours with 0.05 mg/mL AlexaFluor® 647-PAA-P85-MNP. This z-stack shows that the intracellular distribution of MNPs increases towards the basal part of the cell. Quantification of this fluorescence is seen in the graph.

FIG. 14 shows confocal microscopy of BT474 cells treated for 24 h with 0.05 mg/mL AlexaFluor® 647-PAA-P85-MNP. This z-stack shows that the intracellular distribution of MNPs increases towards the basal part of the cell. Quantification of this fluorescence is seen in the graph.

FIGS. 15A-15B show representative TEM images of MCF7 cells treated with PAA-P85-MNPs. (FIG. 15A) shows the association of the MNPs with the cytoskeleton of the cells with (FIG. 15B) showing higher magnification.

FIG. 16 shows results of flow cytometry assay 24 hours after pulsed field exposure. The controls of field and MNPs only show little death. In contrast, the MDA-MB-231 and BT474 show high cell amounts of late stage apoptosis and necrosis after exposure to MNPs and the pulsed field. The MCF10As remain unaffected by MNP and pulsed field exposure.

FIGS. 17A-17C show (FIG. 17A) the structure and characteristics of amphiphilic tri-block poly(2-oxazoline) used in this study, (FIG. 17B) TEM image of uncoated MNPs, and (FIG. 17C) the particle sizes and particle size distribution of uncoated MNPs as measured by TEM. PDI in (FIG. 17A) defines polymer polydispersity index (M_{w}/Mₙ). The results demonstrate formation of MNPs.

FIGS. 18A-18C show (FIG. 18A) stoichiometric composition plot of 6 different PTX-MNPCs, comprising poly(2-oxozaline) (POx), (FIG. 18B) a schematic representation of the process of the preparation of PTX-MNPCs, and (FIG. 18C) the scheme of the synthesis of poly(2-oxazoline)-DSS-dopamine copolymer, containing dopamine anchor group for attachment to the MNPs surface.

FIGS. 19A-19B show the NMR spectra of (FIG. 19A) poly(2-oxazoline) and (FIG. 19B) poly(2-oxazoline)-DSS-dopamine. The peak at 2.5 ppm is the solvent (DMSO) peak. The results demonstrate successful chemical conjugation of the dopamine group to the polymer.

FIGS. 20A-20B show (FIG. 20A) effect of dopamine conjugation to poly(2-oxazoline) on the particle size and PDI of the polymeric micelles in DI water, and (FIG. 20B) LC of the polymeric micelles with respect to PTX, all as functions of the percent of poly(2-oxazoline)-DSS-dopamine blended with unconjugated poly(2-oxazoline) to produce the micelles. The feeding ratio of PTX and polymer was 2:10 (wt:wt). The results suggest that attachment of dopamine to poly(2-oxazoline) chains does not affect the ability of the polymer to self-assemble into the micelles and the ability of the micelles to solubilize drug.

FIGS. 21A-21D show (FIG. 21A) the hydrodynamic sizes (diameters) of PTX-loaded MNPCs dispersed in the DI water and PBS, (FIG. 21B) the zeta potential of PTX-loaded MNPCs, poly(2-oxazoline) polymeric micelles, and poly(2-oxazoline)-DSS-dopamine based polymeric micelles, (FIG. 21C) TEM images of PTX-loaded MNPCs, and (FIG. 21D) magnetization saturation of PTX-loaded MNPCs. Data represent mean±S.D. *p<0.05, **p<0.01.

FIGS. 22A-22E show the effects of AC magnetic field exposures on cell viability. The five different breast cancer cell lines were pre-treated with various doses of MNPCs for 24 h, washed, and then exposed to the AC magnetic field (50 Hz; 50 kA/m). Two different types of AC magnetic field regimes were used: continuous (30 min) or pulsed (10 min field ON/5 min field OFF, for a total of 30 min ON). The following cell lines were studies (FIG. 22A) LCC-6-WT, (FIG. 22B) LCC-6-MDR, (FIG. 22C) MCF-7, (FIG. 22D) BT-474, (FIG. 22E) MDA-MB-231. Data are mean±S.D. (n=6). **p<0.01, ***p<0.001 compared to No exposure. The results suggest that treatment of the cells with MNPCs followed by the field exposure increased toxicity to cancer cells compared to no field treatments, and that the pulsed field has a greater effect than the continuous field exposure.

FIGS. 23A-23C show results of the characterization of the uncoated MNPs and MNP-OA by the SQUID-VSM and TEM. The figures present (FIG. 23A) magnetization saturation of MNP and MNP-OA as measured by SQUID-VSM, (FIG. 23B) the TEM particle size distribution of MNPs and MNP-OA, (FIG. 23C) the representative TEM images of uncoated MNPs (left), and MNP-OA (right). The results demonstrate successful coating of the MNPs with the oleic acid and that the coating does not have a detrimental effect on the superparamagnetic properties of the particles.

FIGS. 24A-24E show the results of physicochemical characterization of PTX loaded MNPCs (“Type-B NanoFerrogel” or “Type-B PTX NFG”). The DLS particle size (hydrodynamic diameter) and PDI of the MNPCs in (FIG. 24A) DI water and (FIG. 24B) PBS over time demonstrate that the MNPCs display colloidal stability over at least one day in DI water or at least two days in PBS. (FIG. 24C) Zeta potential of PTX-loaded MNPCs and poly(2-oxazoline) micelles demonstrate that the micelles incorporate into the MNPCs as evident by the decrease of the zeta potential. (FIG. 24D) Magnetization saturation plot of PTX-loaded MNPCs demonstrates that MNPs included in MNPCs retain superparamagnetic properties. (FIG. 24E) Representative TEM images of PTX loaded MNPCs demonstrating that magnetite particles are incorporated in MNPCs.

FIGS. 25A-25D show (FIG. 25A) a cumulative PTX release from PTX-loaded MNPCs at 37° C. in the presence of 40 g/L BSA, (FIG. 25B) a scheme illustrating the design of the experiment with the pulsed AC magnetic field exposure. Arrow indicates that AC magnetic field was applied for 20 min, (FIG. 25C) the effect of this AC magnetic field exposure on the release of the PTX from the PTX-containing MNPCs at 4 h (data are mean±S.D., n=3, ***p<0.001), and (FIG. 25D) the particle size and PDI change before and after application of the AC magnetic field (PTX-loaded MNPCs are dispersed in PBS. Data are mean±S.D., n=3, **p<0.01 compared to No field). The results demonstrate that the treatment of the drug-loaded MNPCs with the pulsed magnetic field increase drug release and induces changes in the particle size polydispersity.

### DETAILED DESCRIPTION OF THE INVENTION

The present invention will now be described in more detail with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of skill in the art to which this invention belongs. The terminology used in the description of the invention herein is for the purpose of describing particular embodiments only and is not intended to be limiting of the invention. All publications, patent applications, patents, patent publications and other references cited herein are incorporated by reference in their entireties for the teachings relevant to the sentence and/or paragraph in which the reference is presented.

Amino acids are represented herein in the manner recommended by the IUPAC-IUB Biochemical Nomenclature Commission, or (for amino acids) by either the one-letter code, or the three letter code, both in accordance with 37 C.F.R. § 1.822 and established usage.

As used in the description of the invention and the appended claims, the singular forms “a,” “an,” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of polypeptide, dose, time, temperature, enzymatic activity or other biological activity and the like, is meant to encompass variations of ±20%, +10%, ±5%, +1%, +0.5%, or even ±0.1% of the specified amount.

The transitional phrase “consisting essentially of” means that the scope of a claim is to be interpreted to encompass the specified materials or steps recited in the claim, “and those that do not materially affect the basic and novel characteristic(s)” of the claimed invention. See, In re Herz, 537 F.2d 549, 551-52, 190 USPQ 461, 463 (CCPA 1976) (emphasis in the original).

The term “modulate,” “modulates,” or “modulation” refers to enhancement (e.g., an increase) or inhibition (e.g., a decrease) in the specified level or activity.

The term “enhance” or “increase” refers to an increase in the specified parameter of at least about 1.25-fold, 1.5-fold, 2-fold, 3-fold, 4-fold, 5-fold, 6-fold, 8-fold, 10-fold, twelve-fold, or even fifteen-fold.

The term “inhibit” or “reduce” or grammatical variations thereof as used herein refers to a decrease or diminishment in the specified level or activity of at least about 15%, 25%, 35%, 40%, 50%, 60%, 75%, 80%, 90%, 95% or more. In particular embodiments, the inhibition or reduction results in little or essentially no detectable activity (at most, an insignificant amount, e.g., less than about 10% or even 5%).

A “therapeutically effective” amount as used herein is an amount that provides some improvement or benefit to the subject. Alternatively stated, a “therapeutically effective” amount is an amount that will provide some alleviation, mitigation, or decrease in at least one clinical symptom in the subject. Those skilled in the art will appreciate that the therapeutic effects need not be complete or curative, as long as some benefit is provided to the subject.

A “diagnostically effective” amount as used herein is an amount that provides or assists in providing a diagnosis of the subject.

By the terms “treat,” “treating,” or “treatment of,” it is intended that the severity of the subject's condition is reduced or at least partially improved or modified and that some alleviation, mitigation or decrease in at least one clinical symptom is achieved.

As used herein, the term “polymer” or “polymer chain” or “polymeric chain”, as used herein interchangeably, refers to a molecule formed by covalent linking of monomeric units. The term “block copolymer,” as used herein, refers to a combination of two or more polymeric chains of constitutionally or configurationally different features linked to each other. Such distinct polymeric chains of block copolymers are termed “blocks”. For example, “block copolymer” refers to conjugates of at least two different polymer segments, wherein each polymer segment comprises two or more adjacent units of the same kind.

The term “amphiphilic block copolymer,” as used herein, refers to a block copolymer comprised of at least one hydrophilic polymeric chain and at least one hydrophobic polymeric chain. Examples of hydrophilic polymeric chains include polyethers (e.g., poly(ethylene oxide) (PEO) (or poly(oxyethylene) that is used interchangeably with poly(ethylene glycol) (PEG)), polysaccharides (e.g., dextran), polyglycerol, homopolymers and copolymers of vinyl monomers (e.g., polyacrylamide, polyacrylic esters (e.g., polyacryloyl morpholine), polymethacrylamide, poly(N-(2-hydroxypropyl)methacrylamide, polyvinyl alcohol, polyvinyl pyrrolidone, polyvinyltriazole, N-oxide of polyvinylpyridine, copolymer of vinylpyridine and vinylpyridine N-oxide) polyortho esters, polyaminoacids, polyglycerols, poly(2-oxazolines) (e.g., poly(2-methyl-2-oxazoline) (PMeOx), poly(2-ethyl-2-oxazoline) (PEtOx) and their copolymers), polysarcosine and their derivatives and the like. Examples of hydrophobic polymeric chains include poly(propylene oxide) (PPO) (or poly(oxypropylene) that is used interchangeably with PPO), copolymers of poly(ethylene oxide) and PEO, polyalkylene oxide other than PEO and PPO, poly(2-oxazolines) (e.g., poly-(2-propyl-2-oxazoline), poly(2-butyl-2-oxazoline), 2-isobutyl-oxazoline, 2-sec-butyl-2-oxazoline, 2-pentyl-2-oxazoline, 2-heptyl-2-oxazoline, 2-benzyl-2-oxazoline, 2-nonyl-2-oxazoline, and the like), polycaprolactone, poly(D,L-lactide), homopolymers and copolymers of hydrophobic amino acids and derivatives of aminoacids (e.g., alanine, valine, isoleucine, leucine, norleucine, phenylalanine, tyrosine, tryptophan, threonine, proline, cistein, methionone, serine, glutamine, aparagine), poly(β-benzyl-L-aspartate) and the like.

The term “magnetic nanoparticle polymer complex” as used herein refers to a complex resulting from the interaction between a magnetic nanoparticle and a polymer. The complexes may or may not be crosslinked after formation to stabilize the complex.

A “low frequency magnetic field” is a magnetic field having a frequency of about 300 Hz to 10 kHz.

A “super low frequency magnetic field” is a magnetic field having a frequency of about 300 Hz or less.

The term “cancer,” as used herein, refers to any benign or malignant abnormal growth of cells. Examples include, without limitation, breast cancer, prostate cancer, lymphoma, skin cancer, pancreatic cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma. In some embodiments, the cancer is selected from the group of tumor-forming cancers.

The term “breast cancer,” as used herein, refers to a cancer that starts in the cells of the breast of a subject. The term includes invasive and in situ cancers, and encompasses all subtypes of breast cancer, including basal subtype (ER negative and Her2/neu negative), Her2/neu subtype (Her2/neu positive and ER negative); and luminal subtype (ER positive).

A first aspect of the invention relates to the development of MNPCs with increased cellular uptake. The MNPCs are useful for any method or technique in which MNPs have previously been used, including therapeutic, diagnostic, and imaging uses.

One aspect of the invention relates to a MNPC comprising a magnetic nanoparticle coated with one or more block copolymers, wherein at least one block copolymer comprises a block of a poloxamer comprising about 2400 molecular weight poly(propylene oxide) and about 50% poly(ethylene oxide).

The magnetic nanoparticle to be coated can be any nanoparticle known in the art, e.g., a superparamagnetic nanoparticle, e.g., a nanoparticle composed of magnetite (Fe₃O₄) or other iron oxides. Such nanoparticles may be prepared by methods known in the art, such as thermal decomposition. In some embodiments, the magnetic nanoparticle has a diameter of less than about 100 nm, e.g., less than about 50 nm, e.g., less than about 10 nm, e.g., about 1, 2, 3, 4, 5, 6, 7, 8, 9, or 10 nm or any range therein.

Smaller particles of 10 nm and less are particularly preferred. Without limiting this invention to a specific theory it is noted that such nanoparticles can be taken up by cancer and other malignant cells and be transported to specific intracellular organelles such as lysosomes, nuclei, mitochondria and the like where they can exert their action on the cells. In one aspect of the embodiment the magnetic nanoparticles are spherical. In another aspect of this invention the magnetic nanoparticles are non-spherical. Such non-spherical nanoparticles have an aspect ratio of about 2, preferably of at least about 3, still more preferred of at least about 5, yet still more preferred of about 10 and more.

In some embodiments, the MNP is coated with 1, 2, 3, 4, or more block copolymers. Block copolymers are conjugates of at least two different polymer segments. The simplest block copolymer architecture contains two segments joined at their termini to give an A-B type diblock. Consequent conjugation of more than two segments by their termini yields A-B-A type triblock, A-B-A-B-type multiblock, or even multisegment A-B-C-architectures. If a main chain in the block copolymer can be defined in which one or several repeating units are linked to different polymer segments, then the copolymer has a graft architecture of, e.g., an A(B)ₙ type. More complex architectures include for example (AB)ₙ or AₙBₘ, starblocks which have more than two polymer segments linked to a single center. An exemplary block copolymer of the instant invention would have the formula A-B or B-A, wherein A is a polyion segment and B is a nonionic water soluble polymer segment. The segments of the block copolymer may have from about 2 to about 1000 repeating units or monomers.

In some embodiments of the instant invention, the MNP is coated by a block copolymer or combination of several block copolymers, such as amphiphilic block copolymers. In a particular embodiment, the amphiphilic block copolymers comprise at least one block of PEO and at least one block of PPO. In a particular embodiment, the amphiphilic block copolymer is a triblock of PEO-PPO-PEO. Polymers comprising at least one block of PEO and at least one block of PPO are commercially available under such generic trade names as “lipoloxamers,” “Pluronic,” “poloxamers,” and “synperonics.” Examples of poloxamers include, without limitation, Pluronic® L31, L35, F38, L42, L43, L44, L61, L62, L63, L64, P65, F68, L72, P75, F77, L81, P84, P85, F87, F88, L92, F98, L101, P103, P104, P105, F108, L121, L122, L123, F127, 10R5, 10R8, 12R3, 17R1, 17R2, 17R4, 17R8, 22R4, 25R1, 25R2, 25R4, 25R5, 25R8, 31R1, 31R2, and 31R4. Pluronic® block copolymers are designated by a letter prefix followed by a two or a three digit number. The letter prefixes (L, P, or F) refer to the physical form of each polymer, (liquid, paste, or flakeable solid). The numeric code defines the structural parameters of the block copolymer. The last digit of this code approximates the total weight content of PEO blocks in tens of weight percent (for example, 80% weight if the digit is 8, or 10% weight if the digit is 1). The remaining first one or two digits encode the molecular mass of the central PPO block. To decipher the code, one should multiply the corresponding number by 300 to obtain the approximate molecular mass in daltons (Da). Therefore Pluronic® nomenclature provides a convenient approach to estimate the characteristics of the block copolymer in the absence of reference literature. For example, the code ‘F127’ defines the block copolymer, which is a solid, has a PO block of approximately 3600 Da (12×300) and 70% weight of EO. The precise molecular characteristics of each Pluronic® block copolymer can be obtained from the manufacturer. Amphiphilic block copolymers such as Pluronic® block copolymers may be characterized by different hydrophilic-lipophilic balance (HLB) (Kozlov et al. (2000) Macromolecules, 33:3305-3313). The HLB value, which typically falls in the range of 1 to 31 for Pluronic® block copolymers, reflects the balance of the size and strength of the hydrophilic groups and lipophilic groups of the polymer (see, for example, Attwood and Florence (1983) “Surfactant Systems: Their Chemistry, Pharmacy and Biology,” Chapman and Hall, New York) and can be determined experimentally by, for example, the phenol titration method of Marszall (see, for example, “Parfumerie, Kosmetik”, Vol. 60, 1979, pp. 444-448; Rompp, Chemistry Lexicon, 8th Edition 1983, p. 1750; U.S. Pat. No. 4,795,643). HLB values for Pluronic® polymers are available from BASF Corp. HLB values can be approximated by the formula:

$$HLB = - 36 \frac{y}{x + y} + 33 ,$$

wherein y is the number of hydrophobic propylene oxide units and x is the number of hydrophilic ethylene oxide units, though HLB values provided by BASF are preferred. Notably, as hydrophobicity increases, HLB decreases. In a particular embodiment, the amphiphilic block copolymer of the instant invention has an intermediate HLB or low HLB. For example, the HLB for the amphiphilic block copolymer useful on this invention may be about 20 or less, particularly about 18 or less, particularly about 16 or less. In some preferred embodiments the HLB for the amphiphilic block copolymer is in the range from 12 to 18. In some embodiments, the molecular mass of the PPO block is between about 300 and about 4000, e.g., between about 800 and about 3600, e.g., between about 1000 and about 2900, e.g., between about 1400 and about 2500. The physical and molecular characteristics of Pluronic® polymers are well known in the art and can be found, for example, in Paschalis et al., Colloids and Surfaces A: Physicochemical and Engineering Aspects 96, 1-46 (1995) and Kozlov et al., Macromolecules 33:3305-3313 (2000), incorporated herein by reference.

In certain embodiments, at least one block copolymer comprises a polyelectrolyte block or polyion block, such as polycation or polyanion block. Preferred polycations include polyamines (e.g., spermine, polyspermine, polyethyleneimine, polypropyleneimine, polybutileneimine, polypentyleneimine, polyhexyleneimine and copolymers thereof), copolymers of tertiary amines and secondary amines, partially or completely quaternized amines, the quaternary ammonium salts of the polycation fragments, polypeptides such as poly-L-lysine, poly-D-lysine, poly-L-arginine, poly-D-arginine and their copolymers, N-substituted polyaspartamides such as poly[N-(2-aminoethyl)aspartamide] [PAsp(EDA)], poly{N—[N′-(2-aminoethyl)-2-aminoethyl]aspartamide [PAsp(DET)], poly(N—{N′—[N″-(2-aminoethyl)-2-aminoethyl]-2-aminoethyl} aspartamide) [PAsp(TET)], poly-[N—(N′-{N″—[N′″-(2-aminoethyl)-2-aminoethyl]-2-aminoethyl}-2-aminoethyl)aspartamide] [PAsp(TEP)], poly(amidoamine)s and the like. Particularly preferred polycation fragments are those having a plurality of cationic repeating units of the formula —N—R0, wherein R0 is a straight chain aliphatic group of 2 to 6 carbon atoms, which may be substituted. Each —NHR0-repeating unit in a polycation can be the same or different from another —NHR0-repeating unit in the fragment. Examples of polyanions include, without limitation, polymers and their salts comprising units deriving from one or more monomers including: unsaturated ethylenic monocarboxylic acids, unsaturated ethylenic dicarboxylic acids, ethylenic monomers comprising a sulfonic acid group, their alkali metal, and their ammonium salts. Examples of these monomers include acrylic acid, methacrylic acid, aspartic acid, alpha-acrylamidomethylpropanesulphonic acid, 2-acrylamido-2-methylpropanesulphonic acid, citrazinic acid, citraconic acid, trans-cinnamic acid, 4-hydroxy cinnamic acid, trans-glutaconic acid, glutamic acid, itaconic acid, fumaric acid, linoleic acid, linolenic acid, maleic acid, nucleic acids, trans-beta-hydromuconic acid, trans-trans-muconic acid, oleic acid, 1,4-phenylenediacrylic acid, phosphate 2-propene-1-sulfonic acid, ricinoleic acid, 4-styrene sulfonic acid, styrenesulphonic acid, 2-sulphoethyl methacrylate, trans-traumatic acid, vinylsulfonic acid, vinylbenzenesulphonic acid, vinyl phosphoric acid, vinylbenzoic acid and vinylglycolic acid and the like as well as carboxylated dextran, sulphonated dextran, heparin and the like. The examples of polyanions include, but are not limited to, polymaleic acid, polyacrylic acid (PAA) and/or polymethacrylic acid (PMA), glycosaminoglycans such as heparin and other anionic polysaccharides, polyamino acids such as poly-L-glutamic acid, poly-D-glutamic acid, poly-L-aspartic acid, poly-D-aspartic acid and their copolymers, and their salts. The polycations and polyanions of the invention can be randomly branched or have a dendrimer architecture. In some embodiments it is preferred that the polyion of this invention is covalently linked to a lipid moiety. In certain embodiments, at least one block copolymer comprises at least one polyacid block and at least one nonionic block. In certain embodiments, the polyelectrolyte block can be chemically linked or conjugated to an amphiphilic block copolymer. In certain embodiments, at least one block copolymer comprises a polyacid block, e.g., polyacrylic acid (PAA) and/or polymethacrylic acid (PMA). In certain embodiments, the at least one block copolymer comprises at least one polyacid block and at least one poloxamer block.

The poloxamer block may comprise any poloxamer known in the art, e.g., a PLURONIC® poloxamer, e.g., PLURONIC® P85. In some embodiments, the poloxamer comprises about 2400 g/mol molecular mass poly(propylene oxide) and about 50% poly(ethylene oxide) content. In some embodiments, the poloxamer comprises poly(ethylene oxide)₂₀₋₃₀-b-poly(propylene oxide)₃₅₋₄₅-b-poly(ethylene oxide)₂₀₋₃₀ block copolymer, e.g., poly(ethylene oxide)₂₅-b-poly(propylene oxide)₄₀-b-poly(ethylene oxide)₂₅ block copolymer. In some embodiments, the at least one block copolymer is a polyacrylic acid-poloxamer copolymer. In some embodiments, the at least one block copolymer is a PAA-b-P85-b-PAA pentablock copolymer. In certain embodiments, the at least one block copolymer comprising a at least one polyacid block and at least one poloxamer block is the only block copolymer coated in the nanoparticle. In certain embodiments, the at least one block copolymer comprising a at least one polyacid block and at least one poloxamer block is one of two different block copolymer coated in the nanoparticle.

In some proffered embodiments, invention relates to a MNPC in which a polymer is attached to the to the magnetic nanoparticle via a polyelectrolyte chain or an anchor group, and this polyelectrolyte chain or anchor group is covalently linked to this polymer. Examples of anchor groups useful in this invention include groups that can tightly bind to MNP surface, including but not limited to, dihydroxyphenols, such as dophamine, 3,4-dihydroxy-L-phenylalanine (L-DOPA), 3′,4,-dihydroxy-2 (methylamino)acetophenone, trihydroxyphenols and other polyhydroxylphenols, phosphonates such as bisphosphonate, alendronate, iminodi(methylphosphonic acid), N-(phosphonomethyl)glycine, carboxylic acids and their derivatives such as γ-aminobutyric acid, trivinylsiloxy-group modified with mercaptoacetic acid or mercaptosuccinic acid and the like, quaternary amines and ammonium salts, etc.

In some preferred embodiments of the invention the MNPC comprise micelles formed by hydrophilic and hydrophobic blocks of at least one block copolymer. A micelle, as referred to herein, is generally an aggregate of amphiphilic copolymers presenting a hydrophilic corona formed by the hydrophilic parts of the copolymer and sequestering the hydrophobic parts of said amphiphilic copolymers in the interior of the micelle. Particularly suitable copolymers for the formation of micelles are the block copolymers discussed above as a preferred embodiment of the copolymers. Micelles according to the invention are three-dimensional entities. Generally, micelles are formed when the concentration of the constituent amphiphilic molecules in an aqueous solution exceeds a certain value. This, value is referred to as the critical micelle concentration (CMC), which may be determined by using a fluorescent probe, such as pyrene, which partitions into the hydrophobic core of the micelles formed above the CMC value. More specifically, micelles according to the invention form, for example, by self-aggregation of the amphiphilic block copolymers in hydrophilic, preferably aqueous solutions. Upon formation of the micelles, the hydrophilic regions of said amphiphilic copolymers are in contact with the surrounding solvent, whereas the hydrophobic regions are facing towards the center of the micelle. In the context of the invention, the center of a micelle typically incorporates the hydrophobic active agent. A micelle may also be referred to as a “polymeric nanoparticle” because of its size in the nanometer range and its constituents being of polymeric nature. Aggregates, particularly micelles of variable size, may be formed by the pharmaceutical compositions according to the invention, depending on factors such as the molecular weight of the copolymer used or the drug load. Generally preferred are aggregates or micelles within a size range of about 5-500 nm, more preferably between about 5 and 100 nm. However, it is possible to advantageously form aggregates or micelles with sizes ranging from about 5 to 100 or about 10 to 50 nm or even from about 10 to 30 nm, as determined by dynamic light scattering (DLS), which are particularly suitable for intravenous administration. Advantageously, the micelles typically have narrow particle size distributions (DLS polydispersity index (PDI)≤0.2 or even PDI≤0.1, unless indicated otherwise. PDI defines polydispersity index determined by DLS). Typically, the aggregates, particularly micelles, form in water or aqueous media. Thus, the aggregates, particularly micelles, of a composition according to the invention, may be formed, e.g., by the thin film dissolution method. In this method, the copolymer and the active agent are dissolved in a common solvent, such as acetonitrile or dimethylsulfoxide. After removal of the solvent (e.g., by a stream of inert gas, gentle heating and/or application of reduced pressure), films formed by the polymer and the active agent can be easily dissolved in water or aqueous solutions and may be tempered at increased temperatures. When the films are dissolved, the aggregates, preferably micelles, form. The stability of the aggregates allows the resulting solutions to be dried to form a powder. For example, they can be freeze-dried, generally without the need for a cryoprotectant, and reconstituted in water or aqueous solutions without compromising loading capacities, particle integrity or particle sizes.

In some embodiments MNPC contain at least two distinct structural domains—a magnetite MNP domain and a polymeric micelle or polyion complex domain connected with each other. To manufacture such MNPCs the surface of the MNP can be grafted with block copolymers having a hydrophilic block and at least one of the hydrophobic or polyelectrolyte blocks. In aqueous media these materials spontaneously form MNPC due to aggregation of hydrophobic blocks. The resulting polymeric micelle domains can additionally incorporate hydrophobic solutes. Self-assembly of polyelectrolyte containing materials can be induced by adding an oppositely charged amphiphile, or charged therapeutic agents or polyelectrolyte that will form a polyion complex with the polyelectrolyte blocks. In some cases to prepare MNPC the MNP are reacted with amphiphilic block copolymers, for example, ABA copolymers, where A represents the hydrophilic (spacer) block, and B represents the hydrophobic (functional) block. The A block adjacent to the anchor group serve as a tether and the B blocks can self-assemble into aggregates/surface-bound micelles. The second A block ensures that the hybrid-solvent interface is covered with non-ionic hydrophilic polymer. MNPC can be also produced in the organic solvent or in the aqueous media by reacting MNP dispersed in aqueous solution with the micelles comprising at least one type of an amphiphilic block copolymer in which this amphiphilic copolymer contains polyelectrolyte or anchor groups. MNPC can comprise single MNP “cores” or small clusters of MNP covered with the block copolymer micelles or clusters of multiple MNP interconnected with the block copolymer micelles. Without limiting this invention to a specific theory, the self-assembly behavior and structures formed strongly depend on the density of the block copolymer chains grafted onto the MNP surface. Therefore, the grafting density is varied for each copolymer type to obtain the desired parameters of MNPC in particular desired particle size as defined herein. In other examples, the AB diblock copolymers are used to coat the MNPs. Like in the previous case these block copolymers are attached to the MNP surface through the anchor group(s) located in the hydrophilic A block. The hydrophobic B block in this design will face the organic solvent. Upon transfer of such materials to aqueous media different aggregates can form in a concentration-dependent fashion. At low concentrations, isolated coated MNPs resemble flower-like micelles. At higher concentrations, particles can crosslink through hydrophobic interactions of the B blocks. Selected materials will exhibit CMC-like behavior, which can be characterized using DLS, tensiometry, viscosimetry and fluorescent probes (such as pyrene for CMC determination). The resulting materials swell in water due to the presence of hydrophilic A blocks, and form nano-ferrogel dispersions with multiple MNPs linked to each other through block copolymer micelles.

The stability, concentration dependence, and dimensions of such aggregates depend strongly on the nature of the hydrophobic B-block, its molar mass (degree of polymerization) and density of coating. Specifically, B-blocks forming crystalline structures or those with high Tg will also likely result in more stable aggregates, with low CMC. Altogether, the aggregation behavior and colloidal stability of the resulting materials strongly depend on the overall material design, and especially the structure of the coating block copolymers. In all designs the MNP-coated block copolymers can be blended with amphiphilic AB, or ABA block copolymers without anchor groups to improve the dispersion stability of such materials.

In some embodiments, the invention relates to MNPC in which the magnetic nanoparticle is hydrophobically modified and connected non-covalently to a hydrophobic block of a block copolymer. The surface of the magnetic nanoparticles can be optionally modified with hydrophobic moieties and the modified MNP are linked to the hydrophobic groups of the block copolymer. For instance, such MNP modified with hydrophobic moieties can be solubilized in block copolymer micelles coming in contact with the hydrophobic blocks of the block copolymer molecules comprising these micelles. Examples of hydrophobic moieties useful to modify the surface include but are not limited to fatty acids (such as lauric acid, linoleic acid, oleic acid, palmitic acid, stearic acid), and the like (see Cano, M., Sbargoud, K., Allard, E., & Larpent, C. Magnetic separation of fatty acids with iron oxide nanoparticles and application to extractive deacidification of vegetable oils. Green Chemistry, 2012m 14(6), 1786-1795; Zhang, L., He, R., & Gu, H. C. Oleic acid coating on the monodisperse magnetite nanoparticles. Applied Surface Science, 2006, 253(5), 2611-2617; Sahoo, Y., Pizem, H., Fried, T., Golodnitsky, D., Burstein, L., Sukenik, C. N., & Markovich, G. Alkyl phosphonate/phosphate coating on magnetite nanoparticles: a comparison with fatty acids. Langmuir, 2001, 17(25), 7907-7911). The hydrophobic moiety can be a surfactant. Cationic surfactants suitable for use in the present compositions include primary amines (e.g., hexylamine, heptylamine, octylamine, decylamine, undecylamine, dodecylamine, pentadecyl amine, hexadecyl amine, oleylamine, stearylamine, diaminopropane, diaminobutane, diaminopentane, diaminohexane, diaminoheptane, diaminooctane, diaminononane, diaminodecane, diaminododecane), secondary amines (e.g., N,N-distearylamine), tertiary amines (e.g., N,N′,N′-polyoxyethylene(10)-N-tallow-1,3-diaminopropane), alkyl trimethyl quaternary ammonium salts, dialkyldimethyl quaternary ammonium salts, ethoxylated quaternary salts (Ethoquads), e.g., dodecyltrimethylammonium bromide, hexadecyltrimethylammonium bromide, alkyltrimethylammonium bromide, tetradecyltrimethylammonium bromide, oleyltrimethylammonium chloride, benzalkonium chloride, cetyldimethylethylammonium bromide, dimethyldioctadecyl ammonium bromide, methylbenzethonium chloride, decamethonium chloride, methyl mixed trialkyl ammonium chloride, methyl trioctylammonium chloride, 1,2-diacyl-3-(trimethylammonio)propane (acyl group=dimyristoyl, dipalmitoyl, distearoyl, dioleoyl), 1,2-diacyl-3-(dimethylammonio)propane (acyl group=dimyristoyl, dipalmitoyl, distearoyl, dioleoyl), 1,2-dioleoyl-3-(4′-trimethylammonio) butanoyl-sn-glycerol, 1,2-dioleoyl-3-succinyl-sn-glycerol choline ester, cholesteryl (4′-trimethylammonio) butanoate), N-alkyl pyridinium and quinaldinium salts (e.g., cetylpyridinium halide, N-alkylpiperidinium salts, dialkyldimetylammonium salts, dicationic bolaform electrolytes (C₁₂Me₆; C₁₂Bu₆), dialkylglycerylphosphorylcholine, lysolecithin), cholesterol hemisuccinate choline ester, lipopolyamines, e.g., dioctadecylamidoglycylspermine (DOGS), dipalmitoyl phosphatidylethanolamidospermine (DPPES), N′-octadecyl-sperminecarboxamide hydroxytrifluoroacetate, N′,N″-dioctadecylsperminecarboxamide hydroxytrifluoroacetate, N′-nonafluoropentadecylosperminecarboxamide hydroxytrifluoroacetate, N′,N″-dioctyl(sperminecarbonyl)glycinamide hydroxytrifluoroacetate, N′-(heptadecafluorodecyl)-N′-(nonafluoropentadecyl)-sperminecarbonyl)glycinamedehydroxytrifluoroacetate, N′-[3,6,9-trioxa-7-(2′-oxaeicos-11′-enyl)heptaeicos-18-enyl]-sperminecarboxamide hydroxy-trifluoroacetate, N′-(1,2-dioleoyl-sn-glycero-3-phosphoethanoyl)spermine carboxamide hydroxytrifluoroacetate), 2,3-dioleyloxy-N-[2(spermine-carboxamido)ethyl]-N,N-dimethyl-1-propanamini umtrifluoroacetate (DOSPA), N,N^{I},N^{II},N^{III}-tetramethyl-N,N^{I},N^{II},N^{III}-tetrapalmitylspermine (TM-TPS), N-[1-(2,3-dioleyloxy)propyl]-N,N,N-trimethylamonium chloride (DOTMA), dimethyl dioctadecylammonium bromide (DDAB), 1,2-dioleoyl-3-dimethyl-hydroxyethyl ammonium bromide (DORI), 1,2-dioleyloxypropyl-3-dimethyl-hydroxyethyl ammonium bromide (DORIE), 1,2-dioleyloxypropyl-3-dimethyl-hydroxypropyl ammonium bromide (DORIE-HP), 1,2-dioleyloxypropyl-3-dimethyl-hydroxybutyl ammonium bromide (DORIE-HB), 1,2-dioleyloxypropyl-3-dimethyl-hydroxypentyl ammonium bromide (DORIE-HPe), 1,2-dimyristyloxypropyl-3-dimethyl-hydroxyethyl ammonium bromide (DMRIE), 1,2-dipalmitoyloxypropyl-3-dimethyl-hydroxyethyl ammonium bromide (DPRIE), 1,2-distearoyloxypropyl-3-dimethyl-hydroxyethyl ammonium bromide (DSRIE), N,N-dimethyl-N-[2-(2-methyl-4-(1,1,3,3-tetramethylbutyl)-phenoxy]ethoxy)ethyl]-benzenemethanaminium chloride (DEBDA), N-[1-(2,3-dioleyloxy)propyl]-N,N,N,-trimethylammonium methylsulfate (DOTAB), 9-(N′,N″-dioctadecylglycinamido)acridine, ethyl 4-[[N-[3-bis(octadecylcarbamoyl)-2-oxapropylcarbonyl] glycinamido]pyrrole-2-carboxamido]-4-pyrrole-2-carboxylate, N′,N′-dioctadecylornithylglycinamide hydroptrifluoroacetate, cationic derivatives of cholesterol (e.g., cholesteryl-3β-oxysuccinamidoethylenetrimethylammonium salt, cholesteryl-3 β-oxy-succinamidoethylenedimethylamine, cholesteryl-3 β-carboxyamidoethylenetrimethyl-ammonium salt, cholesteryl-3 β-carboxyamidoethylenedimethylamine, 3β[N—(N′,N′-dimethylaminoetane-carbomoyl]cholesterol), pH-sensitive cationic lipids (e.g., 4-(2,3-bis-palmitoyloxy-propyl)-1-methyl-1H-imidazole, 4-(2,3-bis-oleoyloxy-propyl)-1-methyl-1H-imidazole, cholesterol-(3-imidazol-1-yl propyl) carbamate, 2,3-bis-palmitoyl-propyl-pyridin-4-yl-amine) and the like. Suitable anionic surfactants for use in the present compositions include alkyl sulfates, alkyl sulfonates, fatty acid soap including salts of saturated and unsaturated fatty acids and derivatives (e.g., arachidonic acid, 5,6-dehydroarachidonic acid, 20-hydroxyarachidonic acid, 20-trifluoro arachidonic acid, docosahexaenoic acid, docosapentaenoic acid, docosatrienoic acid, eicosadienoic acid, 7,7-dimethyl-5,8-eicosadienoic acid, 7,7-dimethyl-5,8-eicosadienoic acid, 8,11-eicosadiynoic acid, eicosapentaenoic acid, eicosatetraynoic acid, eicosatrienoic acid, eicosatriynoic acid, eladic acid, isolinoleic acid, linoelaidic acid, linoleic acid, linolenic acid, dihomo-γ-linolenic acid, γ-linolenic acid, 17-octadecynoic acid, oleic acid, phytanic acid, stearidonic acid, 2-octenoic acid, octanoic acid, nonanoic acid, decanoic acid, undecanoic acid, undecelenic acid, lauric acid, myristoleic acid, myristic acid, palmitic acid, palmitoleic acid, heptadecanoic acid, stearic acid, nonanedecanoic acid, heneicosanoic acid, docasanoic acid, tricosanoic acid, tetracosanoic acid, cis-15-tetracosenoic acid, hexacosanoic acid, heptacosanoic acid, octacosanoic acid, triocantanoic acid), salts of hydroxy-, hydroperoxy-, polyhydroxy-, epoxy-fatty acids, salts of carboxylic acids (e.g., valeric acid, trans-2,4-pentadienoic acid, hexanoic acid, trans-2-hexenoic acid, trans-3-hexenoic acid, 2,6-heptadienoic acid, 6-heptenoic acid, heptanoic acid, pimelic acid, suberic acid, sebacicic acid, azelaic acid, undecanedioic acid, decanedicarboxylic acid, undecanedicarboxylic acid, dodecanedicarboxylic acid, hexadecanedioic acid, docasenedioic acid, tetracosanedioic acid, agaricic acid, aleuritic acid, azafrin, bendazac, benfurodil hemisuccinate, benzylpenicillinic acid, p-(benzylsulfonamido)benzoic acid, biliverdine, bongkrekic acid, bumadizon, caffeic acid, calcium 2-ethylbutanoate, capobenic acid, carprofen, cefodizime, cefmenoxime, cefixime, cefazedone, cefatrizine, cefamandole, cefoperazone, ceforanide, cefotaxime, cefotetan, cefonicid, cefotiam, cefoxitin, cephamycins, cetiridine, cetraric acid, cetraxate, chaulmoorgic acid, chlorambucil, indomethacin, protoporphyrin IX, protizinic acid), prostanoic acid and its derivatives (e.g., prostaglandins), alkyl phosphates, O-phosphates (e.g., benfotiamine), alkyl phosphonates, natural and synthetic lipids (e.g., dimethylallyl pyrophosphate ammonium salt, S-famesylthioacetic acid, famesyl pyrophosphate, 2-hydroxymyristic acid, 2-fluorpalmitic acid, inositoltrphosphates, geranyl pyrophosphate, geranygeranyl pyrophosphate, α-hydroxyfamesyl phosphonic acid, isopentyl pyrophoshate, phosphatidylserines, cardiolipines, phosphatidic acid and derivatives, lysophosphatidic acids, sphingolipids and like), synthetic analogs of lipids such as sodium-dialkyl sulfosuccinate (e.g., Aerosol OT®), n-alkyl ethoxylated sulfates, n-alkyl monothiocarbonates, alkyl- and arylsulfates (asaprol, azosulfamide, p-(benzylsulfonamideo)benzoic acid, cefonicid, CHAPS), mono- and dialkyl dithiophosphates, N-alkanoyl-N-methylglucamine, perfluoroalcanoate, cholate and desoxycholate salts of bile acids, 4-chloroindoleacetic acid, cucurbic acid, jasmonic acid, 7-epi jasmonic acid, 12-oxo phytodienoic acid, traumatic acid, tuberonic acid, abscisic acid, acitertin, and the like. Preferred cationic and anionic surfactants also include fluorocarbon and mixed fluorocarbon-hydrocarbon surfactants. Suitable surfactants include salts of perfluorocarboxylic acids (e.g., pentafluoropropionic acid, heptafluorobutyric acid, nonanfluoropentanoic acid, tridecafluoroheptanoic acid, pentadecafluorooctanoic acid, heptadecafluorononanoic acid, nonadecafluorodecanoic acid, perfluorododecanoic acid, perfluorotetradecanoic acid, hexafluoroglutaric acid, perfluoroadipic acid, perfluorosuberic acid, perfluorosebacicic acid), double tail hybrid surfactants (CₘF₂ₘ₊₁)(CₙH₂ₙ₊₁)CH—OSO₃Na, fluoroaliphatic phosphonates, fluoroaliphatic sulphates, and the like. Surfactants containing strong anions are preferred.

In some embodiments the MNPC can further incorporate therapeutic agent molecules. The drug containing MNPC are useful for theranostics involving drug delivery, imaging and remotely actuated treatment of the disease. Preferably, the therapeutic agent is hydrophobic. Therapeutic agents that may be solubilized or dispersed by the polymers of the present invention can be any bioactive agent and particularly those having limited solubility or dispersibility in an aqueous or hydrophilic environment, or any bioactive agent that requires enhanced solubility or dispersibility. In a particular embodiment, the polymers of the instant invention may be utilized to solubilize highly hydrophobic bioactive substances having a solubility of <1 mg/mL, <0.1 mg/mL, <50 μg/ml, or <10 μg/mL in water or aqueous media in a pH range of 0-14, preferably between pH 4 and 10. Suitable drugs include, without limitation, those presented in Goodman and Gilman's The Pharmacological Basis of Therapeutics (9th Ed.) or The Merck Index (12th Ed.). Genera of drugs include, without limitation, drugs acting at synaptic and neuroeffector junctional sites, drugs acting on the central nervous system, drugs that influence inflammatory responses, drugs that affect the composition of body fluids, drugs affecting renal function and electrolyte metabolism, cardiovascular drugs, drugs affecting gastrointestinal function, drugs affecting uterine motility, chemotherapeutic agents for parasitic infections, chemotherapeutic agents for microbial diseases, antineoplastic agents, immunosuppressive agents, drugs affecting the blood and blood-forming organs, hormones and hormone antagonists, dermatological agents, heavy metal antagonists, vitamins and nutrients, vaccines, oligonucleotides and gene therapies. Examples of therapeutic agents suitable for use in the present invention include, without limitation, protease inhibitors such as atazanavir (ATV) or atazanavir sulfate (ATV sulfate), non-nucleoside reverse transcriptase inhibitor efavirenz (EFV), ATM (Ataxia telangiectasia mutated) kinase inhibitor KU55933, cytoskeletal drugs that target tubulin-paclitaxel (PTX) and docetaxel (DTX), larotaxel, ortataxel, tesetaxel and other taxanes, ATM/ATR (ataxia telangiectasia and Rad3-related protein) inhibitors VE-821 and VE-822, Bcl-2 family protein inhibitors ABT-263 (Navitoclax), ABT-737 and sabutoclax, PI3K (phosphoinositide 3-kinase) inhibitors NVP-BEZ235 and wortmannin, PI3K/AKT (Protein kinase B) inhibitors AZD5363 and LY294002 and LY294002 HCl, check point inhibitor AZD7762, Mtor (mechanistic target of rapamycin) inhibitor AZD8055, alkylating agent cisplatin prodrugs, topoisomerase II inhibitor etoposide (ETO) or VP-16, immune response modifier imiquimod, proteasome inhibitor LDN-57444, TGF beta inhibitors LY2109761 and LY364947, PARP (poly ADP ribose polymerase) inhibitor olaparib (also known as AZD2281 or Ku-0059436), lactone antibiotic brefeldin, and sonic hedgehog inhibitor Vismodegib. Other examples of therapeutic agents include testosterone, testosterone enanthate, testosterone cypionate, methyltestosterone, amphotericin B, nifedipine, griseofulvin, anthracycline antibiotics such as doxorubicin and daunomycin, indomethacin, ibuprofen, etoposide and cyclosporin A. The presence of the polymers in MNPC increases the solubility in water and aqueous solutions by orders of magnitude. This allows for largely increased dose administration to patients and would be particularly beneficial in the treatment of various diseases such as cancer.

In some embodiments, the MNPC including MNP with the coating has a diameter of less than about 200 nm, e.g., less than about 100 nm, e.g., about, 50, 60, 70, 80, 90, or 100 nm or any range therein.

In some embodiments, the MNPC of the invention may further comprise an additional agent which is covalently or non-covalently attached to the MNPC. The additional agent may be, without limitation, a therapeutic agent (e.g., a chemotherapeutic agent), a contrast agent, a targeting moiety (e.g., a cancer cell targeting moiety), or any combination thereof. Various targeting moieties known in the art, such as antibodies, aptamers, peptides, and polysaccharides that can bind a receptor at the surface of tumor cells, can be used in this invention.

The present invention further relates to a method of increasing cellular uptake of a MNP, comprising coating the MNP with one or more block copolymers, thereby increasing cellular uptake of the MNP. The one or more block copolymers may be any of the block copolymers or combinations of block copolymers described above.

One aspect of the invention relates to the use of the MNPCs of the invention in methods for which MNPs are known to be useful, including, without limitation, therapeutic, diagnostic, and biomedical imaging uses.

In one aspect the invention relates to a method of treating cancer in a subject in need thereof, comprising administering to the subject the MNPC of the invention, and remotely actuating the MNPC with a magnetic field, thereby treating the cancer. The magnetic field may be any type of magnetic field known to be useful for actuating MNPs. In some embodiments, the magnetic field is a low or super low frequency magnetic field as discussed further below. A subject in need of cancer treatment is a subject that has been diagnosed with cancer or is suspected of having cancer.

In a further aspect, the invention relates to a method of selectively killing a cancer cell in the presence of non-cancer cells, comprising delivering to the cancer cell and the non-cancer cells the MNPC of the invention, and remotely actuating the MNPC with a magnetic field, thereby selectively killing the cancer cell. In some embodiments, the magnetic field is a low or super low frequency magnetic field as discussed further below. In this aspect the invention relates to a novel cancer therapy approach, in which cancer cells and other cells of the tumor microenvironment are destroyed without use of chemotherapeutic drugs by mechanical motion of magnetic nanoparticles actuated remotely by applied alternating current magnetic fields of very low frequency. Such fields and treatments are safe for surrounding tissues but disrupt the cytoskeleton and kill cancer cells while leaving healthy cells intact. In this aspect of the invention the MNPCs comprising MNPs attached to hydrophilic polyelectrolytes (e.g., polyanion) or hydrophilic non-ionic polymers, such as PEO, PMeOx, PetOx, polysarcosine, and the like, or amphiphilic block copolymers, especially those attached to MNPs via their hydrophilic chains, are preferred.

In another aspect, the invention relates to a method of disrupting the cytoskeleton of a cancer cell, comprising delivering to the cancer cell the MNPC of the invention, and remotely actuating the MNPC with a magnetic field, thereby disrupting the cytoskeleton of the cancer cell. The term “disrupting the cytoskeleton” refers to a breaking down of the cytoskeleton such that at least one activity or function of the cytoskeleton is no longer operative. In some embodiments, the magnetic field is a low or super low frequency magnetic field as discussed further below.

In an additional aspect, the invention relates to a method of obtaining a biomedical image in a subject in need thereof, comprising delivering to the subject the MNPC of the invention and detecting the MNPC, thereby obtaining a biomedical image.

In a further aspect, the invention relates to a method of detecting cancer in a subject in need thereof, comprising delivering to the subject the MNPC of the invention and detecting the MNPC, thereby detecting cancer in the subject.

In each of the above methods, the steps may be carried out as known in the art. Each method is enhanced by virtue of the increased cellular uptake of the MNPCs of the invention, increasing the number of MNPCs accumulating in each cell and/or the number of cells containing MNPCs.

One aspect of the invention relates to the development of methods of actuating MNPs using low or super low frequency magnetic fields. The use of such magnetic fields leads to magneto-mechanical actuation of the MNPs and selective death of cancer cells, without producing heat or causing any damage to biological tissues. The methods of the invention may be an improvement over previous methods of using MNPs both in terms of efficacy and safety.

In one aspect the invention relates to a method of treating cancer in a subject in need thereof, comprising administering to the subject a MNP and remotely actuating the MNP with a low or super low frequency magnetic field, thereby treating the cancer. The MNP may be any MNP known in the art or as described herein. The low or super low frequency magnetic field may have a frequency of about 1 Hz to about 10 kHz or any range therein, e.g., about 5 Hz to about 1 kHz, e.g., about 20 Hz to about 100 Hz, e.g., less than about 250, 200, 150, or 100 Hz. The low or super low frequency magnetic field may have a strength that is less than about 150 kA/m, e.g., less than about 100 kA/m, e.g., less than about 50 kA/m or any range therein.

In some embodiments, the magnetic field may be a constant field administered for a suitable length of time, e.g., about 1 minute to about 120 minutes or more, e.g., about 1, 2, 5, 10, 20, 30, 40, 50, 60, 70, 80, 90, 100, 110, or 120 minutes or any range therein.

In other embodiments, the magnetic field may be a pulsed field administered for a suitable length of time, e.g., about 1 minute to about 120 minutes or more, e.g., about 1, 2, 5, 10, 20, 30, 40, 50, 60, 70, 80, 90, 100, 110, or 120 minutes or any range therein. The pulse pattern may be any suitable pattern, e.g., a pulse of about 1, 2, 5, 10, 20, 30, 40, 50, or 60 minutes interspersed with a non-administration period of about 1, 2, 5, 10, 20, 30, 40, 50, or 60 minutes. The pulse may be repeated as many times as necessary, e.g., about 1, 2, 3, 4, 5, 6, 7, 8, 9, or 10 times. In some embodiments, the pulse pattern is about 1-20 minutes on and about 1-15 minutes off, e.g., about 5-15 minutes on and about 1-10 minutes off, repeated 1, 2, or 3 times. The magnetic field treatment, whether constant or pulsed or a mix thereof, may be repeated more than once a day (e.g., 2-4 times a day), once a day, once a week, once a month, or any other suitable pattern as needed.

In another aspect, the invention relates to a method of selectively killing a cancer cell in the presence of non-cancer cells, comprising delivering to the cancer cell and the non-cancer cells a MNP, and remotely actuating the MNP with a low or super low frequency magnetic field, thereby selectively killing the cancer cell. The MNP may be any MNP known in the art or as described herein. The low or super low frequency magnetic field may be as described above.

In a further aspect, the invention relates to a method of disrupting the cytoskeleton of a cancer cell, comprising delivering to the cancer cell a MNP, and remotely actuating the MNP with a low or super low frequency magnetic field, thereby disrupting the cytoskeleton of the cancer cell. The MNP may be any MNP known in the art or as described herein. The low or super low frequency magnetic field may be as described above.

Without being bound by theory, it is thought that the MNPs upon actuation by a low or super low frequency magnetic field rotate inside the lysosomes in which they accumulate, inducing torques and shear stress on the underlying cytoskeleton. Without being bound to a specific theory it is also thought that the smaller magnetic particles with preferred particle sizes and coatings as defined in this invention can assemble into larger aggregates in the cell organelles, like lysosomes, and being assembled they may move in a synchronized fashion, and their collective motion may increase the stresses exhibited upon intracellular structures. Without being bound to a specific theory it is also thought that the aggregation and stresses may increase upon exposure to a direct current or alternating magnetic field and therefore the effect may further increase when the fields are superimposed and also when at least one field is applied in pulses. The cytoskeleton in cancer cells is more sensitive to mechano-transduction leading to subsequent damage and cell death, whereas induced forces are insufficient to cause damage to the cytoskeleton of non-cancerous cells. This selectivity may be advantageously used in the methods of the present invention.

Another aspect of the invention relates to a kit comprising the MNPCs of the invention and useful for carrying out the methods of the invention. The kit may further comprise additional reagents for carrying out the methods (e.g., buffers, containers, additional therapeutic agents) as well as instructions.

As a further aspect, the invention provides pharmaceutical formulations and methods of administering the same to achieve any of the therapeutic, diagnostic, and imaging effects discussed above. The pharmaceutical formulation may comprise any of the reagents discussed above in a pharmaceutically acceptable carrier.

By “pharmaceutically acceptable” it is meant a material that is not biologically or otherwise undesirable, i.e., the material can be administered to a subject without causing any undesirable biological effects such as toxicity.

The formulations of the invention can optionally comprise medicinal agents, pharmaceutical agents, carriers, adjuvants, dispersing agents, diluents, and the like.

The MNPCs of the invention can be formulated for administration in a pharmaceutical carrier in accordance with known techniques. See, e.g., Remington, The Science And Practice of Pharmacy (9^{th} Ed. 1995). In the manufacture of a pharmaceutical formulation according to the invention, the MNPCs are typically admixed with, inter alia, an acceptable carrier. One or more types of MNPCs can be incorporated in the formulations of the invention, which can be prepared by any of the well-known techniques of pharmacy.

A further aspect of the invention is a method of treating subjects in vivo, comprising administering to a subject a pharmaceutical composition comprising MNPCs of the invention in a pharmaceutically acceptable carrier, wherein the pharmaceutical composition is administered in a therapeutically or diagnostically effective amount. Administration of the MNPCs of the present invention to a human subject or an animal in need thereof can be by any means known in the art for administering compounds.

Non-limiting examples of formulations of the invention include those suitable for oral, rectal, buccal (e.g., sub-lingual), vaginal, parenteral (e.g., subcutaneous, intramuscular including skeletal muscle, cardiac muscle, diaphragm muscle and smooth muscle, intradermal, intravenous, intraperitoneal), topical (i.e., both skin and mucosal surfaces, including airway surfaces), intranasal, transdermal, intraarticular, intracranial, intrathecal, and inhalation administration, administration to the liver by intraportal delivery, as well as direct organ injection (e.g., into the liver, into a limb, into the brain or spinal cord for delivery to the central nervous system, into the pancreas, or into a tumor or the tissue surrounding a tumor). The most suitable route in any given case will depend on the nature and severity of the condition being treated and on the nature of the particular compound which is being used. In some embodiments, it may be desirable to deliver the formulation locally to avoid any side effects associated with systemic administration. For example, local administration can be accomplished by direct injection at the desired treatment site, by introduction intravenously at a site near a desired treatment site (e.g., into a vessel that feeds a treatment site). In some embodiments, the formulation can be delivered locally to ischemic tissue. In certain embodiments, the formulation can be a slow release formulation, e.g., in the form of a slow release depot.

For injection, the carrier will typically be a liquid, such as sterile pyrogen-free water, sterile normal saline, hypertonic saline, pyrogen-free phosphate-buffered saline solution, bacteriostatic water, or Cremophor EL[R] (BASF, Parsippany, N.J.). For other methods of administration, the carrier can be either solid or liquid.

Formulations of the present invention suitable for parenteral administration comprise sterile aqueous and non-aqueous injection solutions of the MNPCs, which preparations are preferably isotonic with the blood of the intended recipient. These preparations can contain anti-oxidants, buffers, bacteriostats and solutes which render the formulation isotonic with the blood of the intended recipient. Aqueous and non-aqueous sterile suspensions can include suspending agents and thickening agents. The formulations can be presented in unit/dose or multi-dose containers, for example sealed ampoules and vials, and can be stored in a freeze-dried (lyophilized) condition requiring only the addition of the sterile liquid carrier, for example, saline or water-for-injection immediately prior to use.

Extemporaneous injection solutions and suspensions can be prepared from sterile powders, granules and tablets of the kind previously described. For example, in one aspect of the present invention, there is provided an injectable, stable, sterile composition comprising MNPCs of the invention, in a unit dosage form in a sealed container. The MNPCs are provided in the form of a lyophilizate which is capable of being reconstituted with a suitable pharmaceutically acceptable carrier to form a liquid composition suitable for injection thereof into a subject. The unit dosage form typically comprises from about 1 mg to about 10 grams of the MNPCs.

Formulations suitable for rectal administration are preferably presented as unit dose suppositories. These can be prepared by admixing the peptide with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which can be used include petroleum jelly, lanoline, polyethylene glycols, alcohols, transdermal enhancers, and combinations of two or more thereof.

Formulations suitable for transdermal administration can be presented as discrete patches adapted to remain in intimate contact with the epidermis of the recipient for a prolonged period of time. Formulations suitable for transdermal administration can also be delivered by iontophoresis (see, for example, Tyle, Pharm. Res. 3:318 (1986)) and typically take the form of an optionally buffered aqueous solution of the peptides. Suitable formulations comprise citrate or bis/tris buffer (pH 6) or ethanol/water and contain from 0.1 to 0.2M of the compound.

The MNPCs can alternatively be formulated for nasal administration or otherwise administered to the lungs of a subject by any suitable means, e.g., administered by an aerosol suspension of respirable particles comprising the MNPCs, which the subject inhales. The respirable particles can be liquid or solid. The term “aerosol” includes any gas-borne suspended phase, which is capable of being inhaled into the bronchioles or nasal passages. Specifically, aerosol includes a gas-borne suspension of droplets, as can be produced in a metered dose inhaler or nebulizer, or in a mist sprayer. Aerosol also includes a dry powder composition suspended in air or other carrier gas, which can be delivered by insufflation from an inhaler device, for example. See Ganderton & Jones, Drug Delivery to the Respiratory Tract, Ellis Horwood (1987); Gonda (1990) Critical Reviews in Therapeutic Drug Carrier Systems 6:273-313; and Raeburn et al., J. Pharmacol. Toxicol. Meth. 27:143 (1992). Aerosols of liquid particles comprising the MNPs can be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the MNPs can likewise be produced with any solid particulate medicament aerosol generator, by techniques known in the pharmaceutical art.

For oral administration, the MNPCs can be administered in solid dosage forms, such as capsules, tablets, and powders, or in liquid dosage forms, such as elixirs, syrups, and suspensions. MNPCs can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate and the like. Examples of additional inactive ingredients that can be added to provide desirable color, taste, stability, buffering capacity, dispersion or other known desirable features are red iron oxide, silica gel, sodium lauryl sulfate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for buccal (sub-lingual) administration include lozenges comprising the MNPCs in a flavored base, usually sucrose and acacia or tragacanth; and pastilles comprising the MNPCs in an inert base such as gelatin and glycerin or sucrose and acacia.

Alternatively, one can administer the MNPCs in a local rather than systemic manner, for example, in a depot or sustained-release formulation.

Further, the present invention provides liposomal formulations of the MNPCs disclosed herein and salts thereof. The technology for forming liposomal suspensions is well known in the art. The liposomal formulations containing the MNPCs can be lyophilized to produce a lyophilizate which can be reconstituted with a pharmaceutically acceptable carrier, such as water, to regenerate a liposomal suspension.

In particular embodiments, the MNPCs are administered to the subject in a therapeutically or diagnostically effective amount, as that term is defined above. Dosages of MNPCs can be determined by methods known in the art, see, e.g., Remington's Pharmaceutical Sciences (Mack Publishing Co., Easton, Pa.). The therapeutically or diagnostically effective dosage of any specific MNPCs will vary somewhat from MNPC to MNPC, and patient to patient, and will depend upon the condition of the patient and the route of delivery. As a general proposition, a dosage from about 0.1 to about 50 mg/kg will have therapeutic or diagnostic efficacy, with all weights being calculated based upon the weight of the MNPCs. Toxicity concerns at the higher level can restrict intravenous dosages to a lower level such as up to about 10 mg/kg, with all weights being calculated based upon the weight of the MNPCs. A dosage from about 10 mg/kg to about 50 mg/kg can be employed for oral administration. Typically, a dosage from about 0.5 mg/kg to 5 mg/kg can be employed for intramuscular injection. Particular dosages are about 1 μmol/kg to 50 μmol/kg, and more particularly to about 22 μmol/kg and to 33 μmol/kg of the MNPCs for intravenous or oral administration, respectively.

In particular embodiments of the invention, more than one administration (e.g., two, three, four, or more administrations) can be employed over a variety of time intervals (e.g., hourly, daily, weekly, monthly, etc.) to achieve therapeutic effects.

The present invention finds use in veterinary and medical applications. Suitable subjects include both avians and mammals, with mammals being preferred. The term “avian” as used herein includes, but is not limited to, chickens, ducks, geese, quail, turkeys, and pheasants. The term “mammal” as used herein includes, but is not limited to, humans, bovines, ovines, caprines, equines, felines, canines, lagomorphs, etc. Human subjects include neonates, infants, juveniles, and adults.

The present invention is more particularly described in the following examples that are intended as illustrative only since numerous modifications and variations therein will be apparent to those skilled in the art.

### Example 1

### Experimental Methods

Cell Lines:

MDA-MB-231 (human triple negative (ER/PR− Her2/neu−) mammary gland adenocarcinoma), BT474 (human breast ductal carcinoma) and MCF10A (human non tumorigenic mammary gland cells) were supplied by ATCC (Manassas, Va.). MDA-MB-231 and BT474 cells were maintained in DMEM (high glucose) containing 10% heat inactivated FBS and 1% penicillin/streptomycin. MCF10A cells were maintained in DME/F12 media containing 10% heat inactivated FBS, 1% penicillin/streptomycin, 10 μL/mL human insulin and 10 ng/mL human epidermal growth factor. All cell cultures were maintained at 37° C. in a 5% CO₂ atmosphere. Human breast cancer cell models were used for this study. MDA-MB-231 human breast cancer cells were initially used to assess the ability of this system to kill a triple negative (ER−/PR−/HER2/neu−) cancer. BT474 human breast ductal carcinoma cells were used to further assess the effects in a cell line with a different cytoskeletal structure. Lastly, MCF10A nontumorigenic human breast cells were used as a control.

Materials:

Lysotracker® Green, TubulinTracker™, Hoechst 33342, Annexin V, propidium iodide, fetal bovine serum (FBS) (both dialyzed and heat inactivated), Dulbecco's Modified Eagle's Medium (DMEM), DMEM:F12, penicillin/streptomycin, human insulin, human epidermal growth factor and Alexa Fluor 647-hydrazine were purchased from Life Technologies (Carlsbad, Calif.). Hydrogen peroxide was purchased from Thermo Fisher Scientific (Waltham, Mass.). Lab-Tek II Chambered Coverglass #1.5 Borosilicate 8 well chambers, used for live cell imaging, were purchased from Fisher Scientific (Waltham, Mass.). High binding strip plates (2×8 MICROLON 96 well) were purchased from Griener Bio-One. MTT reagent (3-(4,5-dimethylthiazol-2-yl)-2,5-diphenyltetrazolium bromide) was purchased from Research Products International (Prospect, Ill.). Cytochalasin D (CD), dimethylsulfoxide (DMSO) and nitric acid (HNO₃) TRACESELECT purity grade, Atto 647 N-hydroxysuccinimide ester, and Sephadex G-50 were purchased from Sigma Aldrich (St. Louis, Mo.). Pluronic® P85 (P85), poly(ethylene oxide)₂₆-b-poly(propylene oxide)₃₉-b-poly(ethylene oxide)₂₆ block copolymer was provided by BASF Corp. (Parsippany, N.J.). All other chemicals were of reagent grade and used without further purification.

AC Magnetic Field Generator:

The super-low frequency AC magnetic field generator was custom designed and purchased from Nanomaterials Ltd. (Tambov, Russia). The unit contains a sinusoidal current generator with variable power (up to 1.5 kW), frequency (in the range from 30 to 3000 Hz) and variable magnetic field amplitude (from 10 to 100 mT). The unit is equipped with a water-cooled inductor with a ferromagnetic core and a temperature-controlled cuvette. The temperature-controlled holder accommodates one 8-well strip plate at a time. The temperature was maintained at 37° C. for all cellular experiments. For all cell experiments, cells were seeded in the middle wells, which were exposed to a homogeneous field. The experiments were conducted at a frequency of 50 Hz and the magnetic field intensity was 50 or 100 kA/m. Field frequency and field intensity were measured and monitored by an oscilloscope throughout the application time.

Synthesis and Characterization of Polymer-MNP Complexes:

The MNP complexes were prepared and coated by ligand exchange with polyanion-PEG or polyanion-P85 block copolymers as described below. The effective hydrodynamic diameter (D_{eff}=intensity averages), polydispersity and ζ-potential of the polymer-MNP complexes were determined by DLS using a Zetasizer Nano ZS (Malvern Instruments Ltd., Malvern, UK). All measurements were performed in automatic mode at 25° C. All measurements were performed at least in triplicate to calculate mean values±standard deviations.

The polymer content in the polymer-MNP complexes was determined by thermogravimetric analysis (Q50, TA Instruments, New Castle, Del.). Approximately 10-15 mg of the samples were loaded and exposed to a heat ramp to 110° C. at a rate of 10° C./min, followed by an isothermal hold for 15 min, and then continued heating to 1000° C. at 10° C./min. Iron content in the polymer-MNP complexes was analyzed by Inductively Coupled Plasma Mass Spectrometry (ICP-MS) (NexION 300D, Perkin Elmer, Waltham, Mass.). Briefly, 0.5 mL of particle solution (1 mg/mL) were mixed with 50 μL of nitric acid and incubated at 70° C. overnight (at least 12 h). Following the digestion, the volume of the solution was adjusted to 1 mL with deionized (DI) water and analyzed by ICP-MS.

Labeling of PAA-P85-MNP with Alexa Fluor®647:

PAA-P85 coated MNP complexes were labeled with the fluorescent dye Alexa-Fluor®647 hydrazine using standard EDC chemistry. Briefly, 4.5 mg of PAA-P85-MNPs were diluted with 0.35 mL of deionized (DI) water and sonicated for 30 minutes followed by addition of 10 mg 1-ethyl-3-(-3-dimethylaminopropyl) carbodiimide hydrochloride (EDC). A stock solution of N-hydroxysulfosuccinimide (S-NHS) (40 mg/mL in DI water) was prepared and 50 μL of this solution was added to the reaction vial. A stock solution of Alexa Fluor®647 hydrazine (1 mg/mL in DI water) was prepared and 0.1 mL was added to the reaction vial. The vial was protected from light and incubated overnight on a shaker at approximately 100 rpm. Alexa Fluor®647-PAA-P85-MNP were purified on a size exclusion column (Sephadex G-50) with phosphate buffered saline (PBS) as the eluent followed by centrifugal filtration with 100 kDa cutoff Centricons (EMD Millipore, Billerica, Mass.). The concentration of MNPs in solution was determined by ICP-MS. Similar to the previously described method, 20 μL of particle solution were mixed with 50 μL of nitric acid and incubated at 70° C. overnight (minimum 12 h). Following the digestion, the volume of the solution was adjusted to 1 mL with DI water and analyzed by ICP-MS.

Cytotoxicity of Polymer-MNP Complexes:

In vitro cytotoxicity of polymer-MNP complexes was assessed in MDA-MB-231, BT474 and MCF10A cells by standard MTT assay as described previously (Ferrari et al., J. Immunol. Meth. 131:165-172 (1990)). Briefly, cells were seeded at 5×10³ cells/well in a 96-well plate and were allowed to adhere for two days. Cells were treated with polymer-MNP complexes at various doses (0.005-0.5 mg/mL polymer-MNP complexes) for 24 h at 37° C., washed with acidic saline (pH 3) to remove non-internalized polymer-MNPs and maintained in complete DMEM for an additional 24 h. All of the samples were tested in triplicate. A standard MTT assay was then performed by addition of 25 μL of MTT dye (5 mg/mL) to each well followed by a 4-h incubation period at 37° C. The resultant formazan was then solubilized in DMSO and absorption was measured at 570 nm using a spectrofluorometer (SpectraMax M5, Molecular Devices Co., USA). The reading taken from the wells with cells cultured with control medium was used as a 100% viability value. The cell viability was calculated as Aₛₐₘₚₗₑ/A_{control}×100%.

Quantitative Uptake of Polymer-MNP Complexes In Vitro:

MDA-MB-231, BT474 and MCF10A cells were seeded at 1×10⁶ cells/well in 6 well plates and allowed to adhere for 3 days. They were then washed and treated with polymer-MNP complexes at various doses (0.005-0.5 mg/mL polymer-MNP complexes) for 1 h or 24 h at 37° C. Cells were rinsed 3 times with acidic saline (pH 3) and harvested using 0.05% trypsin/EDTA. Cells were pelleted, the supernatant was discarded and the cells were resuspended in 0.5 mL of DI water. The cell suspension was then sonicated with a probe sonicator at 10 kHz for 40 s. The cell suspension was digested using nitric acid as previously described. Following the digestion, the volume of the solution was adjusted to 1 mL with DI water and analyzed by ICP-MS.

Intracellular Distributions of PAA-P85-MNPs:

MDA-MB-231, BT474 and MCF10A cells were seeded at 1×10⁵ cells/well in 8-well Lab-Tek II Chamber slides. Cells were allowed to adhere for 3 days and were treated with a specified dosage of Alexa Fluor®647-PAA-P85-MNP for 24 h. After thorough washing, the cells were treated with 100 nM of Lysotracker™ Green (λₑₓ/λₑₘ=504/511 nm) for 1 h and Hoechst 33342 nuclear stain for 15 min. Cells were washed 3× with PBS and kept in complete media for imaging. Live cell images were acquired using a Zeiss CLSM 710 Spectral Confocal Laser Scanning Microscope with the 63×/1.4 Oil Plan Apo lens. Lysotracker™ and MNP colocalization was determined using the Colocalization Threshold tool in ImageJ/Fiji (NIH, Bethesda, Md.).

For transmission electron microscopy (TEM), cell monolayers were grown on Thermanox plastic substrates. The cells were treated with 0.1 mg/mL polymer-MNPs for 24 h. Post-treatment, the cells were washed with PBS and fixed in 2% paraformaldehyde/2.5% glutaraldehyde/0.15 M sodium phosphate buffer, pH 7.4, for 1 h at room temperature and stored at 4° C. until processed. Following 3 rinses with 0.15 M sodium phosphate buffer, pH 7.4, the cells were post-fixed with 1% osmium tetroxide/0.15 M sodium phosphate buffer for 1 h at room temperature. After washes in DI water, the cells were dehydrated using increasing concentrations of ethanol (30%, 50%, 75%, 100%, 100%, 10 min each) and embedded in Polybed 812 epoxy resin (Polyscienccs, Inc., Warrington, Pa.). The cells were sectioned en face to the substrate at 70 nm using a diamond knife. Ultrathin sections were collected on 200 mesh copper grids and stained with 4% aqueous uranyl acetate for 15 min, followed by Reynolds' lead citrate for 7 min (Reynolds, J. Cell Biol. 17:208-212 (1963)). Samples were viewed with a LEO EM910 transmission electron microscope (Carl Zeiss Microscopy, LLC, Peabody, Mass.) with an acceleration voltage of 80 kV. Digital images were taken using a Gatan Orius SC 1000 CCD Camera and DigitalMicrograph 3.11.0 software (Gatan, Inc., Pleasanton, Calif.).

Effect of Exposure to AC Magnetic Fields on Cell Viability:

MDA-MB-231, BT474 and MCF10A cells were seeded at 5×10³ cells/well in 2×8 MICROLON 96 well high binding plate strips (Griener Bio Inc.) and were allowed to adhere for 3 d. Cells were treated with PAA-P85-MNP complexes at various concentrations (0.05-0.5 mg/mL polymer-MNP complexes) for 24 h at 37° C., washed with acidic saline (pH 3) and exposed to AC magnetic fields of 50 kA/m or 100 kA/m and 50 Hz as specified in the legends. In the continuous mode, the cells were exposed to the field for 30 min. In the pulsed exposure mode, the cells were exposed to the field with a 10 min on, 5 min off pattern for 30 min in total. During the experiments the temperature was maintained at 37° C. All the samples were tested in triplicate. A standard MTT assay was then performed.

Intracellular Distributions of PAA-P85-MNP Complexes after Exposure to an AC Field:

MDA-MB-231, BT474 and MCF10A cells were seeded at 1×10⁵ cells/well in 8-well Lab-Tek II Chamber slides. Cells were allowed to adhere for several days and were treated with a specified dosage of Alexa Fluor®647-PAA-P85-MNPs. After 24 h, the cells were washed and then exposed to an AC magnetic field (50 Hz, 50 kA/m) using the pulsed exposure regime for a total of 30 min. Twenty four hours post exposure, the cells were treated with 100 nM of Lysotracker™ Green (λₑₓ/λₑₘ==504/511 nm) for 1 h and Hoechst 33342 nuclear stain for 15 min. Cells were washed 3× with PBS and kept in complete media for imaging. Live cell images were acquired using a Zeiss CLSM 710 Spectral Confocal Laser Scanning Microscope with the 63×/1.4 Oil Plan Apo lens.

Assessment of Lysosomal Membrane Permeabilization:

MDA-MB-231, BT474 and MCF10A cells were seeded at 1×10⁵ cells/well in 8-well Lab-Tek II Chamber slides. Cells were allowed to adhere for several days and were treated with PAA-P85-MNPs at a concentration of 0.1 mg/mL. After 24 h, the cells were washed and then exposed to an AC magnetic field (50 Hz, 50 kA/m) using the pulsed exposure regime for a total of 30 min. Three hours post exposure, cells were treated for 15 min with 10 μg/mL acridine orange stain. The cells were washed 3× with PBS and kept in complete media for imaging. Positive control cells were treated with 150 μM hydrogen peroxide for 3 h followed by thorough washing and staining with acridine orange. Live cell images were acquired using a Zeiss CLSM 710 Spectral Confocal Laser Scanning Microscope with the 63×/1.4 Oil Plan Apo lens.

Effect of Cytoskeleton Modulation on the Response to an AC Magnetic Field:

For cell viability studies, MDA-MB-231, BT474 and MCF10A cells were seeded at 5×10³ cells per well in 2×8 96-well high binding strip plates (Griener Bio Inc.) and were allowed to adhere for 2 d. The cells were treated with PAA-P85-MNPs at various doses for 24 h at 37° C. followed by washing with acidic saline. After washing to remove non-internalized polymer-MNP complexes, test cells were exposed to a 100 nM sub-lethal dosage of cytochalasin D (CD) for 1 h. After washing, the cells were exposed to the AC magnetic field and viability was tested 24 h post exposure using a MTT assay as previously described. Appropriate controls of cells exposed to just one of the compounds (either PAA-P85-MNP alone or CD alone) as well as cells without field exposure were used.

For confocal studies, MDA-MB-231, BT474 and MCF10A cells were plated on Lab-Tek II Chamber slides at a concentration of 1×10⁵ cells/well and allowed to grow overnight. The cells were then treated with 0.1 mg/mL Alexa Fluor® 647-PAA-P85-MNP for 24 h followed by thorough washing with acid saline and replacement with complete media. The cells were incubated with 100 nM CD for 1 h to enact cytoskeletal damage in a nonlethal capacity. After washing, the cells were exposed to the pulsed AC magnetic field (50 Hz, 50 kA/m) (10 min on, 5 min off, total exposure 30 min). Appropriate controls included cells not exposed to the magnetic field and untreated cells. Cells were then incubated at 37° C. for 24 h, fixed using 4% paraformaldehyde and permeabilized using 0.5% Triton-X 100. Fixed cells were stained with ActinGreen 488 (Life Technologies, Carlsbad, Calif.), a phalloidin-based actin stain and Hoechst 33342. Images were acquired using a Zeiss CLSM 710 Spectral Confocal Laser Scanning Microscope with the 63×/1.4 Oil Plan Apo lens.

Statistical Analysis:

Statistical analyses were performed using GraphPad Prism (GraphPad Software, Inc, La Jolla, Calif.). ANOVA or two-tailed Student's t-tests was used to analyze data. Where applicable, reported p-values have been adjusted for multiple comparisons using the Ryan-Einot-Gabriel-Welsch post-hoc method. Significance was reported for p<0.05.

### Synthesis of a PAA-b-P85-b-PAA Pentablock Copolymer:

Synthesis of a Br-P85-Br Macro-Initiator.

Dihydroxyfunctional P85 was reacted with 2-bromoisobutyryl bromide to make a macro-initiator that was used for polymerization of tert-butyl acrylate by atom transfer free radical polymerization. P85 (9.7 g, ˜2.1×10⁻³ mol) was dried under vacuum at 60° C. overnight, then was dissolved in anhydrous THF (100 mL) in a 250-mL round bottom flask. Triethylamine (2.3 mL, 16.5×10⁻³ mol) was added. The mixture was cooled in an ice bath and then 2-bromoisobutyryl bromide (2.0 mL, 16.5×10⁻³ mol) was added dropwise. The ice bath was removed and the mixture was stirred at room temperature for 45 h. The reaction mixture was filtered twice and THF was removed by rotary evaporation. The mixture was diluted with CH₂Cl₂ (110 mL) and then washed with a saturated aqueous NaCl solution twice. The organic layer was concentrated and precipitated in a 1:1 v:v mixture of chilled hexane and diethylether (800 mL each time) twice. The precipitate was filtered and dried under vacuum at 40° C. overnight.

Synthesis of a ptBA-b-P85-b-ptBA Copolymer.

Br-P85-Br was used as a macro-initiator for polymerization of tert-butyl acrylate. Br-P85-Br (Mₙ˜4,700 g mol⁻¹, 3.0 g, ˜6.0×10^{0.4} mol), tert-butyl acrylate (4 mL, 2.8×10⁻² mol), and dry, deoxygenated toluene (8 mL) were added into a 50-mL Schlenk flask. Oxygen was removed with three freeze-pump-thaw procedures. Cuprous bromide (0.26 g, 1.8×10⁻³ mol) and N,N,N′,N″,N″-pentamethyldiethylenetriamine (0.38 mL, 1.8×10⁻³ mol) were added quickly under nitrogen. Two additional freeze-pump-thaw procedures were applied. The Schlenk flask was sealed with parafilm and stirred at 80° C. for 19 h. After the polymerization, the reaction mixture was diluted with CH₂Cl₂ (60 mL). The catalyst was removed by filtering the reaction mixture through a neutral alumina column twice using CH₂Cl₂ as the eluent. The solution was filtered and the solvents were removed by rotary evaporation. The block copolymer was dried under vacuum at room temperature overnight.

Deprotection of ptBA-b-P85-b-ptBA to Afford PAA-b-P85-b-PAA Copolymer.

The tert-butyl ester groups were selectively removed by a previously reported procedure using trifluoroacetic acid (TFA) (Pothayee et al., J. Materials Chem. B 2:1087 (2014)). PtBA-b-P85-b-PtBA (2.4 g, ˜2.9×10⁻⁴ mol) was dried in a 100-mL round bottom flask under vacuum at 60° C. overnight. Anhydrous CH₂Cl₂ (30 mL) was added to dissolve the polymer. Trifluoroacetic acid (4.3 mL, 5.6×10⁻² mol) was added dropwise and the reaction mixture was stirred at room temperature for 24 h. The polymer was precipitated into chilled hexane (400 mL). The precipitated polymer was filtered and collected. The solid was then dissolved in THF (10 mL) and dialyzed against DI water (4 L) through a cellulose acetate membrane (MWCO 1,000 g mol⁻¹) for 48 h. The PAA-b-P85-b-PAA copolymer was recovered by freeze-drying for 2 d. The composition by weight was measured by ¹H NMR to have block molecular weights of PAA(1.9k)-PEO(1.1 k)-PPO(2.4k)-PEO(1.1k)-PAA(1.9k).

Synthesis of Polymer-MNP (PAA-b-P85-b-PAA-Magnetite Nanoparticle) Complexes:

Synthesis of polymer-MNP complexes utilized a similar procedure (Pothayee. et al., Chem. Materials 24:2056 (2012)) to that previously reported to synthesize complexes with magnetite and PEO-b-PAA. Oleic acid-coated magnetite nanoparticles (50 mg) were dispersed in anhydrous chloroform (5 mL) in a 20-mL vial. The mixture was sonicated for 10 min. Meanwhile, PAA-b-P85-b-PAA (100 mg) was charged into a separate vial equipped with a magnetic stir bar. Anhydrous N,N-dimethylformamide (DMF) (5 mL) was charged to dissolve the polymer, and the mixture was sonicated for 10 min. The magnetite dispersion was added dropwise into the polymer solution while sonicating, followed by purging with N₂ for 5 min. The reaction mixture was further sonicated for 4 h, and the water in the sonicator was changed every 30 min. The mixture was stirred at room temperature for 48 h. The mixture was precipitated into hexane (20 mL) five times. A permanent magnet was placed under the vial to attract the complex while the supernatant was decanted to remove any solvent, free oleic acid, and other residues. The remaining solid was washed with diethylether (20 mL) 3×, and the supernatant was decanted. The nanoparticles were partially dried by purging with N₂ for 2 h at room temperature, then were dispersed in de-ionized water (10 mL) and the pH was adjusted to 7.4. The dispersion was sonicated for 20 min. It was subsequently transferred to dialysis tubing with a 12-14k MWCO, and dialyzed against de-ionized water (4 L) for 24 h. Finally the polymer-MNP complexes were recovered by freeze-drying for 2 d. The polymer-MNP complexes had an intensity average diameter of 80 nm with a PDI of 0.18, as measured by DLS. The zeta potential was −59 mV.

Labeling of P85 with Atto 647:

The mono-amine P85 was prepared as reported previously (Yi et al., Bioconjug. Chem. 19:1071 (2008)). Mono-amine P85 (3.1 mg) was reacted with a 2-fold molar excess of Atto 647 N,N-hydroxysuccinimide ester (1 mg) in N,N-dimethylformamide (0.5 mL) supplemented with N,N-diisopropylethylamine (2 μL). The reaction mixture was incubated at room temperature for 5 d. The P85-Atto 647 conjugate was purified on a size exclusion column (LH-20) with methanol as the eluent. P85-Atto 647 conjugation was confirmed by thin layer chromatography (TLC) prior to use.

Fluorescence Activated Cell Sorting:

MDA-MB-231 and BT474 cells were seeded at 100K per well in 12 well plates and allowed to adhere for 3 d. After washing, they were treated with 200 μL of 0.08 μg/mL P85-Atto 647 for 1 h at 37° C. This concentration is well above the CMC of P85 (6.5×10⁻⁵ M, 0.35 mg/mL). Cells were washed with PBS 3×, harvested, and resuspended in 10% Bovine Serum Albumin for FACS analysis.

Confocal Analysis on Live Cells:

MDA-MB-231 and BT474 cells were seeded at 20K per well in Lab-Tek II Chambered Coverglass 8 well plates. Cells were allowed to adhere for 4 d, washed and treated with 200 μL of 0.08 μg/mL P85-Atto 647, Lysotracker® and Transferrin Alexa 488 for 1 h at 37° C. This concentration is well above the CMC of P85 (6.5×10⁻⁵ M, 0.35 mg/mL). Cells were washed 3× and kept in complete media for imaging. Live images were acquired using a Zeiss CLSM 510 LSM Confocal Laser Scanning Microscope with the 63×/oil immersion lens.

In Vitro Colloidal Stability of Polymer-MNP Complexes:

Polymer-MNP complexes were dispersed in DI water pH=6.5, PBS pH=7.4 or DMEM media (with 10% fetal bovine serum and 1% penicillin-streptomycin) in concentration of 1.5 mg/mL, filtered through a 0.22 m filter and incubated at 37° C. At 1, 24 and 48 h, 0.5-mL aliquots of solution were diluted with 1 mL of the corresponding media to a final particle concentration of 0.5 mg/mL and the effective hydrodynamic diameters (D_{eff}) of the polymer-MNP complexes were measured by DLS using a Zetasizer Nano ZS (Malvern Instruments Ltd., Malvern, UK). All measurements were performed in automatic mode at 25° C. All measurements were performed at least in triplicate to calculate mean values±SD.

TEM Images of MNPs in Cells:

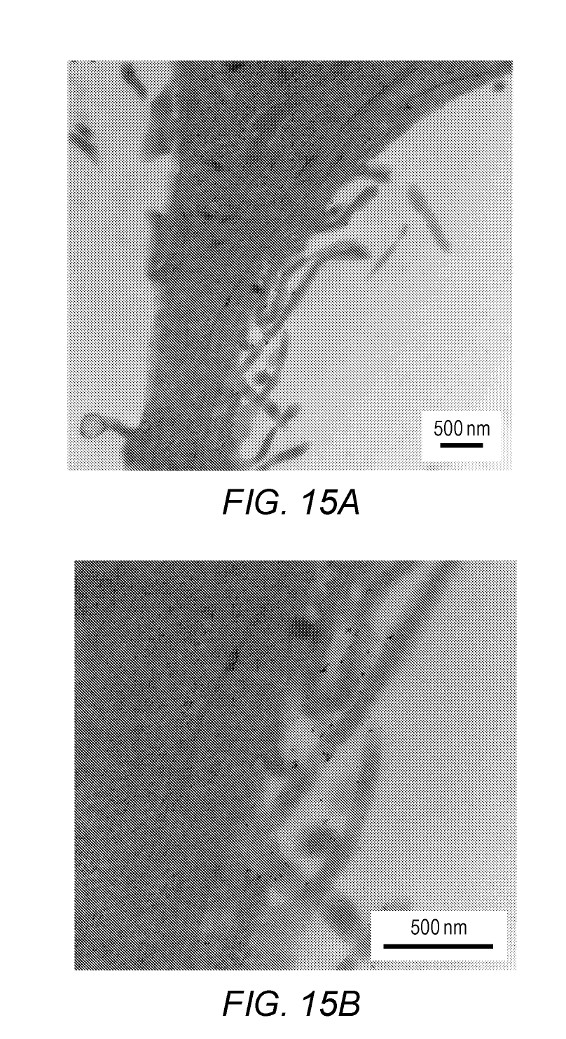

MCF7 cells were seeded in 6 well plates containing glass coverslips at a density of 1×10⁵ cells/well. Prior to treatment, cells were starved with incomplete media (no FBS) for 30 min. Cells were then incubated with MNPs for 1 h at 37° C. Cells were then preserved in 4% glutaraldehyde in formaldehyde at room temperature for 24 h, then processed for TEM analysis (FIGS. 15A-15B).

Mechanism of Cell Death by Flow Cytometry:

Cells were seeded in 8-well chamber slides and allowed to grow for several days. Cells were then treated with 0.1 mg/mL MNPs for 24 h. Following incubation, cells were washed 3× with saline and then their media was replaced. Cells were then exposed to the magnetic field. For magnetic field exposure, a 50 Hz field (50 kA/m field strength) was utilized. The pulsed regime of 10 min on, 5 min off was used. Twenty-four h post-field exposure, the Annexin V/Dead Cell Apoptosis Kit with PI from Life Technologies (Carlsbad, Calif.) was used as per the manufacturer's instructions.

### Example 2

### Quantitative Intracellular Uptake of Polymer-MNP Complexes

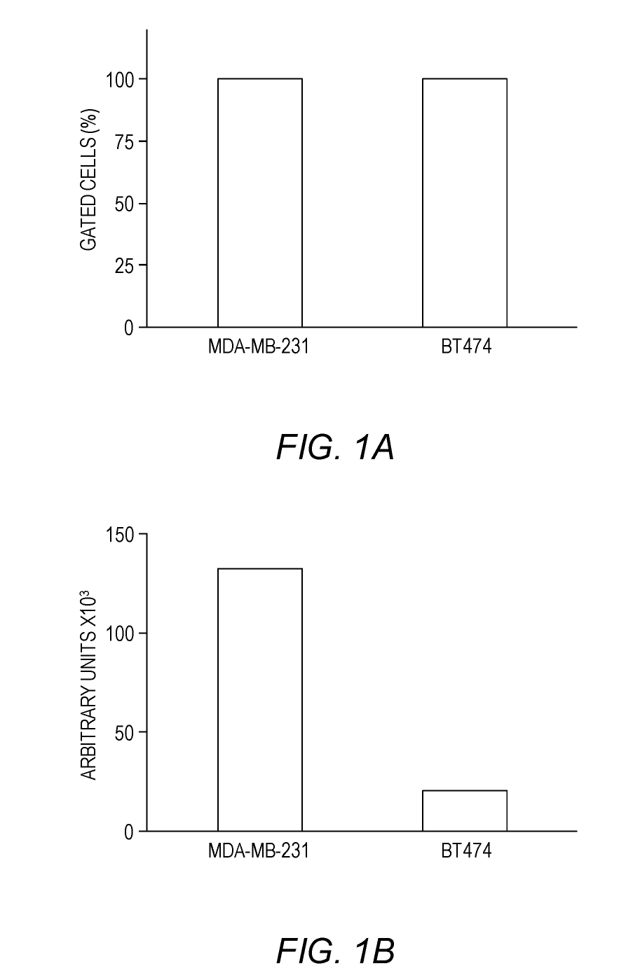

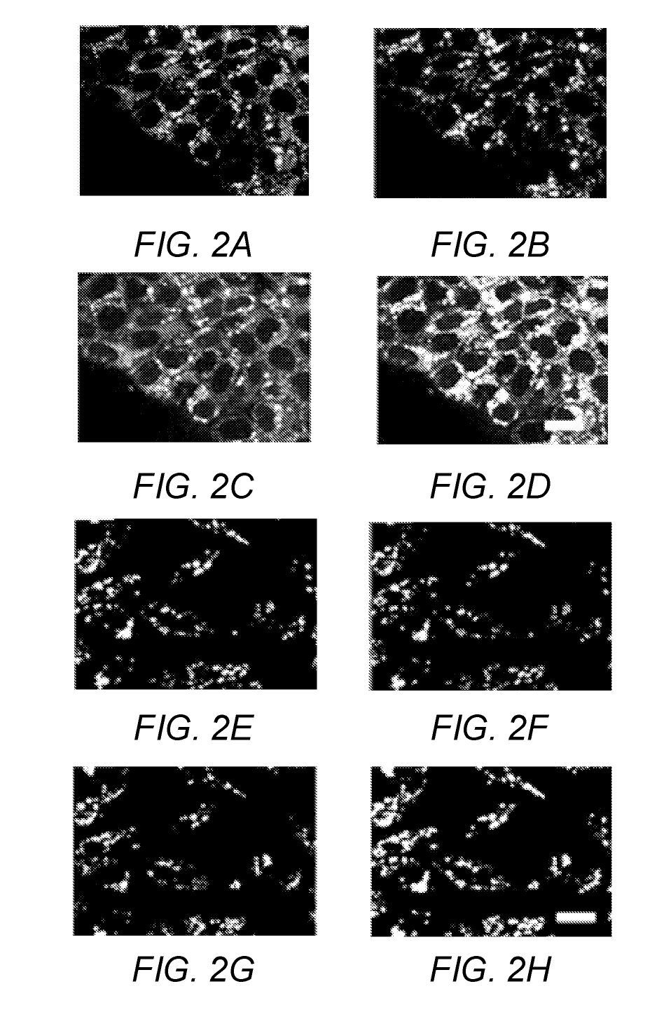

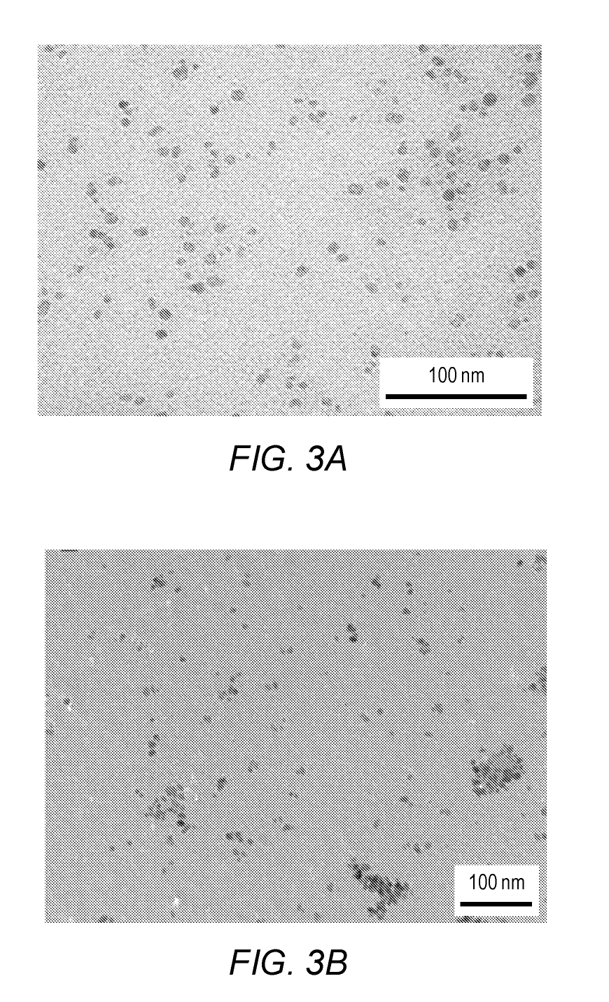

A series of block copolymers with a polyanion block and poly(ethylene glycol) (PEG) was synthesized to evaluate the effect of polymer coating composition on the polymer-MNP complexes' uptake in cancer cells. The polyanion block was either polyacrylic acid (PAA) or polymethacrylic acid (PMA), which differ in their hydrophobicity. The more hydrophobic PMA was expected to interact better with the hydrophobic cell membrane and improve particle uptake. Another strategy to improve the internalization of polymer coated MNPs was incorporation of PLURONIC® P85 (P85) into the polymer coating. P85 effectively accumulated in the cells across all the cell lines tested as was analyzed by flow cytometry (FIGS. 1A-1B) and confocal microscopy (FIGS. 2A-2H). Representative confocal microscopy images of BT474 (FIGS. 2A-2D) and MDA-MB-231 (FIGS. 2E-2H) indicate that in both cell lines P85 preferentially accumulates in lysosomes. Due to this favorable uptake pattern, P85 was incorporated in the polymer coatings of several of our tested MNP-complexes by complexation of MNPs with a PAA-b-P85-b-PAA pentablock copolymer. The physicochemical characteristics of the formed polymer-MNP complexes are summarized in Table 1. The sizes (D_{eff}) of the polymer-MNP complexes were in the range of 30-70 nm with ζ-potential values of −35 to −50 mV. The polymer content in all the complexes was around 60 wt % as measured by thermogravimetric analysis (TGA), and this was in excellent agreement with the iron concentration measured by ICP-MS. All the polymer-MNP complexes were small clusters with several MNP cores incorporated together as observed by TEM (FIGS. 3A-3B).

TABLE 1
Summary of polymer-MNP complexes used in this study

| | Polymer | | | | | Polymer |
|---|---|---|---|---|---|---|
| | block | | | | ζ- | content in |
| Polymer | lengths | | D_{eff} | | potential, | complex |
| composition | (kDa)^{a} | Abbreviation | (nm)^{b} | PDI^{c} | mV^{d} | (%, w/w)^{e} |
| Polyacrylic | 7.7K-2K | PAA-PEG- | 67.0 ± 3.9 | 0.19 ± 0.01 | −39.01 ± 1.17 | 59.5 |
| acid-PEG | | MNP | | | | |
| Polymethacrylic | 7.2K-2K | PMA-PEG- | 55.7 ± 0.7 | 0.18 ± 0.01 | −47.03 ± 0.95 | 63.1 |
| acid- | | MNP | | | | |
| PEG | | | | | | |
| 1:1 w/w | 7.7K-2K/ | PAA-PEG/ | 38.2 ± 0.1 | 0.29 ± 0.01 | −44.23 ± 2.61 | 64.1 |
| blend of | 4.6K-3K | PAA-P85- | | | | |
| Polyacrylic | | MNP | | | | |
| acid-PEG | | | | | | |
| and | | | | | | |
| Polyacrylic | | | | | | |
| acid-P85 | | | | | | |
| Polyacrylic | 1.9K-4.6K- | PAA-P85- | 30.2 ± 0.1 | 0.41 ± 0.001 | −34.31 ± 5.2 | 65.3 |
| acid-P85- | 1.9K | MNP | | | | |
| Polyacrylic | | | | | | |
| acid | | | | | | |

^{a}Polymer block length is defined as the length of the polyacid block-length of the PEG or P85 block.
^{b,c,d}D_{eff}, PDI and ζ-potential were measured by DLS with Nano-ZS in de-ionized water at concentration of 0.5 mg/mL at 25° C. D_{eff} is reported as an intensity averaged diameter.
^{c}Polydispersity index.
^{e}Polymer content in the complex was measured by thermogravimetric analysis (TGA).
Briefly, 10-15 mg samples were heated at 10° C./min to 110° C., held isothermally for 15 min and then heated at 10° C./min to 700° C.

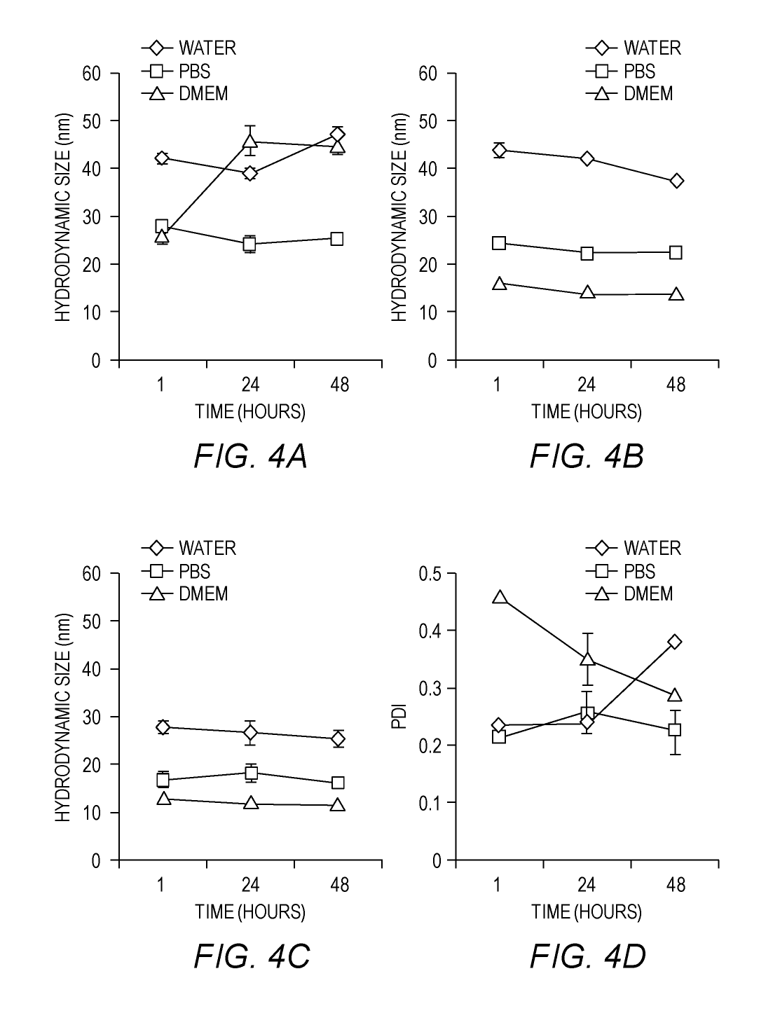

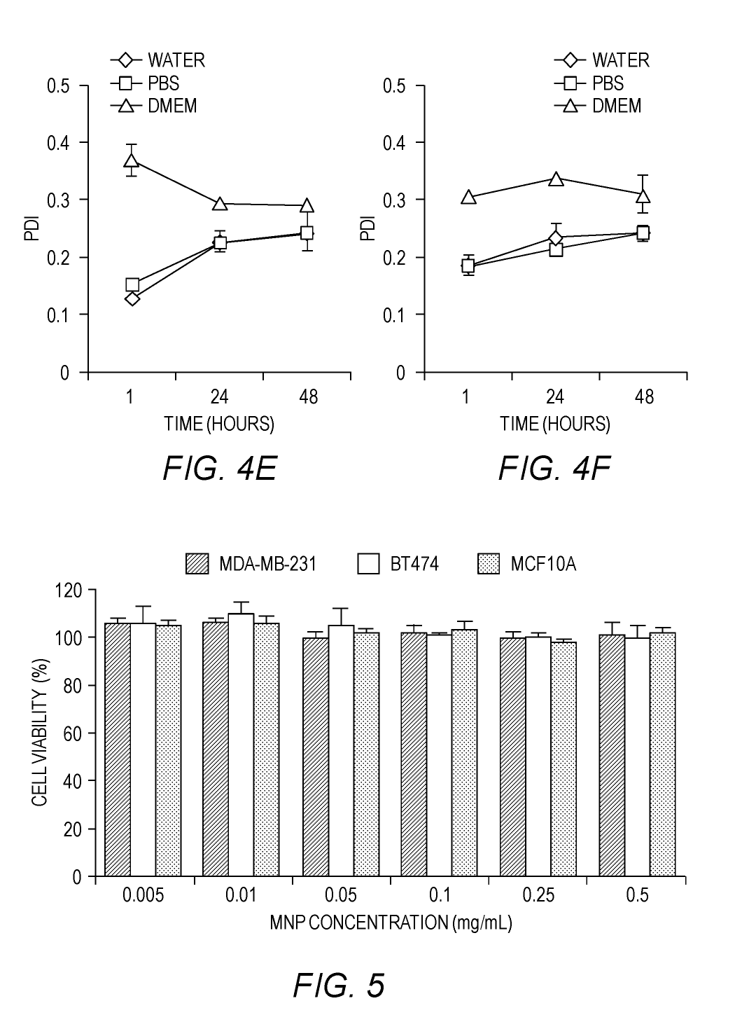

All the polymer-MNP complexes were stable in aqueous dispersion for over 48 hours under different ionic environments (DI water, PBS, and complete media) (FIGS. 4A-4F). The saturation magnetization values of all the clusters were in the 60-70 emu/g Fe₃O₄ range. Preliminary cytotoxicity studies showed that all tested polymer-MNP complexes were minimally toxic in MDA-MB-231, BT474 and MCF10A cells at all tested concentrations (FIG. 5).

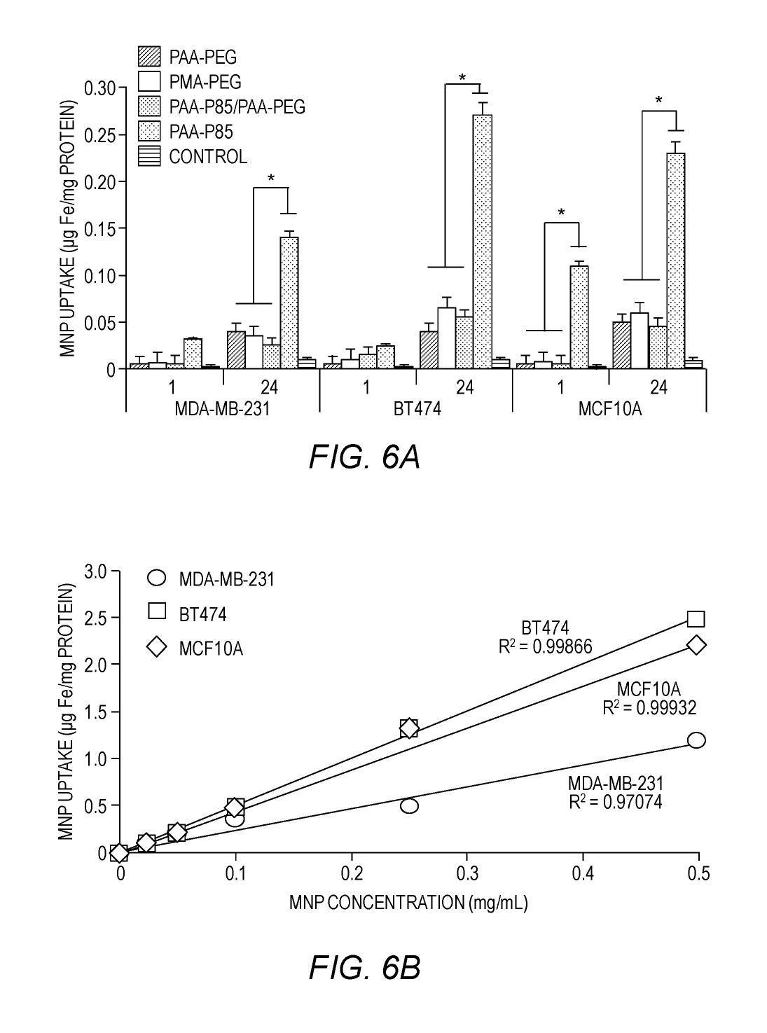

Internalization of the polymer-MNP complexes was evaluated following 1 h and 24 h of incubation and was determined by the amount of Fe/mg protein in the cells (FIG. 6A). All polymer-MNP complexes showed time and concentration dependent uptake in all experimental cell lines. PMA-PEG-MNP showed slightly enhanced uptake compared to PAA-PEG-MNP, especially in BT474 cells but these differences were not statistically significant. Incorporation of P85, with its relatively hydrophobic central block, into the polymer chain effectively promoted internalization of the PAA-P85-MNPs. Interestingly, this effect of PAA-P85 was lost when PAA-P85 was mixed with PAA-PEG in the PAA-PEG/PAA-P85 blend coated MNP. Comparable accumulation of PAA-P85-MNP was observed in BT474 and MCF10A cells after 24 h while uptake in MDA-MB-231 was lower (FIG. 6B). Due to significantly higher uptake the following studies focused exclusively on the PAA-P85-MNP complexes.

### Example 3

### Intracellular Distribution of PAA-P85-MNP

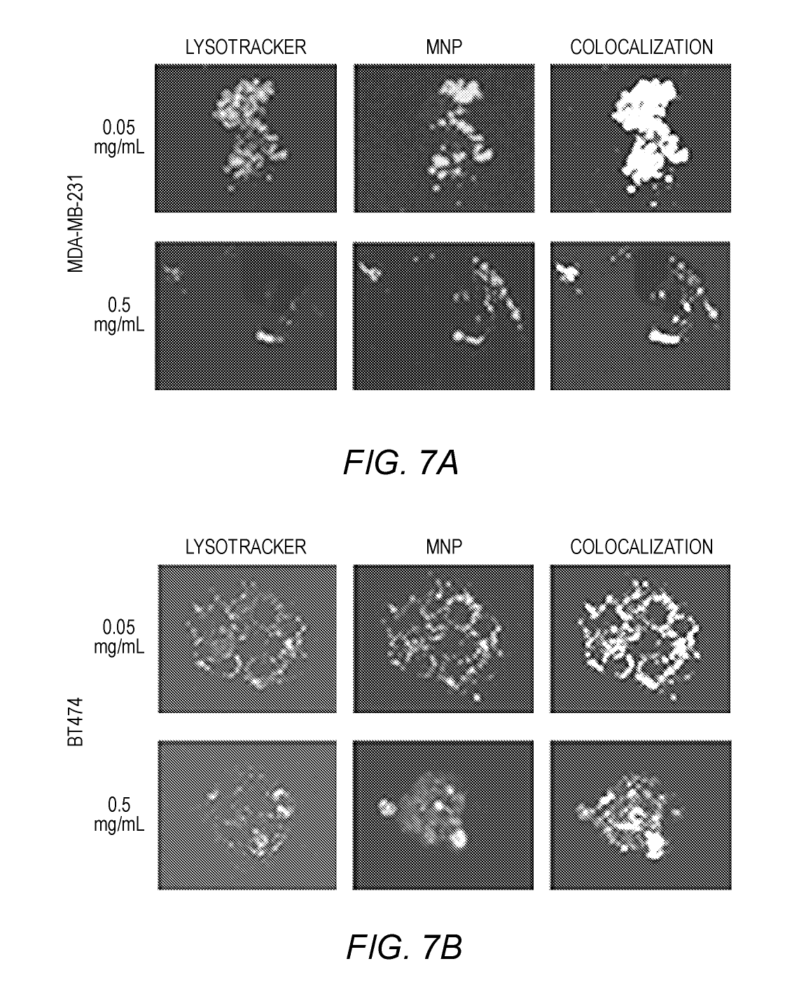

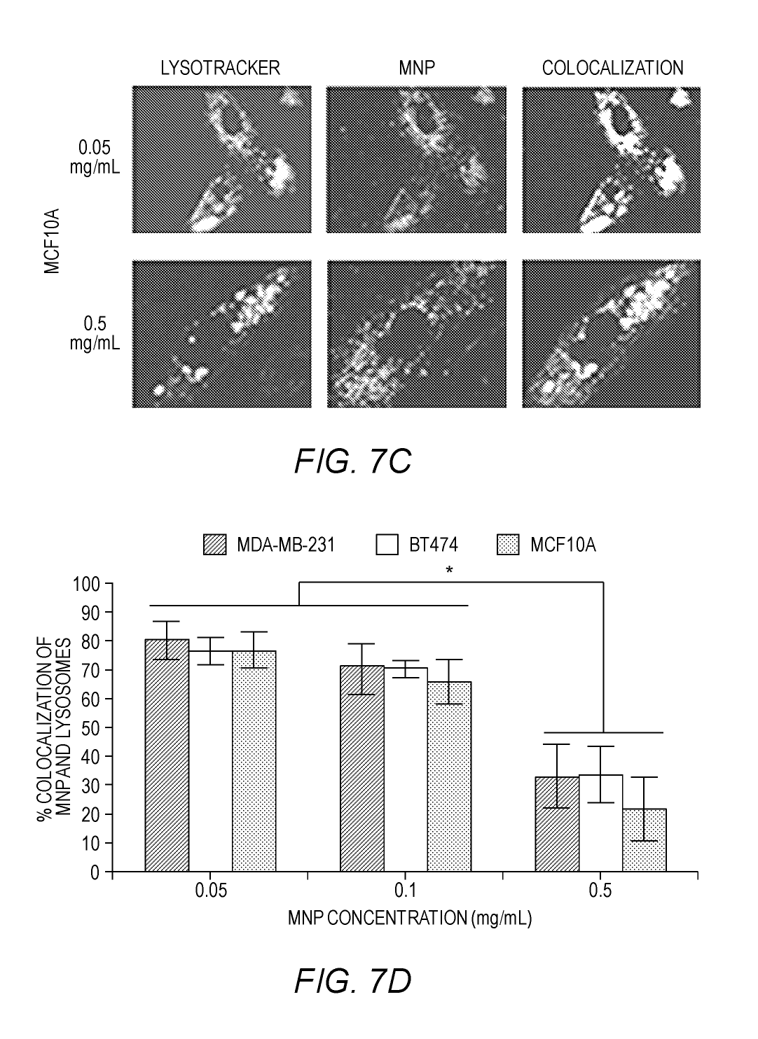

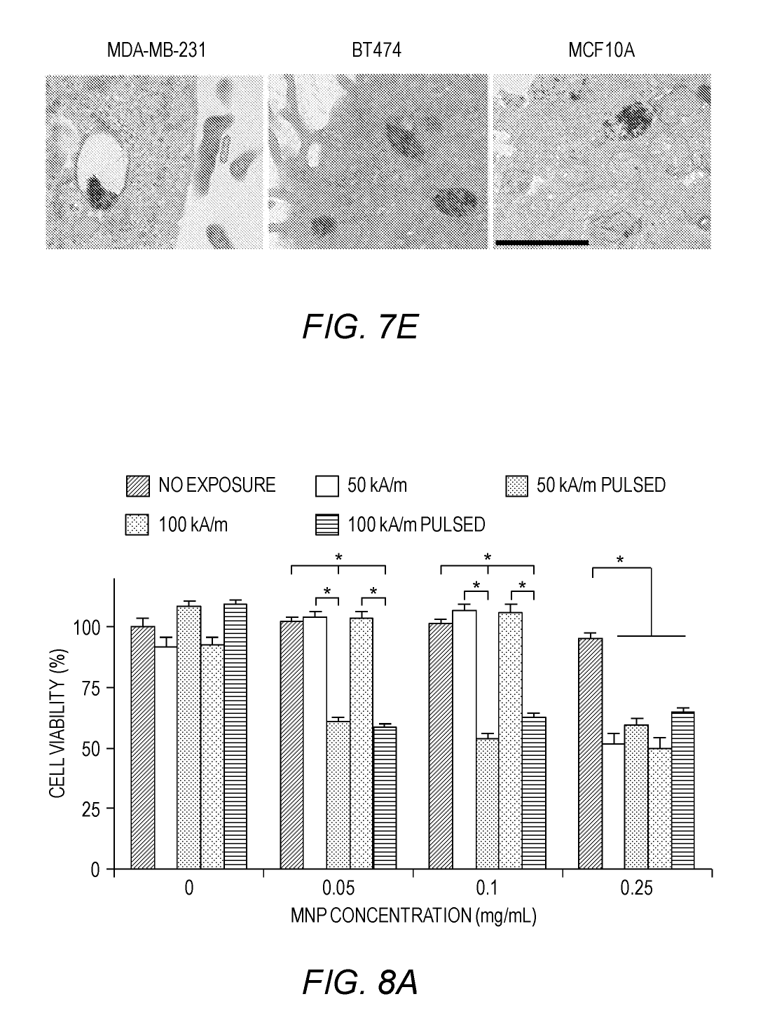

Intracellular distributions of the PAA-P85-MNP complexes were studied by confocal microscopy in MDA-MB-231, BT474 and MCF10A cells. For this experiment, the nuclei were labeled with DAPI (blue), lysosomes were labeled with Lysotracker Green and the PAA-P85-MNPs were labeled with Alexa Fluor®647 (red). The overlap of the Lysotracker and MNP labels indicates colocalization. Our preliminary studies suggested that the intracellular localization of the PAA-P85-MNPs varied at different dosing concentrations. Therefore, this study dosed with both a low (0.05 mg/mL or 0.1 mg/mL) and high (0.5 mg/mL) concentration. FIGS. 7A-7C show representative confocal images of intracellular distributions of Alexa Fluor®647-PAA-P85-MNP complexes following incubation for 24 h. As can be seen at the low concentration of 0.05 mg/ml PAA-P85-MNP complexes are accumulated in lysosomes, while at the high concentration of 0.5 mg/ml the PAA-P85-MNPs also spread throughout the cytoplasm. These observations are further confirmed by the colocalization quantitative data shown in FIG. 7D. This data shows that in all three cell lines colocalization of the Polymer-MNP complexes with lysosomes remains quite high (80%) at low exposure concentrations of 0.05 and 0.1 mg/mL, but drops off significantly to about 30% at the high exposure concentration of 0.5 mg/mL. FIG. 7E shows TEM data of PAA-P85-MNPs in cells to further confirm high amounts of lysosomal accumulation.

### Example 4

### In Vitro Exposure to Super Low Frequency AC Field

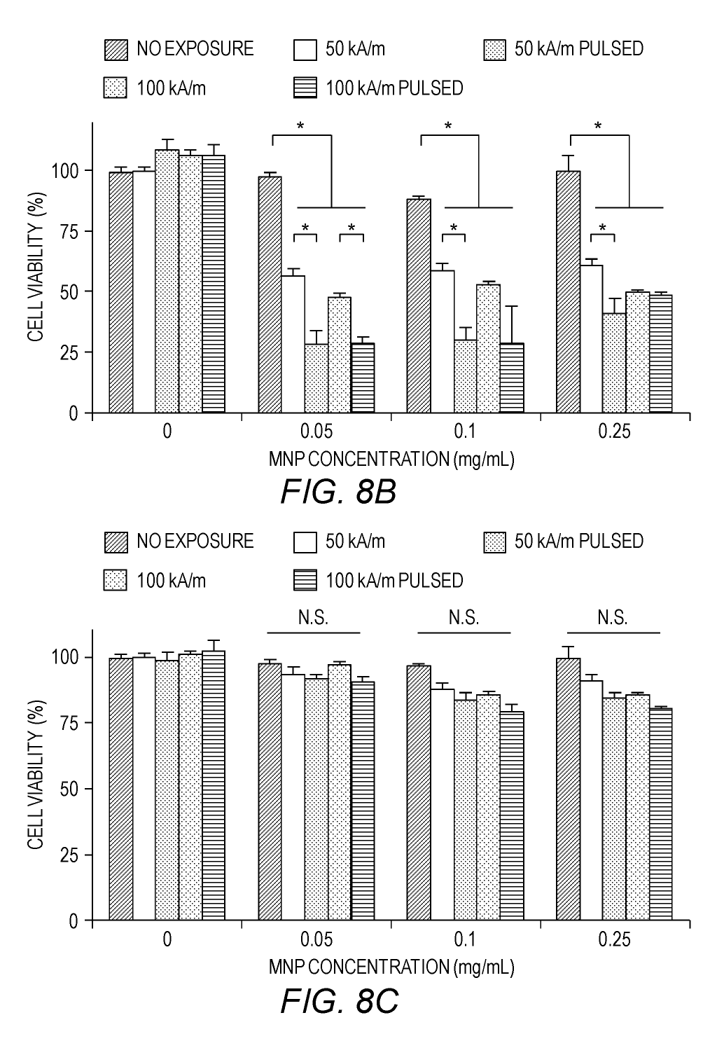

Following incubation with various concentrations of PAA-P85-MNPs for 24 h, the cells were exposed to a super low frequency AC magnetic field (50 Hz) with field strengths of 50 or 100 kA/m utilizing two exposure regimes termed ‘continuous’ (30 min) or ‘pulsed’ (10 min on, 5 min off, total 30 min on). A remarkable difference in the response of cancerous (MDA-MB-231 and BT474) versus non-cancerous (MCF10A) cells was observed. There was a significant reduction in cell viability at as low as 0.05 mg/mL of PAA-P85-MNPs in both MDA-MB-231 (FIG. 8A) and BT474 cells (FIG. 8B) regardless of the field exposure regime utilized. However, as seen in FIG. 8C, despite similar internalization rates and MNP concentration inside the MCF10A cells there was no noticeable decrease in cell viability after AC magnetic field exposure (all tested regimes). For the cancerous MDA-MB-231 and BT474 cells, the effect on cell viability did not occur in a dose dependent manner and was not enhanced with increased field strength. Interestingly, in the MDA-MB-231 cell line, field exposure using the continuous field regime caused little toxicity up to 0.25 mg/mL PAA-P85-MNP complexes while in the BT474 cells, this same exposure regime caused a 50% decrease in cell viability following incubation with only 0.05 mg/mL PAA-P85-MNP complexes. However, in both cell lines, the pulsed field regime was significantly more effective compared to the continuous field regime (50% for pulsed versus 100% cell viability for continuous field in MDA-MB-231 and 25% for pulsed versus 50% cell viability for continuous field in BT474). Exposure of the cells in the absence of PAA-P85-MNPs to either a continuous or pulsed field regime remained minimally toxic for both cell lines. Cell viability after exposure to 0.5 mg/mL MNPs was assessed but did not yield any higher efficacy in any of the cell lines. Due to these results, further experiments were done using a 50 kA/m field strength and the pulsed field regime.

These results show that the BT474 cells are more sensitive to the treatment than the MDA-MB-231 cells, and the healthy MCF10A cells do not seem to be affected at all. To further determine a mechanistic understanding of this observation, we first needed to determine if lysosomal membrane permeabilization (LMP) or cellular heating was responsible for the observed cell death. It has been determined, based upon our previous experimental results as well as theoretical calculations, that the observed effects cannot be explained by bulk or surface heat (Klyachko et al., Angew Chem. Int. Ed. Engl. 51:12016 (2012)). Previously we have clearly shown that exposure of PAA-P85-MNP dispersions to super low frequency AC magnetic fields does not result in a temperature increase of the surrounding medium, and that changes in the physical structure of a conjugated enzyme were significantly different from a temperature-induced structural deformation (Klyachko et al., Angew Chem. Int. Ed. Engl. 51:12016 (2012)). Thus, we can conclude that the cell death observations are not due to heating effects.

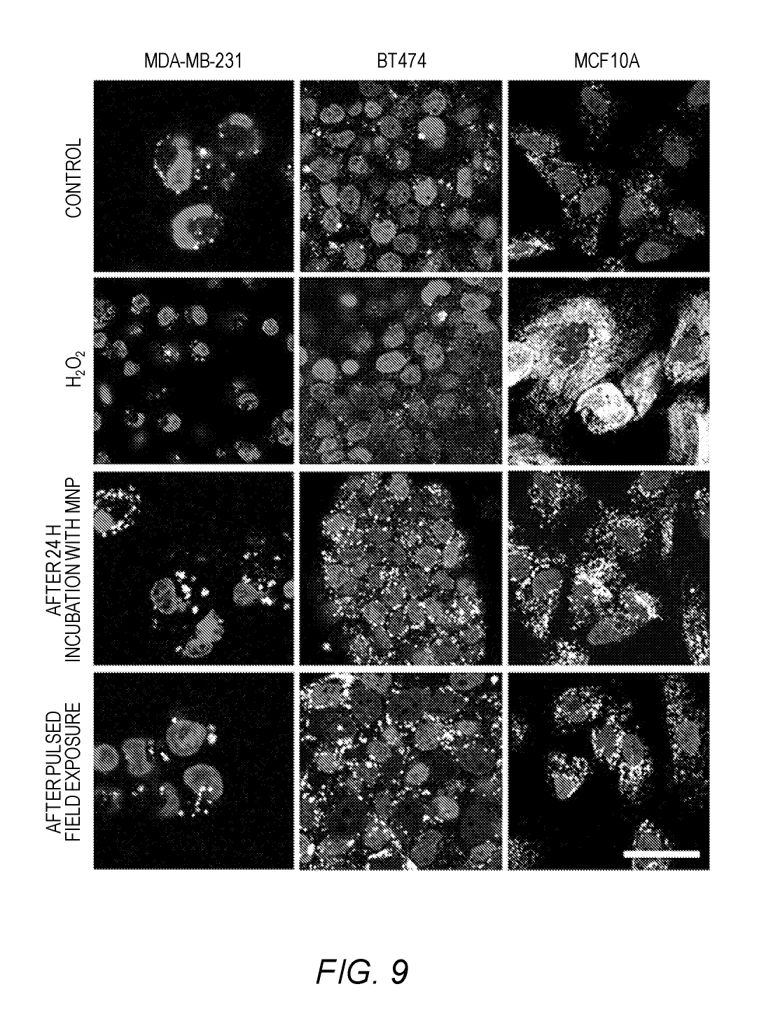

Previous studies have indicated that exposure of cells to alternating current magnetic fields can result in mechanical disruption of the lysosomes resulting in LMP and subsequent death through these lysosomal pathways (Zhang et al., ACS Nano 8:3192 (2014); Sanchez et al., ACS Nano 8:1350 (2014)). To evaluate if a similar phenomenon was occurring in our system, cells were incubated with Alexa Fluor 647-PAA-P85-MNP and Lysotracker Green and exposed to the 50 kA/m, 50 Hz field using the pulsed field regime. Disruption of lysosomes would result in leakage of the acidic content as well as of Lysotracker Green resulting in loss of punctuate fluorescence. Confocal images showed no evidence of lysosomal disruption (FIG. 9) in any of the cell lines. Lysotracker Green remained colocalized with Alexa Fluor®647-PAA-P85-MNPs without a noticeable decrease in Lysotracker Green fluorescence in all cell lines.

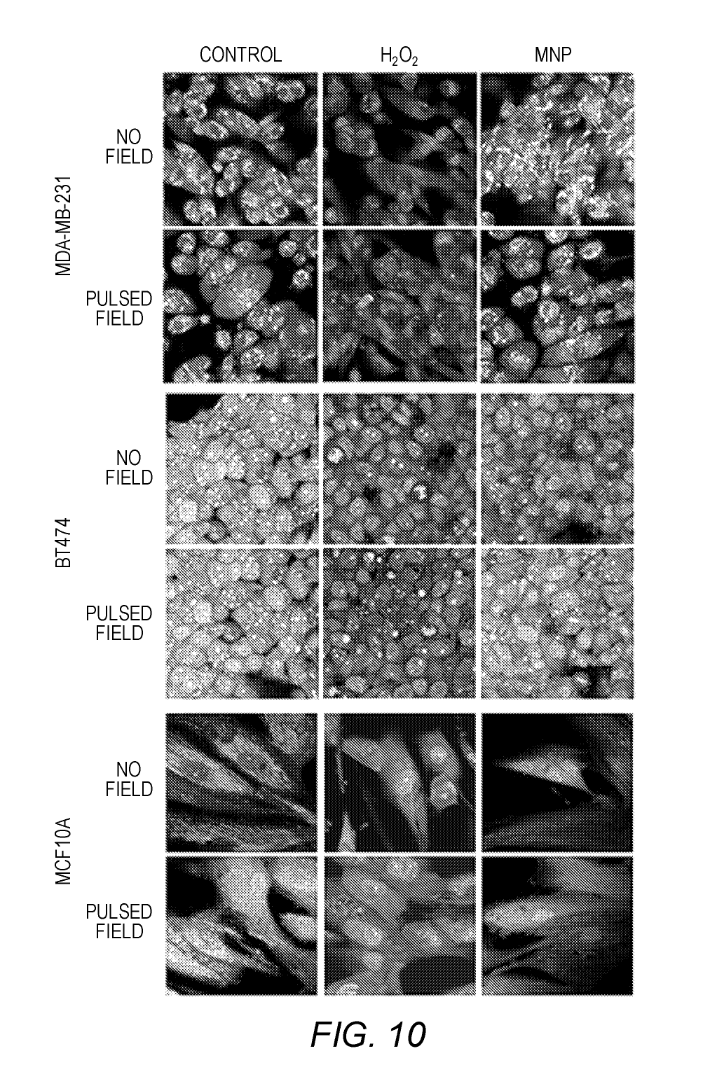

An acridine orange assay, a more robust method to detect LMP, was also conducted. Acridine orange is a lysosomotropic stain that can be used to measure the lysosome membrane functionality. The stain is excited by UV light and emits red/orange fluorescence when in lysosomes and green fluorescence when present in the nucleus or cytosol. Cells with intact lysosomes display punctuate red/orange fluorescence but this red/orange fluorescence reduces significantly after LMP (Trincheri et al., Carcinogenesis 28:922 (2007); Michallet et al., J Immunol. 172:5405 (2004); Boya et al., J. Exp. Med. 197:1323 (2003)). Hydrogen peroxide was used as a positive control because it is known to induce LMP (Antunes et al., Biochem. J. 356:549 (2001)). FIG. 10 shows that MNP incubation along with pulsed field exposure does not cause loss of lysosomal fluorescence as observed in the positive hydrogen peroxide control. The lysosomes retain the punctuate red/orange fluorescence before and after field exposure in all three cell lines, which indicates a lack of LMP.

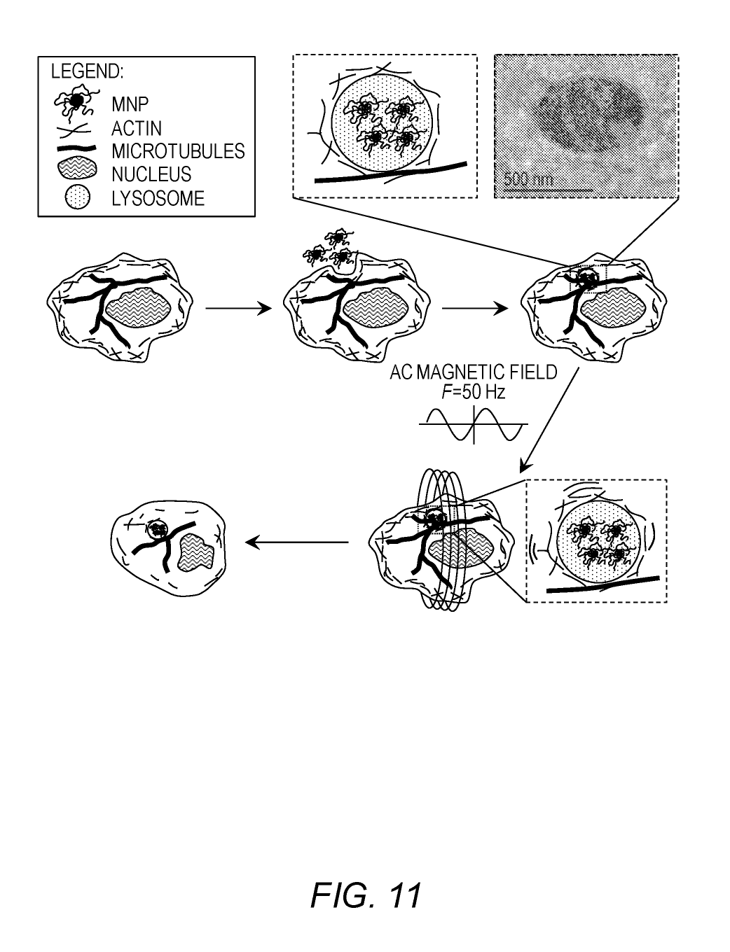

Once heating and LMP were eliminated as potential explanations for our observations, we looked to the differing cytoskeletal architectures of the cell lines for a mechanism. Cytoskeletal damage as a cause of cell death has been well reported in the literature. Actin filaments are one of the main components involved in maintaining cell structure as well as assisting with transport of organelles and vesicles throughout the cell. Previous research has shown that interference with cytoskeletal components can cause cessation of the cell cycle and lead to apoptosis (Atencia et al., Vitam. Horm. 58:267 (2000); Ndozangue-Touriguine et al., Biochem. Pharmacol. 76:11 (2008)). Lysosomes are anchored to microtubule highways and highly associated with actin filaments. The hypothesis for this system is that the PAA-P85-MNPs accumulate in lysosomes and upon remote actuation by the AC magnetic field can rotate inside of the lysosome, thus inducing torques and shear stresses on the underlying cytoskeleton, all without causing lysosomal leakage. A schematic of this event progression can be seen in FIG. 11. The cytoskeleton in cancerous cells is more sensitive to mechano-transduction leading to subsequent damage and cell death. Thus, it is suggested that while the generated forces are insufficient to cause damage to the underlying cytoskeleton of the stiffer, benign cells, less mechanical force is required to cause cytoskeletal deformation to the cytoskeleton of non-cancerous cells (Swaminathan et al., Cancer Res. 71:5075 (2011); Wakatsuki et al., J. Cell Sci. 114:1025 (2001); Lee et al., Biophys. J. 102:2731 (2012)).

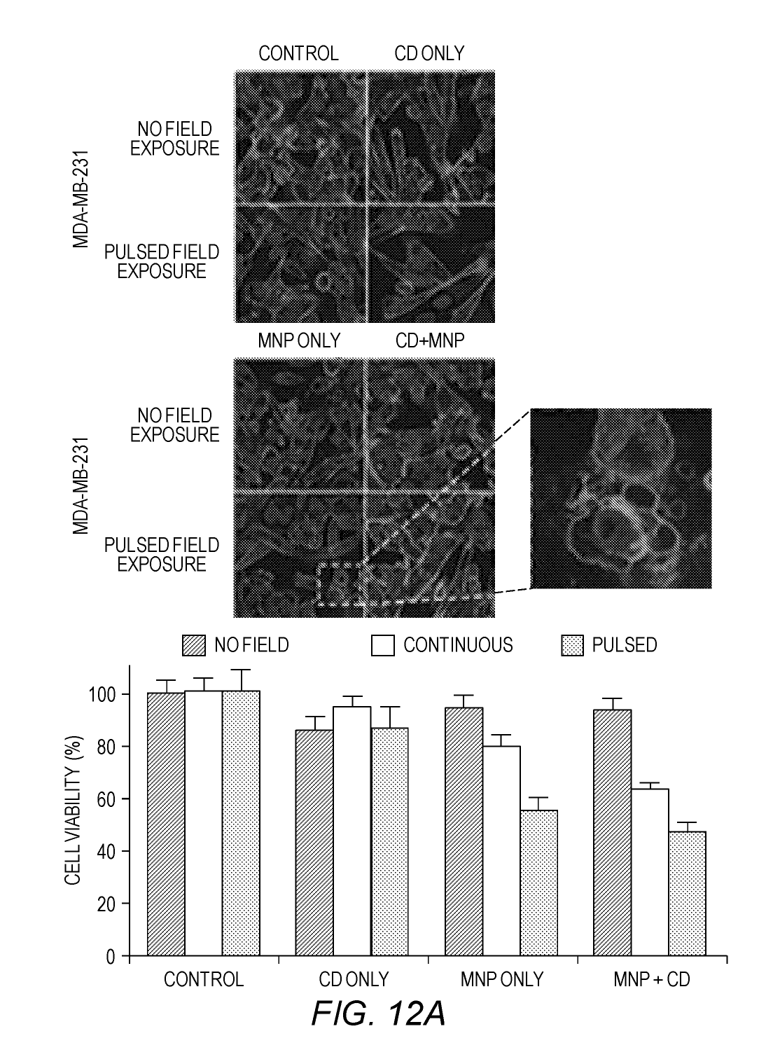

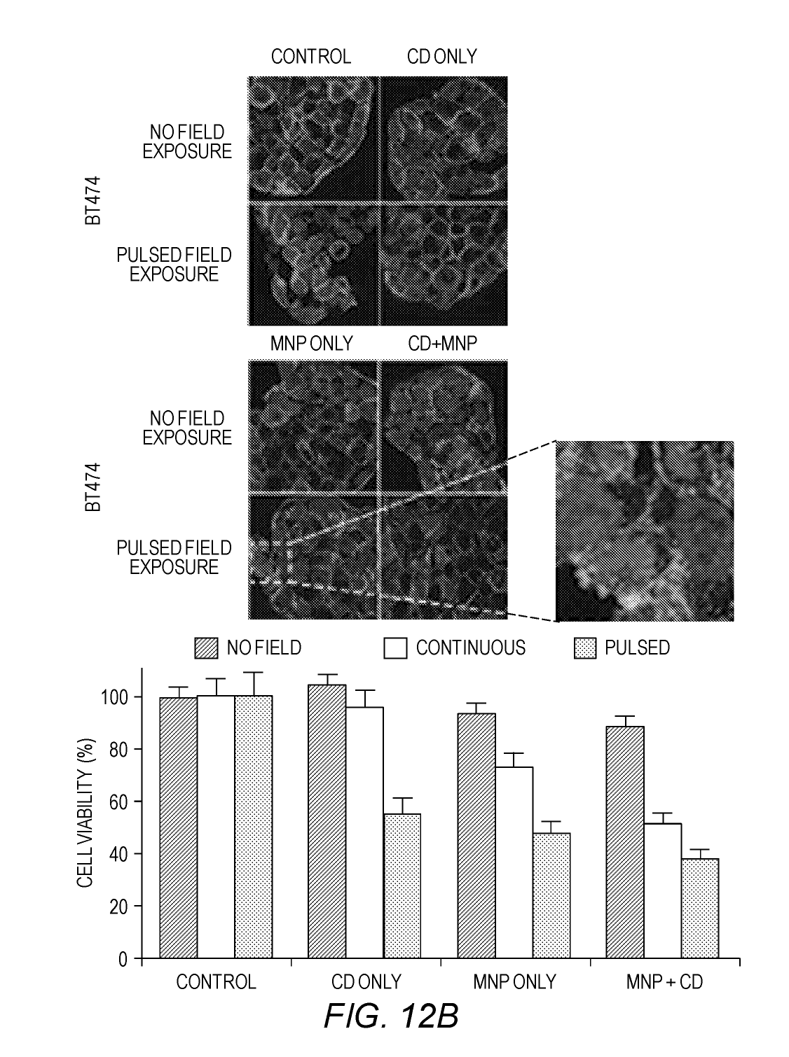

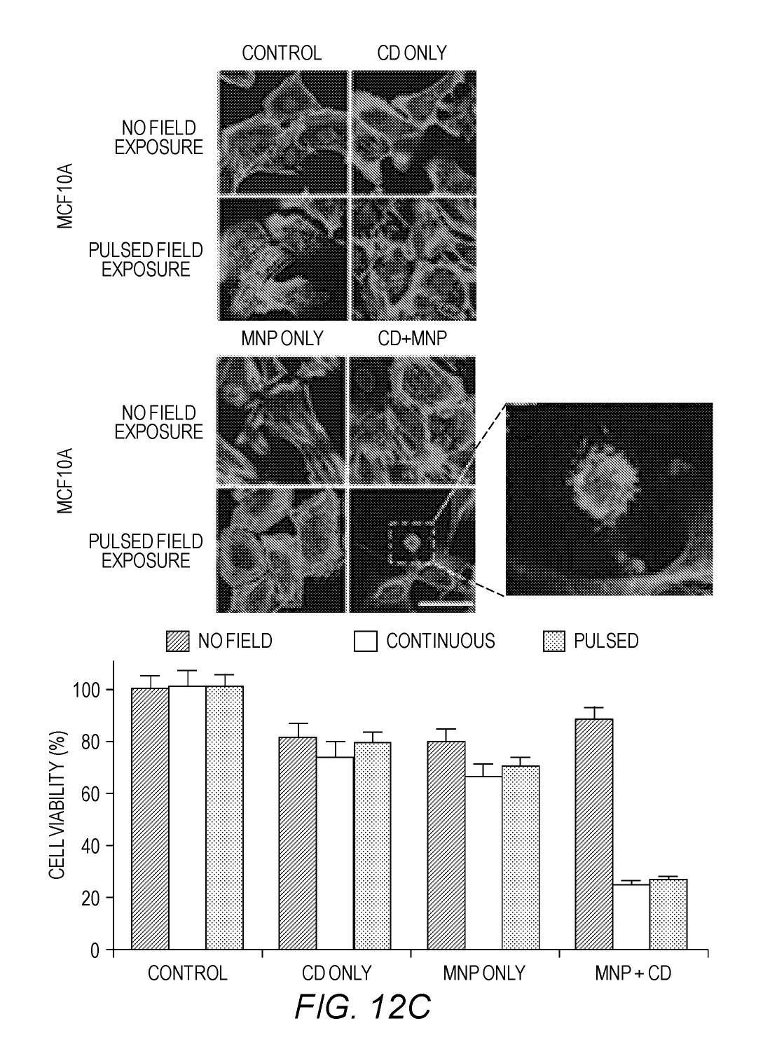

The theory of actin damage as the cause of cell death was studied by first determining the effect of the AC magnetic field on actin structure using confocal microscopy (FIGS. 12A-12C). MDA-MB-231 and BT474 control cells show an actin filament structure very typical of cancer cells while the nontumorigenic MCF10A cells show actin structures very typical of healthy epithelial cells. Following exposure to 0.1 mg/mL of Alexa Fluor 647® labeled PAA-P85-MNPs and a pulsed 50 Hz, 50 kA/m AC magnetic field, the confocal images revealed significant disruption of the actin cytoskeleton in the cancerous MDA-MB-231 and BT474 cells but not in the nontumorigenic MCF10A cells (FIGS. 12A-12C). This is in excellent agreement with the previously discussed cytotoxicity data (FIGS. 8A-8C). To further test the correlation between the mechanical properties of the cells and treatment effects, the cells were incubated with Cytochalasin D (CD). CD disrupts actin polymerization and in sub-lethal doses decreases the mechanical stiffness of cells (as measured by Atomic Force Microscopy) (Wakatsuki et al., J. Cell Sci. 114:1025 (2001)). Therefore, exposure of non-cancerous cells to CD reduces their stiffness to the levels comparable to cancer cells (Wakatsuki et al., J. Cell Sci. 114:1025 (2001)). Notably after exposure to CD and MNPs the pulsed AC magnetic field regime enacts significant cytoskeletal damage in MCF10A cells as can be seen in the insert of FIG. 12C. The damage is comparable to the damage observed in the cancerous cells following exposure to the MNPs and pulsed AC magnetic field (FIGS. 12B-12C). No significant differences in the cytoskeleton structure were observed in cancerous cells incubated with CD alone following exposure to a pulsed AC magnetic field.

Cell viability data confirmed these observations. Addition of CD to MNP exposed MCF10A cells sensitizes them to both the continuous and pulsed AC magnetic field regimes (FIGS. 12A-12C). The MCF10A cell viability decreased to 25% following exposure to the 50 Hz, 50 kA/m AC field. It was also interesting to see that CD appeared to sensitize the cancer cells to forces created by the MNP in the continuous field regime.

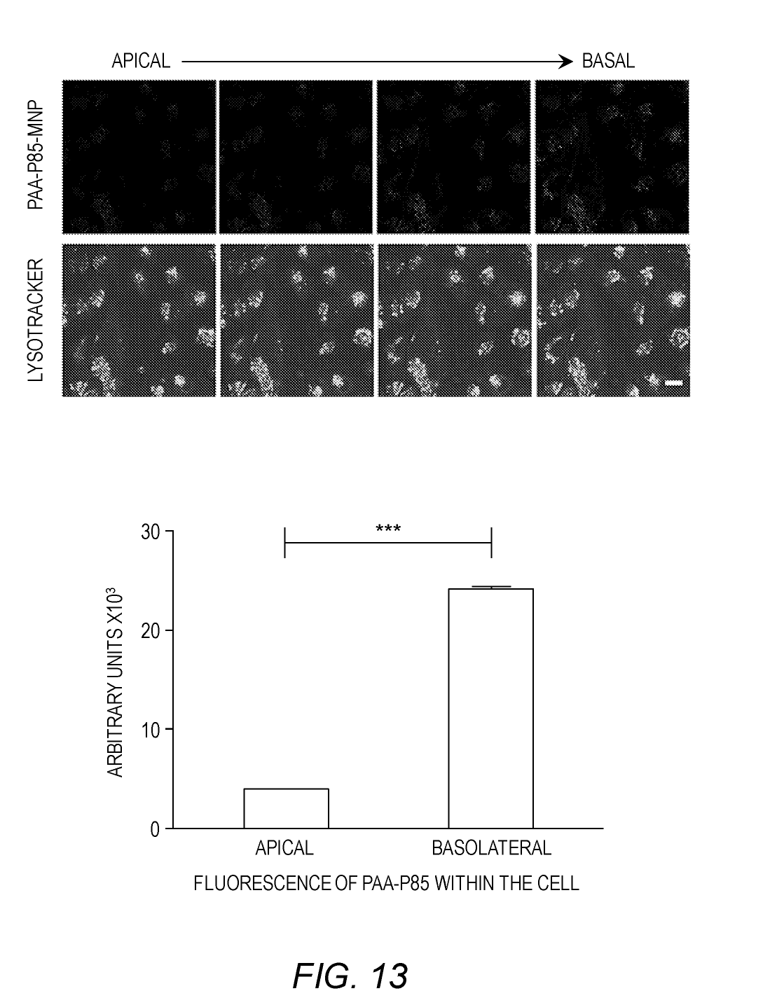

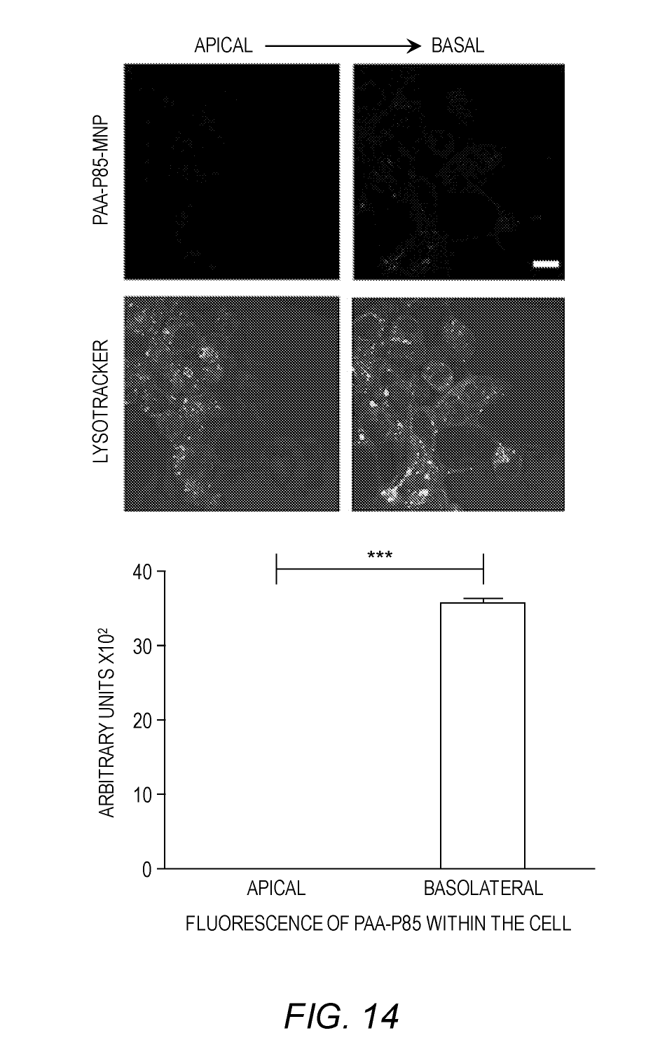

The proposed mechanism of mechanical disruption of the cytoskeleton is in very good agreement with the differences in cytotoxicity observed for MDA-MB-231 cells versus BT474 cells. BT474 cells grow in multilayer colonies and their complex cytoskeletal structure is very important to their growth. Interestingly, we have observed colocalization of PAA-P85-MNPs with the basal cells rather than in the top layer (FIGS. 13 and 14). It may be that when the cytoskeletons of the basal cells in the colony are compromised, this causes a subsequent loss to the apical cells in the colony as well which results in the lower cell viability we observed in FIGS. 8A-8C.

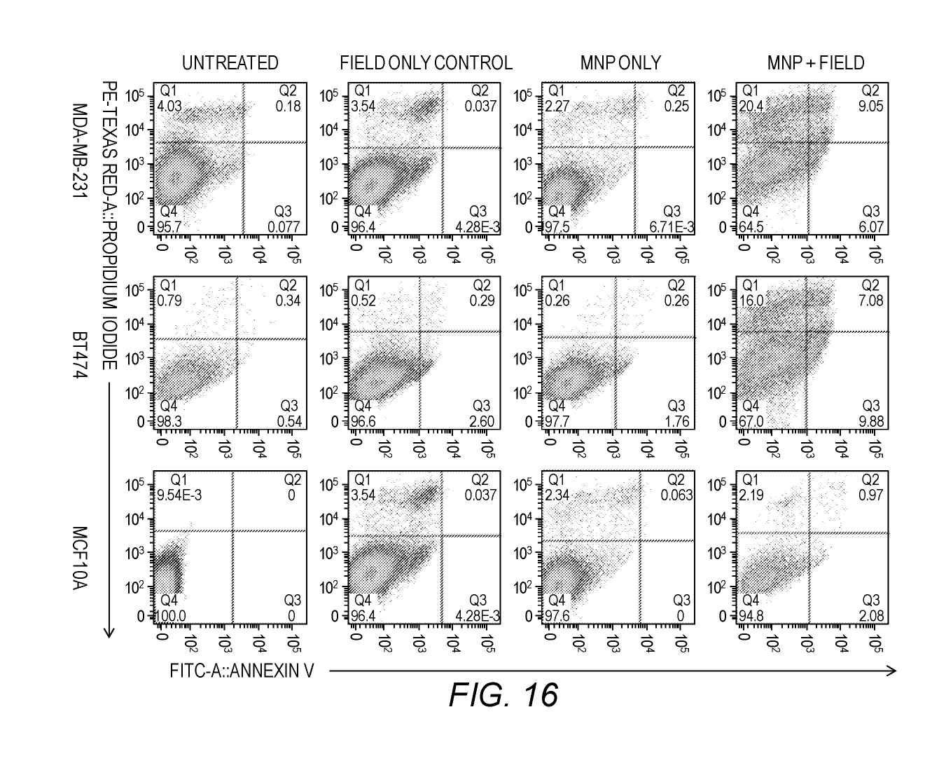

The results seen in FIG. 16 corroborate data found through MTT assays. In this figure, Q1 indicates purely necrotic cells, Q2 is a mixture of late stage apoptotic cells and necrotic cells, Q3 is early stage apoptotic cells and Q4 is live cells. This further confirms that the MCF10A cells remain unaffected by the combination of MNP and pulsed field exposure. Similarly, the MDA-MB-231 and BT474 cells yielded significant cell death after MNP and field exposure. The figure shows that the majority of cells are in late stage apoptosis or necrosis but it is important to note that this is a snapshot of the cell death after 24 hours. Therefore, it is possible that cells that underwent apoptosis soon after field exposure may become sensitive to the PI dye by the 24-hour timepoint.

We observed a new mechanism of toxicity of MNPs in non-heating super low frequency AC magnetic fields to cancerous cells that involves cytoskeletal disruption, and it can be selectively enacted upon cancerous cells while leaving healthy cells intact. The selective cytotoxic effect was dependent on the cell mechanical properties rather than on intracellular uptake disparities between cancerous and healthy cells reported elsewhere (Wen et al., Int. J. Nanomedicine 9:2043 (2014)). Notably, cancerous and non-cancerous cell lines differ in mechanical properties of the cytoskeleton. Cancerous cells are mechanically softer than their benign counterparts due to their need to remodel during transformation and metastasis (Swaminathan et al., Cancer Res. 71:5075 (2011)). For example, the Young's modulus of malignant MDA-MB-231 cells is less than half that of the non-malignant MCF10A cells (Nikkhah et al., J Biomech. 44:762 (2011)). It has previously been shown that MNPs conjugated to signaling proteins can control the assembly of cytoskeletal components such as microtubules in an applied magnetic field (Hoffmann et al., Nat. Nanotechnol. 8:199 (2013); Hoffmann et al., ACS Nano 7:9647 (2013)). It was also shown that MNPs under AC magnetic fields can form linear aggregates (Saville et al., J Colloid Interface Sci. 424:141 (2014)). In addition, in high frequency magnetic fields, MNPs can oscillate mechanically and generate ultrasound waves (Carrey et al., Appl. Physics Lett. 102:232404 (2013)). While the movement of individual particles cannot induce forces high enough to generate biological responses, forces generated by an assembly of MNPs, such as those observed here in lysosomes, are sufficient to induce cellular responses (Carrey et al., Appl. Physics Lett. 102:232404 (2013)). We have previously reported that exposure to an AC field can cause mechanical movement of MNPs, which generates stress forces and deformation of the surrounding polymer coating and any attached biological molecules. In that study, PAA-PEG coated MNPs with an average MNP core diameter of 8 nm and enzymes conjugated to the particles' surface were reported (Klyachko et al., Angew Chem. Int. Ed. Engl. 51:12016 (2012)). The AC fields used herein (50 Hz, 50 to 100 kA/m) can produce forces ranging from several dozen to ˜300 pN, which can increase if single particles form small aggregates with a greater net magnetic moment (Golovin et al., J. Control. Release 219:43 (2015)). Such forces may exceed the strength of the filaments in the cells and result in their damage (Suresh, Acta Biomater. 3:413 (2007)). The literature states that actin-actin bonds will break at 600 pN under straight pulling and at 320 pN under twisting forces (Noy, A. Handbook of molecular force spectroscopy. (Springer, 2008)). Notably, effects of the continuous AC magnetic field depend more specifically on the MNP concentration inside the cells and lysosomes while exposure to the pulsed AC magnetic field generates more cell damage at each tested concentration. The exposure to CD sensitizes the cancerous cells to a continuous AC magnetic field, suggesting that less force is generated by continuous exposure. This difference between exposure to continuous and pulsed AC magnetic fields is due to the fact that following the application of force, stress-relaxation processes can occur in the cells, thus shifting the system to a non-equilibrium condition. The multiple pulses and additional application of the force in the non-equilibrium system causes more damage than continuous application.

Our results demonstrate that polymer coats can enhance the intracellular uptake of MNPs and allow subsequent magneto-mechanical actuation of these nanoparticles through the use of super low frequency AC magnetic fields. The work demonstrates that cytoskeletal disruption and subsequent cell death can be selectively enacted upon cancerous cells while leaving healthy cells intact. This type of system which allows for enhanced intracellular uptake, remotely controlled actuation and most importantly cancer cell selectivity has a high impact potential for cancer therapy and could serve as a platform technology in other biomedical applications.

### Example 5

### Magnetic Field Responsive MNPCs for Cancer Theranostics Based on Interconnected Polymeric Micelles and MNPs

Materials:

Reagents and monomers for polymer synthesis, dopamine hydrochloride, benzyl alcohol, iron(III) acetylacetonate (Fe(acac)₃), rhodamine 123 (R123), nitric acid (TraceSELECT), inductively coupled plasma mass spectrometry (ICP-MS) grade standards for iron (Fluka), and MTT were purchased from Sigma-Aldrich Inc., (St. Louis, Mo., USA). PTX was purchased from LC Laboratories (Woburn, Mass., USA). Dissuccinimidyl suberate (DSS), acetonitrile High Performance Liquid Chromatography (HPLC) grade, anhydrous methanol (MeOH), anhydrous DMF, DMSO, PBS and all other HPLC grade of solvents were purchased from Fisher Scientific Inc. (Fairlawn, N.J., USA). Lysotracker® Red-DND 99, Hoechst 33342, Oregon Green® 488-conjugated PTX, and Alex Fluor® (AF) 647-N-hydroxysuccinimide (NHS) were purchased from Life Technologies (Carlsbad, Calif., USA). All cell culture-related materials were purchased from Gibco (Gaithersburg, Md., USA).

Synthesis of Poly(2-Oxazoline) Block Copolymers:

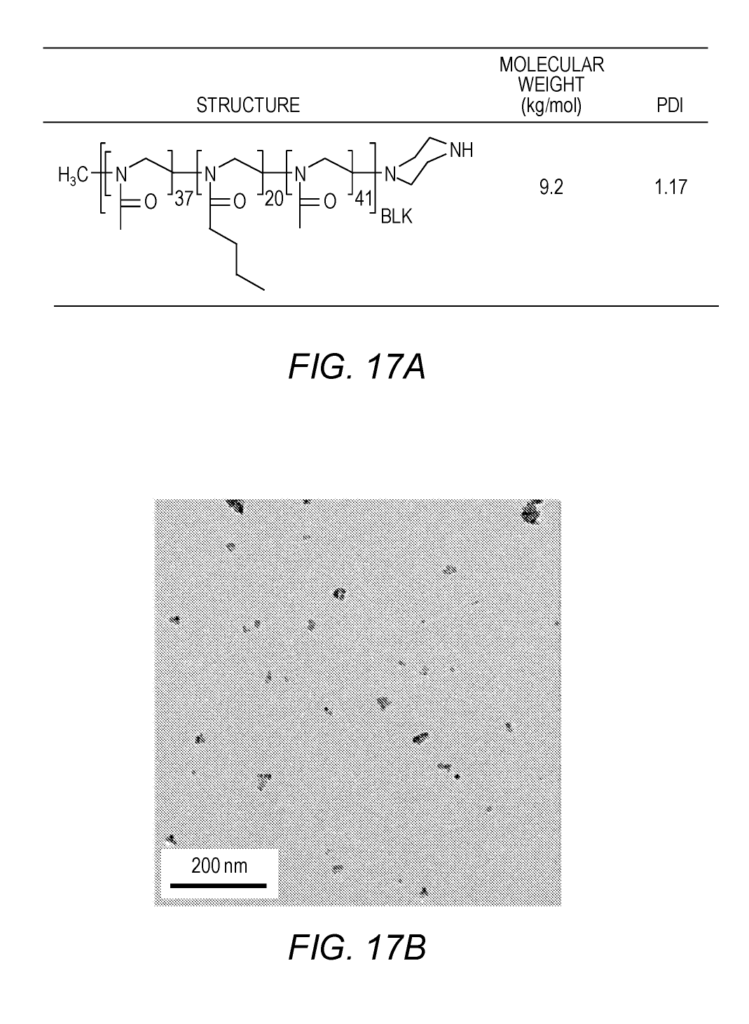

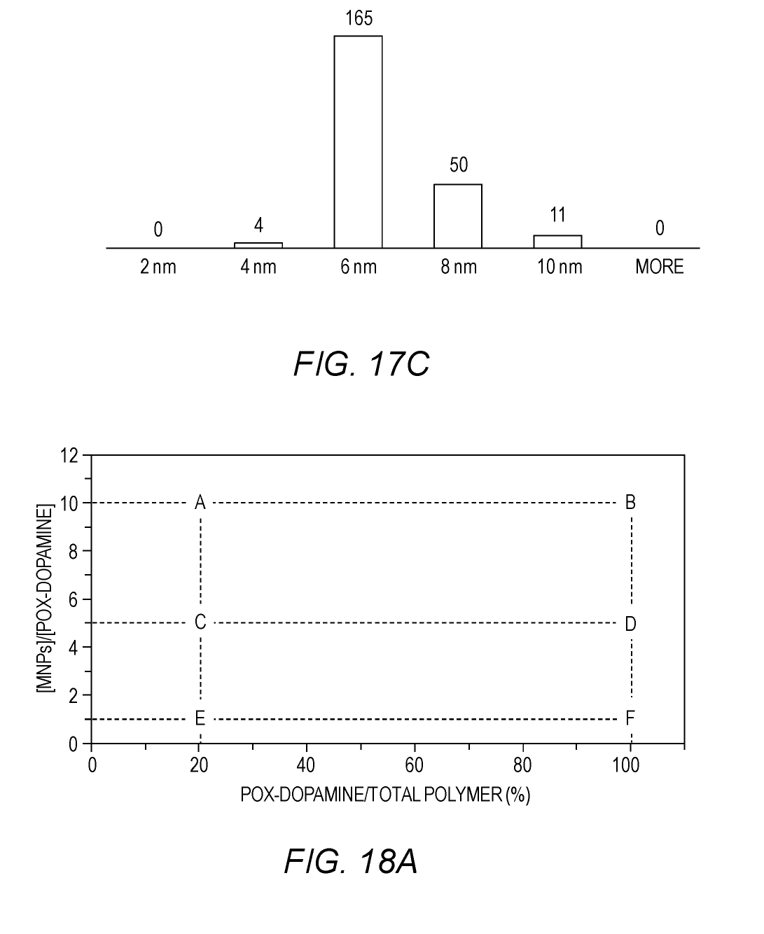

A triblock copolymer of poly(2-butyl-2-oxazoline) (PBuOx) as the hydrophobic block and poly(2-methyl-2-oxazoline) (PMeOx) as the hydrophilic block having PMeOx-b-PBuOx-b-PMeOx structure was used as the poly(2-oxazoline) copolymer in this example. The amphiphilic triblock copolymer was synthesized by the living cationic ring opening polymerization as described previously (Luxenhofer et al., Biomaterials 31:4972 (2010)). All substances, such as monomers, initiators, were refluxed with CaH₂, and distilled under inert argon. The chemical structure, molar mass, and polydispersity of synthesized polymer are presented in FIG. 17.

Synthesis of Dopamine-Conjugated Poly(2-Oxazoline) Copolymer:

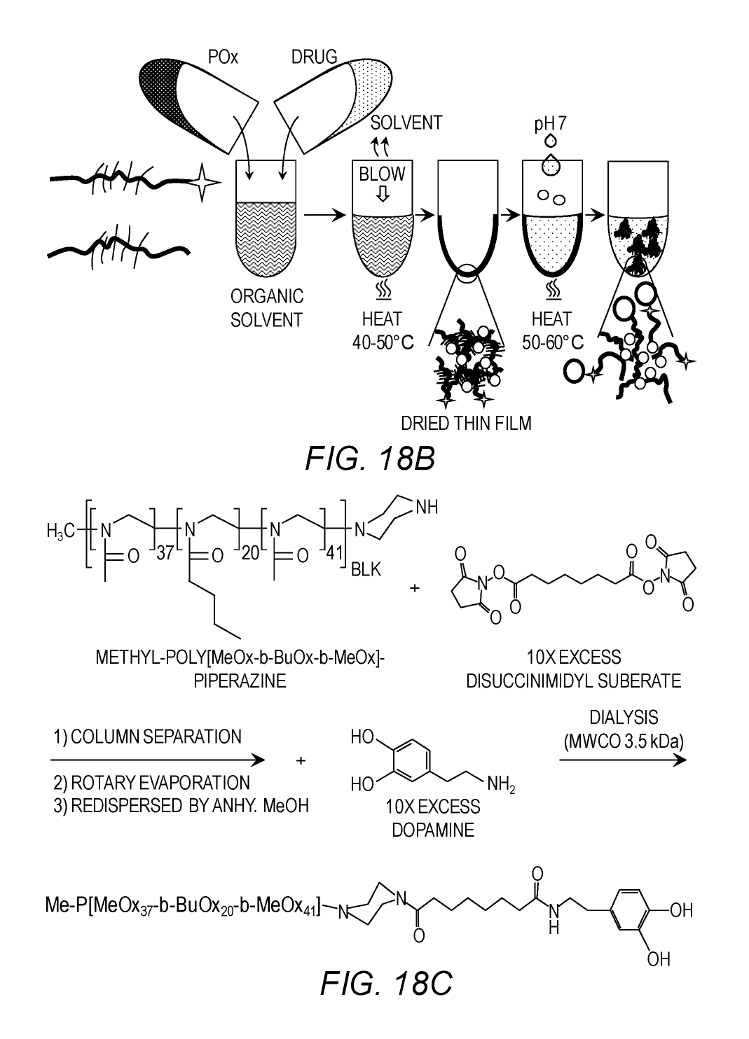

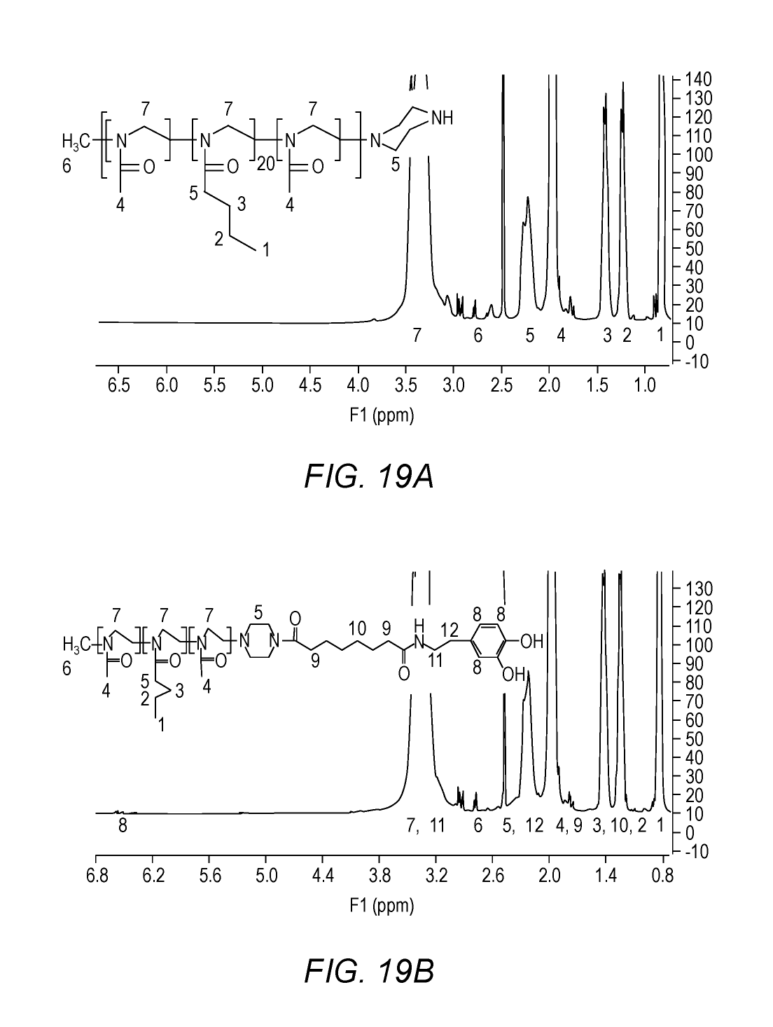

To incorporate MNPs into the theranostic MNPCs (also called here nanoferrogels), the polymer needs to be modified with an anchor group. Dopamine has a high potential to serve as an anchor group, as it combines high affinity to MNPs surface and a presence of a free amine group can be conjugated to polymer chain using various conjugation techniques known in the art. For enhanced cancer theranostic systems, high loading of anticancer drug in the formulation is necessary to decrease excipients-based side effects. In this example, dopamine-decorated poly(2-oxazoline) block copolymer based polymeric micelles were used as the delivery vehicle for a chemotherapeutic drug, PTX, in conjunction with MNPs to which these micelles were attached. Amphiphilic triblock copolymer [Me-PMeOx-b-PBuOx-b-PMeOx-piperazine)—molecular weight 9,200 g mol⁻¹, and polymer polydispersity index (Mw/Mn)=1.17—was employed for the micelle preparation (FIG. 17A). A dopamine anchor group was conjugated to this copolymer using DSS—an amine selective linker (FIG. 18C). ¹H NMR showed that essentially 100% of polymer chains were successfully modified with dopamine (FIG. 19). The procedure of dopamine conjugation to poly(2-oxazoline) block copolymer was modified from Tong et al. (Mol. Pharm. 7:984 (2010)). Briefly, piperazine-terminated poly(2-oxazoline) was dissolved in anhydrous MeOH, and mixed with a 10-fold molar excess amount of DSS in anhydrous DMF. Sodium borate buffer (0.1 M, pH8.0) was added to the mixture, and incubated for 1 h at room temperature at constant magnetic bar stirring at around 400 rpm. Free DSS was removed by gel filtration (Sephadex LH-20 column) in anhydrous MeOH. Activated poly(2-oxazoline)-DSS was collected, and the solvent was removed in vacuo. A 20-fold molar excess of dopamine was dissolved in anhydrous MeOH, mixed with the activated poly(2-oxazoline)-DSS and left overnight at 4° C. Excess of dopamine and other impurities were removed by the dialysis (MWCO 20 kDa).

Synthesis of MNPs:

MNPs were synthesized by thermal decomposition of Fe(acac)₃ in anhydrous benzyl alcohol as described by Pinna et al. (Chem. Materials 17:3044 (2005)), with minor modifications (Vishwasrao et al., Chem. Materials 28:3024 (2016)). Two methods are commonly used for MNPs synthesis, co-precipitation of ferrous and ferric ions in the presence of a base, e.g., in an alkali aqueous solution (Massart, IEEE Transactions on Magnetics 17:1247 (1981)) and the thermal decomposition of iron precursor. Although the MNPs formed by the co-precipitation method have a hydrophilic surface and can be dispersed in aqueous media, it is difficult to control the size and size distribution of the formed MNPs. The thermal decomposition of an organic iron precursor in non-aqueous solvent, such as benzyl ether, or benzyl alcohol allows better control of particles size. For example, we have reported that size and size distribution of MNPs could be narrowly controlled and tuned by introducing small changes in the heating sequence (Vishwasrao et al., Chem. Materials 28:3024 (2016)). In this example, briefly, 6.2 μmols of Fe(acac)₃ was mixed in three-necked flask with 45 mL of anhydrous benzyl alcohol. The reaction mixture was heated up to 110° C. and incubated for 1 hr to remove moisture. Once the moisture was completely removed, temperature was gradually increased to 205° C. at a rate of approximately 2° C. min⁻¹, and the mixture was incubated at 205° C. for 40 hr. The formed MNPs were precipitated and washed by acetone using decantation with a magnet, and residual organic solvent was completely evaporated in the rotary evaporator. The MNPs were characterized by TEM and superconducting quantum interference device—vibrating sample magnetometer (SQUID-VSM) (Quantum Design Co.) to determine their size distribution, and magnetization saturation, respectively. The results are presented in FIGS. 17B and 17C. In this study, we have further selected on narrowly-dispersed MNPs with a diameter of 5.5±1.1 nm for subsequent synthesis of MNPCs.

Preparation of PTX-Containing MNPCs:

In this example PTX-loaded MNPCs were synthesized in 2 steps. First, the PTX/Poly(2-oxazoline) micelles were prepared. Second, the MNPs were incorporated to the network of PTX-loaded micelles (FIG. 18B). MNPs prepared by thermal decomposition can be dispersed in aqueous media, and coated with hydrophilic polymers, to form clusters that can be loaded with drugs. Specifically, the MNPs can be dispersed in alkaline water (pH 12), and mixed with the polymer solution. However, due to poor stability of PTX in alkaline environment caused by possible hydrolysis of ester groups as well as decrease low binding affinity of dopamine to MNPs surface caused by oxidation to dopaquinone, the pH of the solution was kept neutral (pH 7.4). PTX-loaded polymeric micelles were prepared by the film hydration method (Luxenhofer et al., Biomaterials 31:4972 (2010)). Briefly, predetermined amounts of polymer (poly(2-oxazoline) and/or poly(2-oxazoline)-DSS-dopamine) and PTX were dissolved by ethanol, and the organic solution was removed using airflow upon heating (45° C.). To remove residual ethanol, the sample was kept in vacuo overnight. Once a dried film was formed, warm deionized water (DI water) was added to it followed by mild agitation of the dispersion at 60° C. incubation for 20 min. The polymeric micelle solution was cooled down to room temperature, and centrifuged at 10,000 rpm for 3 min to remove unloaded PTX. The dispersion of 0.5 mg mL⁻¹ of MNPs was sonicated (500 V, 2 kHz, 20% power, 10 seconds on, 5 seconds off) for 30 min to avoid aggregation of bare MNPs, and then drop-wise added to the dopamine-decorated polymeric micelles. The resulting mixture was kept for at least 12 h upon magnetic stirring at around 400 rpm. Purification of PTX-loaded MNPCs was done by gel filtration (Sephadex G-25 column, DI water as eluent), and the samples were lyophilized.

The stability and physicochemical properties of PTX-loaded MNPCs were affected by several factors including: 1) molar ratio of MNPs to poly(2-oxazoline)-DSS-dopamine, 2) % dopamine conjugated polymer in the overall poly(2-oxazoline) used for the micelle preparation, and 3) loading of PTX in poly(2-oxazoline) micelles. We designed 6 different formulations (FIG. 18A). First, the three different stoichiometric ratios of [MNPs]/[poly(2-oxazoline)-DSS-dopamine]⁻¹ were set: 10 (PTX-A, PTX-B), 5 (PTX-C, PTX-D), and 1 (PTX-E, PTX-F). Second, the degree of dopamine conjugation was set to either 20% (PTX-A; PTX-C; PTX-E) or 100% (PTX-B; PTX-D; PTX-F). The actual feeding amounts of drug, polymer, and MNPs for these compositions are presented in Table 2. The use of poly(2-oxazoline) block copolymer in this MNPC formulations allowed us to set a very high PTX/polymer feeding ratio of 1:5, and due to the very high loading capacity of poly(2-oxazoline) polymeric micelles as reported previously (Seo et al., Polymers Adv. Technol. 26:837 (2015)) and even higher feeding ratios of up to 1:1 wt. can be achieved. These feeding ratios greatly exceed those that were previously reported that were less than 1:10—i.e., all were less than 10 wt % drug loading, (Cui et al., Biomaterials 34:8511 (2013); Dilnawaz et al., Biomaterials 33:2936 (2012); Jain et al., Biomaterials 29:4012 (2008); Schleich et al., J. Controlled Release 194:82 (2014); Zavisova et al., J. Magnetism Magnetic Materials 321:1613 (2009)). suggesting major improvement of the MNPCs of this invention compared to polymeric nanoparticles known in the art.

TABLE 2
Feeding amount and composition of PTX MNPCs

| | Feeding amount (mg) | | | | Composition | | | |
|---|---|---|---|---|---|---|---|---|
| | | Poly(2- | | | PTX | MNP | | |
| | Poly(2- | oxazoline)- | | | (wt | (wt | LE¹ (%) | |
| Formulation | oxazoline) | Dopamine | MNPs | PTX | %) | %) | PTX | MNP |
| PTX-A | 8 | 2 | 0.5 | 2 | 17.1 | 2.7 | 106.9 | 67.5 |
| PTX-B | 0 | 10 | 2.4 | 2 | 13.5 | 11.9 | 97.9 | 69.0 |
| PTX-C | 8 | 2 | 0.24 | 2 | 14.4 | 1.2 | 88.2 | 68.8 |
| PTX-D | 0 | 10 | 1.2 | 2 | 15.1 | 6.7 | 100 | 71.0 |
| PTX-E | 8 | 2 | 0.05 | 2 | 15.9 | 0.3 | 95.8 | 72.3 |
| PTX-F | 0 | 10 | 0.24 | 2 | 16.7 | 1.3 | 102.3 | 63.7 |

¹LE (wt %) was calculated as the amount of PTX (or MNP) in final formulation/the input amount of PTX (or MNP) × 100.

Physicochemical Characterization of PTX-Containing Polymeric Micelles and MNPCs:

The amount of PTX entrapped in the polymeric micelles and MNPCs was quantified by high performance liquid chromatography (HPLC) system (Agilent Technologies 1200 Series, 250 mm×4.6 mm Phenomex C18-5 μm column). The samples were diluted by acetonitrile, centrifuged at 12,000 rpm for 60 min, and supernatant was collected under magnet decantation, and injected (20 μL) into HPLC system. The mobile phase was composed of acetonitrile and H₂O (55:45 v/v), the flow rate was 1.0 mL min⁻¹, the column temperature was set to 40° C., and the detection wavelength was 227 nm. The retention time for PTX was 6 min. The size of dopamine conjugated poly(2-oxazoline) micelles was determined by DLS using a Zetasizer Nano ZS (Malvern Instruments Ltd., Malvern, UK). The particle size of PTX-loaded polymeric micelles MNPCs was determined by the Nanoparticle Tracking Analysis (NTA) using Nanosight instrument equipped with the NTA 2.0 analytical software (Nanosight NS500, Wiltshire, United Kingdom). The MNPC formulations were prepared at concentration 50 μg mL⁻¹ for setting approximate particle concentration at 10⁸ particles mL⁻¹. A 60 sec video was recorded and analyzed by NTA software. The zeta-potentials were determined by DLS using a Malvern Zetasizer (Malvern Instruments, Malvern, UK). The formulations were diluted to 1.0 mg mL⁻¹ in DI water, and placed in disposable zeta cells for measurements. The TEM images of the uncoated MNPs, and PTX-MNPCs were taken by JEOL 2010F-FasTEM (Peabody, Mass., USA). The samples were diluted to approximately 0.25 mg mL⁻¹ in DI water. A drop of diluted sample was put onto a carbon-coated cupper grid (TedPella Inc., Redding, Calif., USA) and dried in air. A drop of 5% uranyl acetate was added on the TEM grid for negative staining. The particle size and size distribution of uncoated MNPs were calculated by ImageJ software Saturation magnetization measurements. The saturation magnetization was measured by SQUID-VSM (Quantum Design Co.) at 300° K. Pre-weighted samples were placed in sample holder, and mounted in a transparent straw.

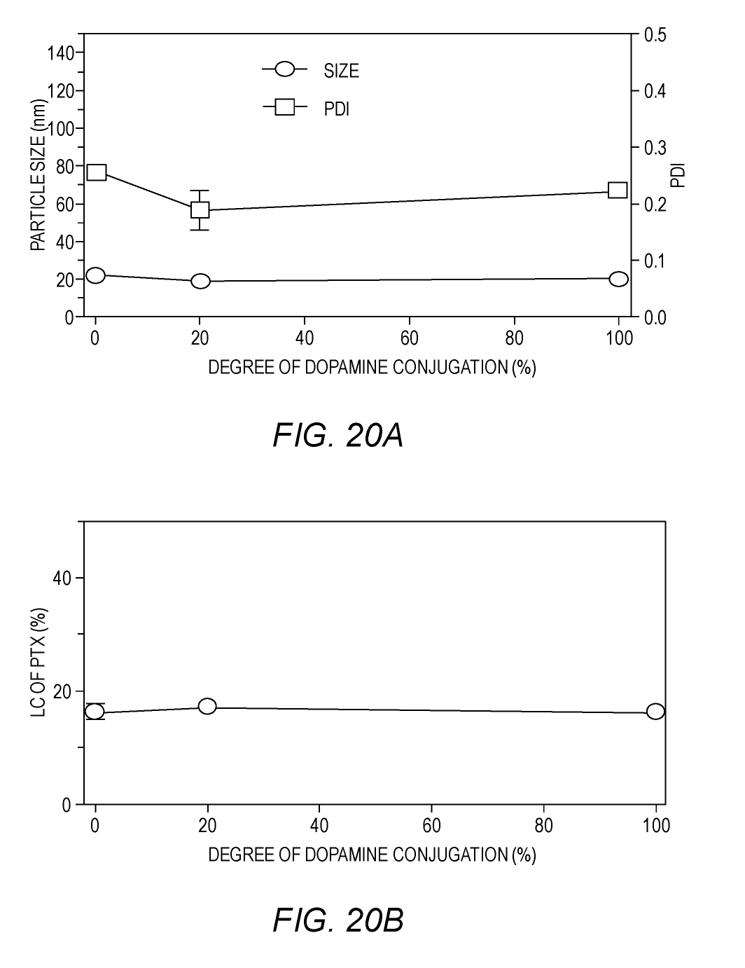

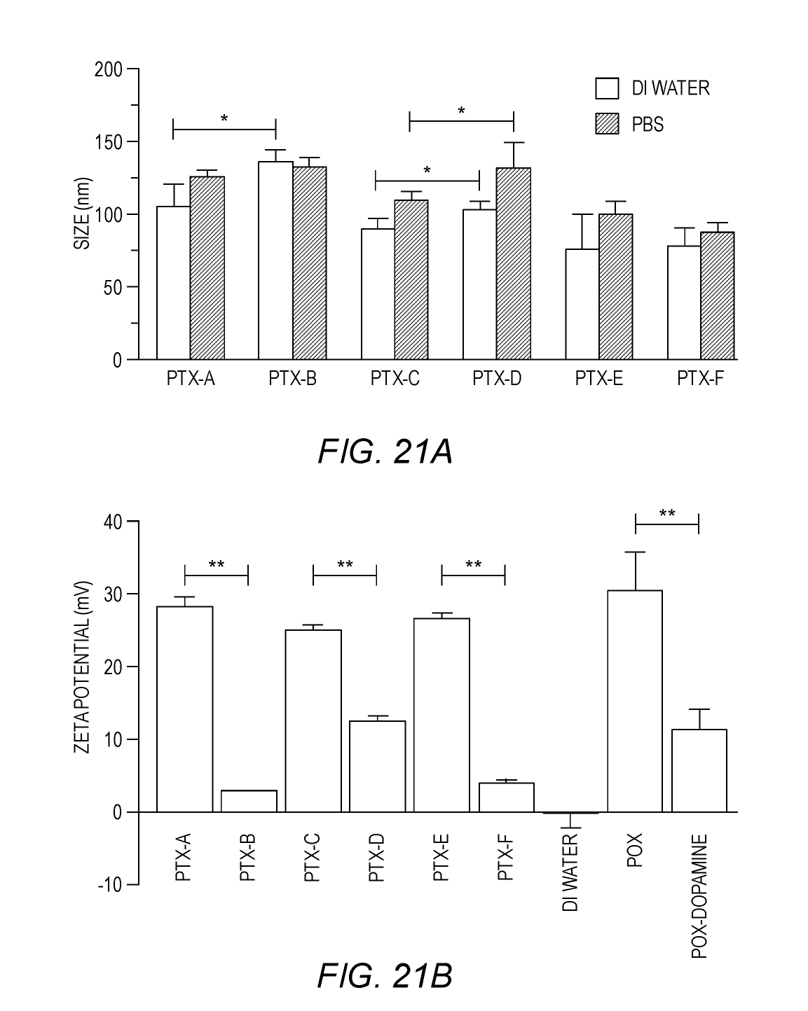

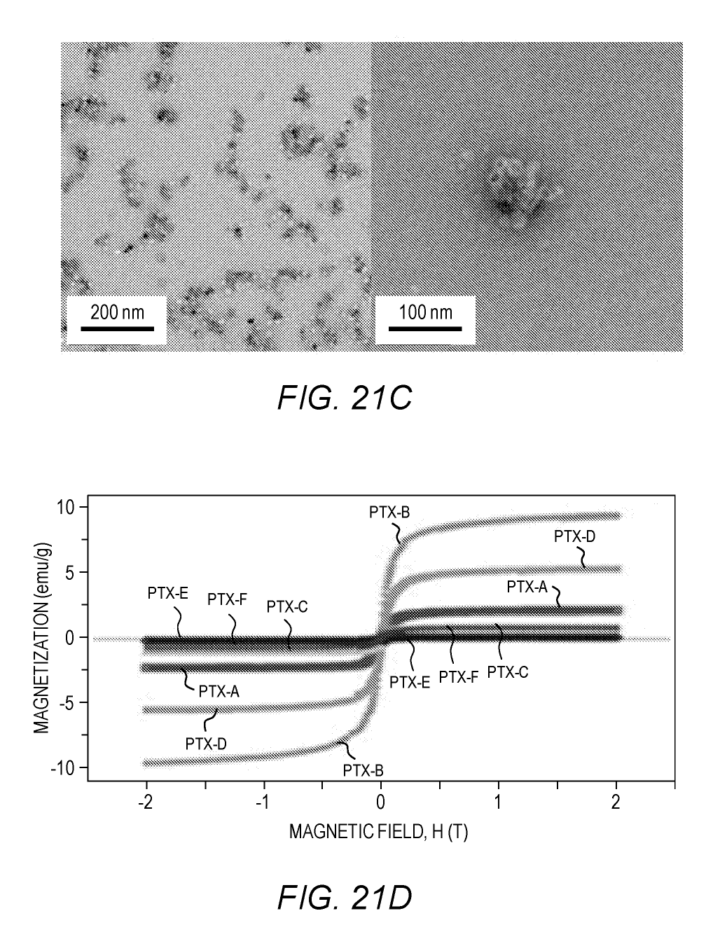

The results demonstrate that the dopamine conjugation to the poly(2-oxazoline) polymeric micelles did not affect physicochemical properties of these micelles DLS particle size, PDI, and loading capacity of PTX (FIG. 20). The hydrodynamic diameters of PTX-loaded MNPCs in various media measuring by NTA are shown displayed in FIG. 21A. The molar ratio of [MNPs]/[Poly(2-oxazoline)-dopamine]⁻¹ affected the size: as the MNP content increased the particle size also increased. When measured in DI water, the averaged-size of PTX-A, and PTX-B formulations displayed the largest particle size (105 nm and 135 nm, respectively), PTX-E, and PTX-F formulations displayed the smallest size (89 nm and 78 nm, respectively), and the particle sizes of the PTX-C and PTX-D formulations were in the middle range (89 nm and 103 nm). A similar trend was also revealed in PBS as the MNP content increased the particle size also increased. In the absence of dopamine the surface charge of poly(2-oxazoline) micelles due to the presence of terminal piperazine groups was positive as was evident from the high positive zeta-potential values of these micelles (30.5 mV, FIG. 21B). However, when piperazine was chemically linked via DSS linker to dopamine, the zeta potential of the polymeric micelles was significantly decreased to 11.5 mV. Similar trend was observed for the MNPCs as the MNPC formulations at the 20% of poly(2-oxazoline)-DSS-dopamine content in the poly(2-oxazoline) (PTX-A; -C; -E) all had higher zeta potentials compared to the MNPCs formulations comprising 100% of poly(2-oxazoline)-DSS-dopamine and no unconjugated polymer (PTX-B; -D; -F). The morphology of PTX-loaded MNPCs analyzed by measured by TEM (FIG. 21C) suggested that MNPs were surrounded by poly(2-oxazoline) micelles, forming raspberry-like clusters. The TEM sizes were generally consistent with those determined by NTA. The PTX-loaded MNPCs exhibited superparamagnetic behavior without any remnant magnetization: their magnetization was increased as the magnetic field increased (FIG. 21D). The saturation of magnetization (Ms) depended on the content of iron oxide in the formulations. After surface modification of MNPs by attaching polymeric micelles the Ms values decreased compared to uncoated bare MNPs. The decrease in Ms was probably attributable to the interaction of magnetite with the dopamine-anchored polymer chains at the surface of the MNPs (Yuan et al., Langmuir 28:13051 (2012); Rahimi et al., J. Nanosci. Nanotechnol. 9:4128 (2009)). The PTX-loaded MNPC B had the highest Ms, while the PTX-loaded MNPC E had the lowest Ms (9.5, and 0.15 emu/g, respectively).

Effect of Magnetic Field Exposure on the Cell Viability:

Briefly, MCF-7, MDA-MB-231, LCC-6-WT, LCC-6-MDR, and BT474 cells were seeded at 5,000 cells well⁻¹ in 2×8 MICROLON 96 well high binding strips (Griener Bio Inc., Monroe, N.C.), and let to adhere for 3 days. Cells were treated with various concentrations of MNPCs for 24 h at 37° C., and washed by PBS. Cell strips were placed in magnetic field generator, and continuously exposed to AC magnetic field for 30 min with 50 Hz and 50 kA min. For the pulsed field exposure, cells were exposed to the same field for 10 min, and then left for 5 min without field for repeating cycles so that the total exposure period was 30 min. After the field exposures, the cells were incubated at 37° C. for 4 h, and the cytotoxicity was determined by the standard MTT assay. All statistical comparisons were carried out using Graphpad Prism. Comparison between groups was done by Student's t test, or ANOVA with Dunnect's post hoc test for multiple comparisons.

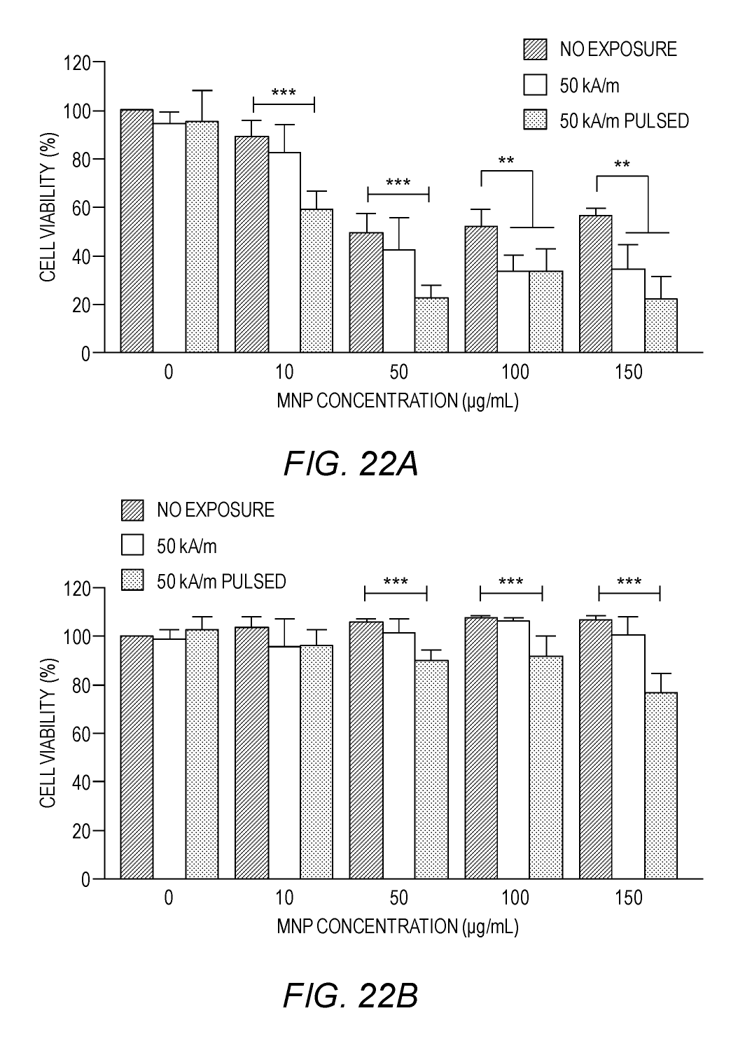

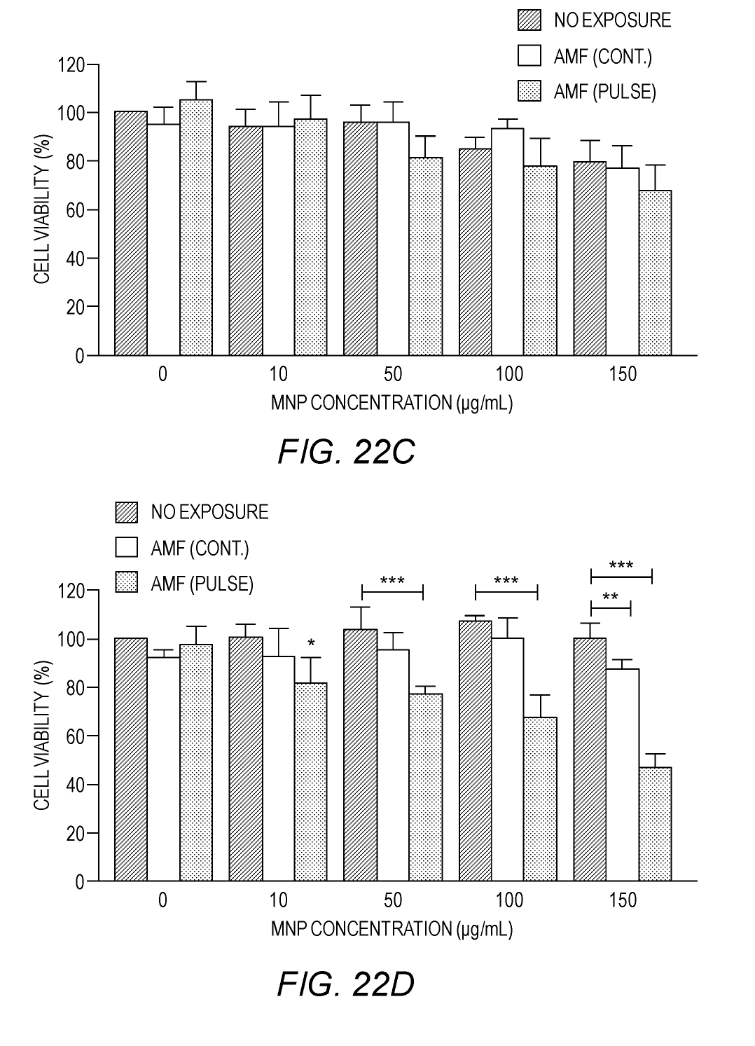

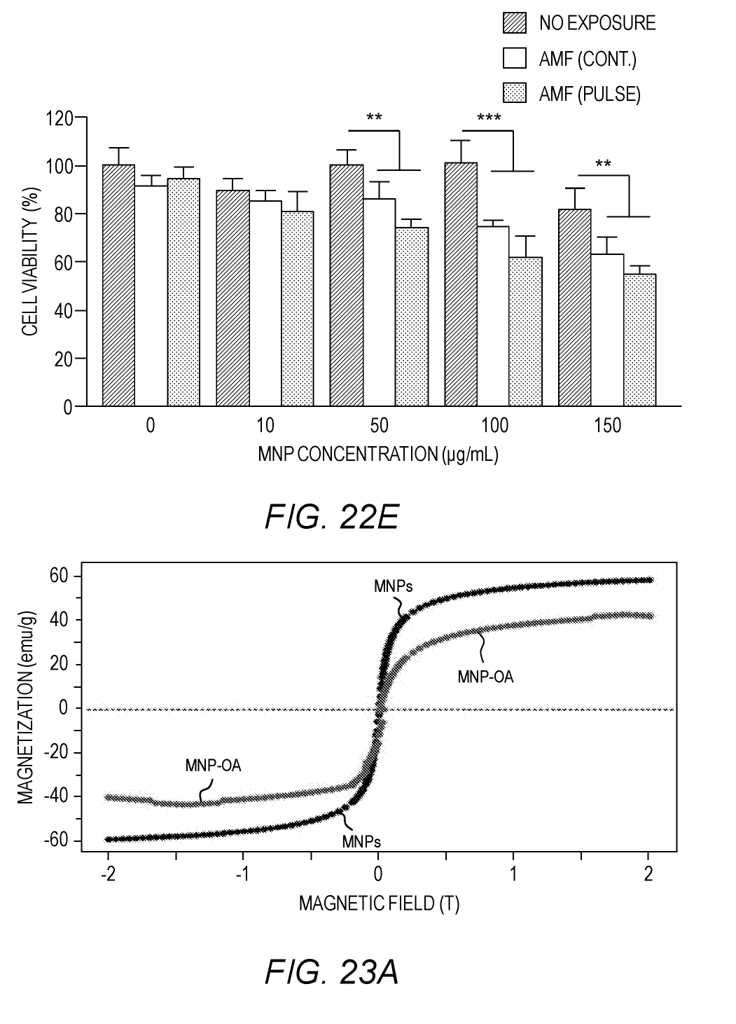

In LCC-6-WT and triple negative breast cancer cells MDA-MB-231 the cells viability was significantly reduced when cells were exposed to AC magnetic field (FIGS. 22 A and 22E). There was no significant difference in cytotoxic effects between AC magnetic field treatment groups when MNP concentration was higher than 100 μg/mL (FIG. 22A). Also, in P-gp overexpressing cells, LCC-6-MDR, significant cytotoxic effects were only observed under pulsed AC magnetic field regime (FIG. 22B). In contrast, no significant field-induced cytotoxicity was observed in MCF-7 cells (FIG. 22C). In BT-474 cells, pulsed AC magnetic field was significantly more effective compared to continuous AC magnetic field (FIG. 22D). Cell viability after exposure to 150 μg/mL MNPCs was significantly decreased from 100% to 46% when cells were exposed to pulsed AC magnetic field. Overall, the results suggest that treatment of the cells with MNPCs followed by the field exposure increased toxicity to cancer cells compared to no field treatments, and that the pulsed field exposure has a greater cytotoxic effect than the continuous field exposure.

### Example 6

### Magnetic Field Responsive MNPCs for Cancer Theranostics Based on Hydrophobically Modified MNPs Dispersed by Amphiphilic Block Copolymers

Synthesis of Poly (2-oxazoline)s:

The amphiphilic poly (2-oxazoline) triblock copolymers were synthesized by the living cationic ring opening polymerization as described in Example 5. All substances, such as monomers, initiators, were used under reflux with CaH₂, and distilled under inert argon. The synthesized polymer [Me-PMeOx-b-PBuOx-b-PMeOx-piperazine)] had a molecular weight of 9,200 g mol⁻¹, and polymer polydispersity index (Mw/Mn)=1.17.

Synthesis of MNPs and Oleic Acid Coated MNPs (MNP-OA):

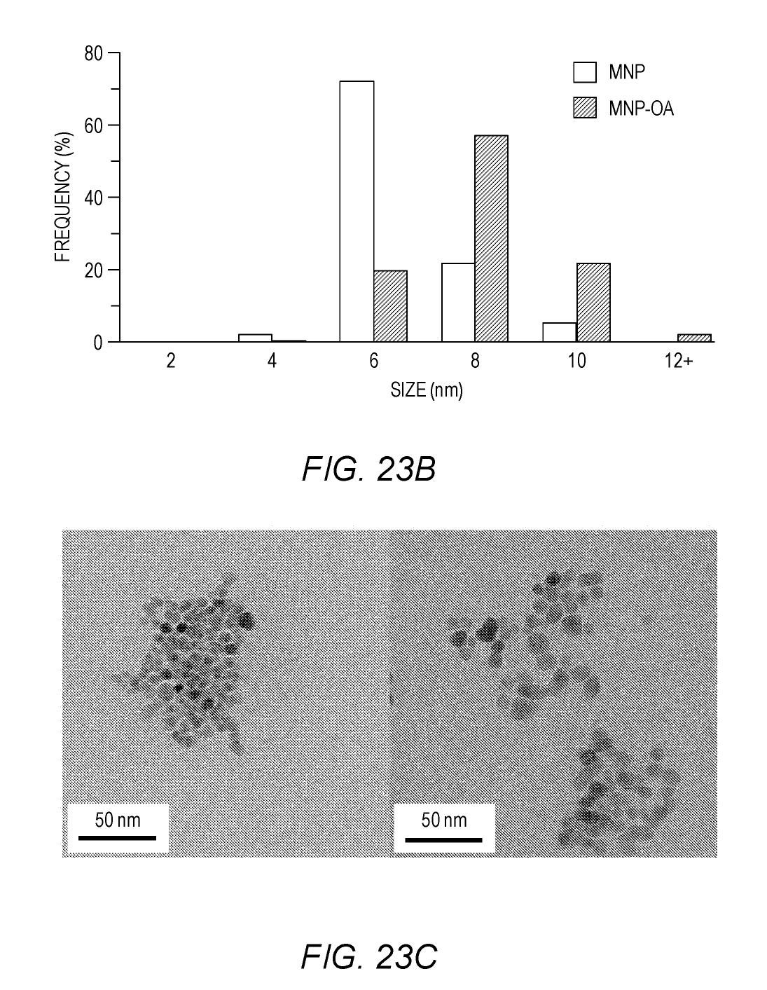

MNPs were synthesized by thermal decomposition of Fe(acac)₃ in anhydrous benzyl alcohol as described in Example 5. Briefly, 6.2 mols of Fe(acac)₃ was placed in three-necked flask with 45 mL of anhydrous benzyl alcohol. The reaction mixture was heated up to 110° C. for 1 hr to completely evaporate any moisture. Once moisture was removed, the temperature was gradually increased to 205° C. at a rate of approximately 2° C. min⁻¹, and kept constant for 40 hr. MNPs were washed by acetone under magnet decantation, and residual organic solvent was completely evaporated using a rotary evaporator. In order to coat the surface of MNPs with the oleic acid, 100 mg of MNPs were dispersed in methanol, and heated to 85° C. upon 400 rpm magnetic stirring. A 10-fold molar excess of oleic acid was added drop-wise to the MNPs dispersion, and then the solvent was evaporated. In order to remove free oleic acid, MNP-OA substance was washed by acetone using nanoparticles decantation with a magnet, and the residual organic solvent was completely removed in a rotary evaporator. The MNPs and MNP-OA were characterized by TEM, and SQUID-VSM (Quantum Design Co.) as described above to determine their size distribution, and magnetization saturation, respectively. The results are presented in FIG. 23.

Preparation of MNPCs:

The PTX loaded MNPCs comprising MNP-OA and poly(2-oxazoline) polymeric micelles were prepared by the film hydration method (Luxenhofer et al., Biomaterials 31:4972 (2010)). Briefly, a predetermined amount of Me-PMeOx-b-PBuOx-b-PMeOx-piperazine and PTX dissolved in ethanol and MNP-OA dispersed in chloroform (MNP-OA), were mixed at a poly(2-oxazoline):PTX:MNP-OA ratio 10:2:1. The bulk of the organic solvents was then gently removed via airflow upon slight heating (45° C.) and then the sample was kept in vacuo overnight in order to remove the residual organic solvent. The warm deionized water (DI water) was added to the obtained dried film upon gentle agitation at 60° C. for 20 min. The resulting dispersion was cooled down to room temperature, and filtered using a 0.45 m syringe filter to remove unloaded PTX and MNP-OA. A 10-fold molar excess of bis(sulfosuccinimidyl)suberate (BS3) amine-to-amine cross-linker dissolved in DI water, was added to this dispersion drop-wise and the dispersion was kept upon magnetic stirring for at least 12 h. The resulting PTX-MNPCs were purified by gel filtration on a Sephadex G-25 column equilibrated with DI water, and the samples were lyophilized. The PTX and MNP contents in the MNPC were determined by HPLC and ICP-MS, respectively. The feeding amount and the resulting composition are summarized in Table 3.

TABLE 3
Feeding amount and composition of type-B PTX MNPC

| Feeding amount (mg) | | | | Composition ¹ | |
|---|---|---|---|---|---|
| | Poly | | | PTX | MNP |
| Formulation | (2-oxazoline) | PTX | MNP-OA | (wt %) | (wt %) |
| | 10 | 2 | 1 | 6.4 | 5.1 |

¹ The composition was calculated as the weight percent by dividing the amount of PTX or MNP found in the final formulation dividing by the total weight of the final formulation × 100%.

Physicochemical Characterization of PTX-MNPCs:

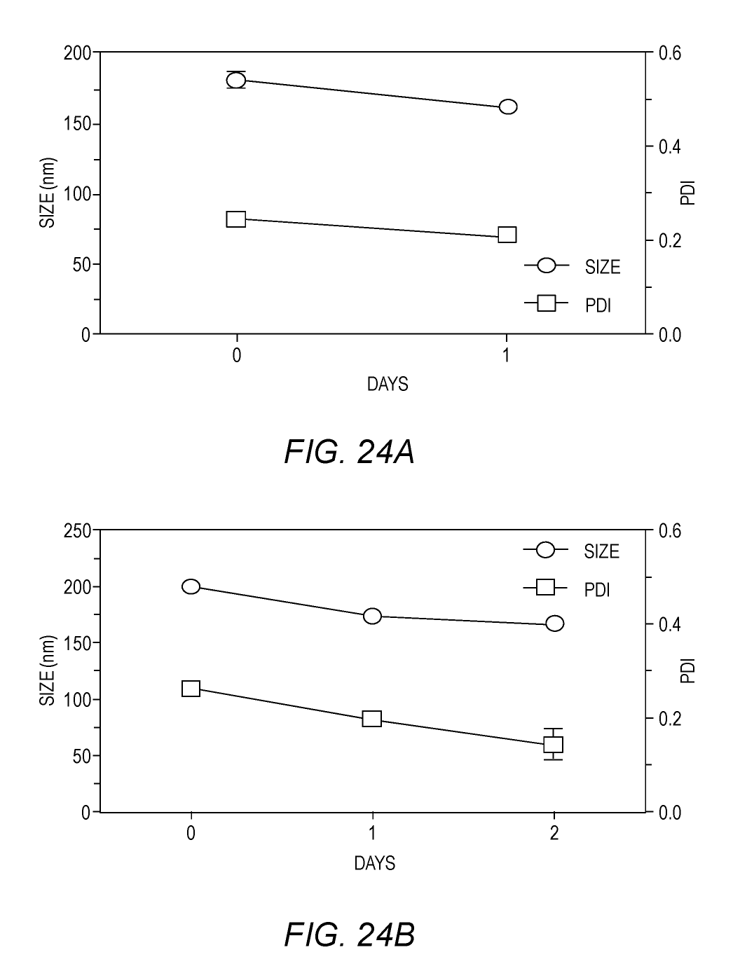

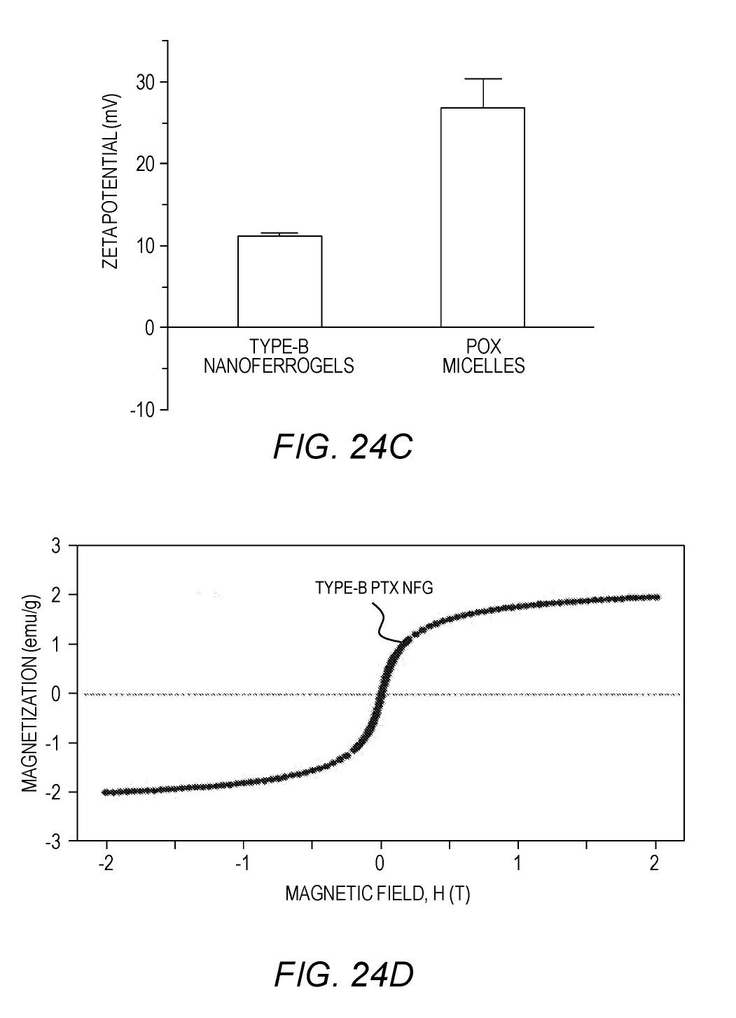

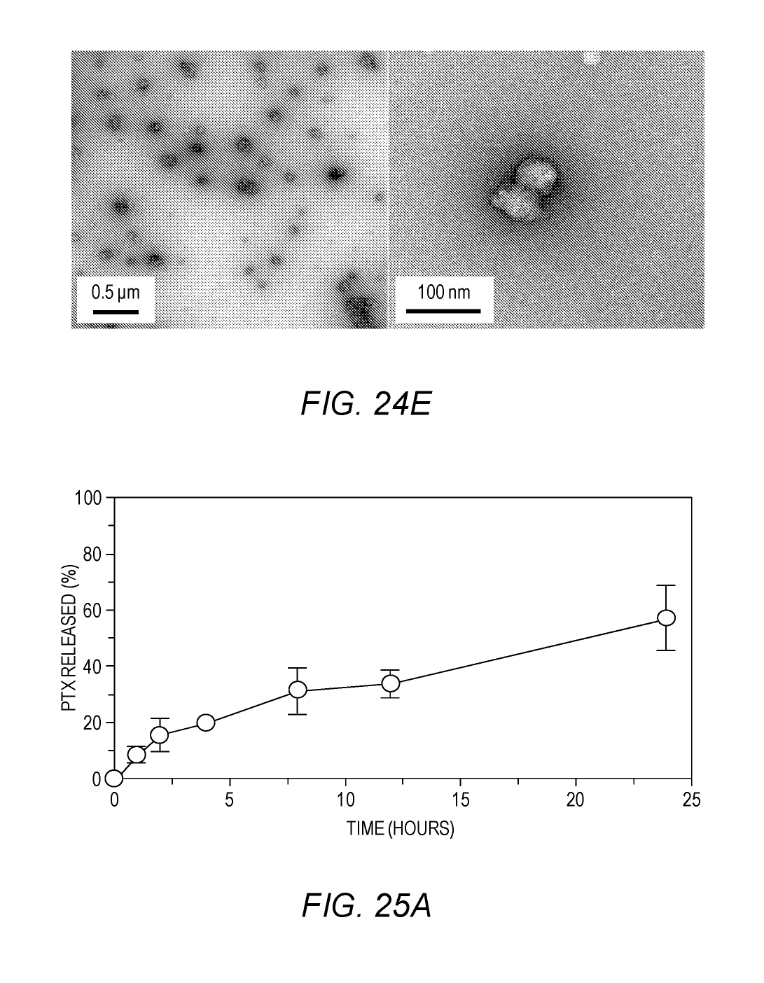

The amount of PTX in formulation was quantified by HPLC (Agilent Technologies 1200 Series, 250 mm×4.6 mm Phenomex C18-5 μm column). The samples dispersed in acetonitrile were centrifuged at 12,000 rpm for 20 min, and supernatant was injected (20 μL) into the HPLC system. The mobile phase was composed of acetonitrile and H₂O (55:45 v/v), the flow rate of mobile phase was 1.0 mL min⁻¹ and the column temperature—40° C. The UV detection was carried out at wavelength 227 nm. The retention time for PTX was 6 min. The particle size of PTX MNPCs and zeta potential were measured by DLS using a Zetasizer Nano ZS (Malvern Instruments Ltd., Malvern, UK). The samples were diluted by DI water to 1.0 mg mL⁻¹, and inserted into disposable zeta cells for measurements. TEM images of the uncoated MNPs, MNP-OA, and PTX MNPCs were taken by JEOL 2010F-FasTEM (Peabody, Mass., USA). The samples were diluted to approximately 0.25 mg mL⁻¹ in DI water. A drop of diluted sample was put onto a carbon-coated cupper grid (TedPella Inc., Redding, Calif., USA) and dried. Prior to TEM a drop of 5% uranyl acetate was added on the TEM grid for negative staining. The saturation magnetization was determined by SQUID-VSM (Quantum Design Co.) at 300° K. Pre-weighted samples were placed in sample holder, and mounted in a transparent straw. The results of the characterization are presented in FIG. 24.

PTX Release Studies:

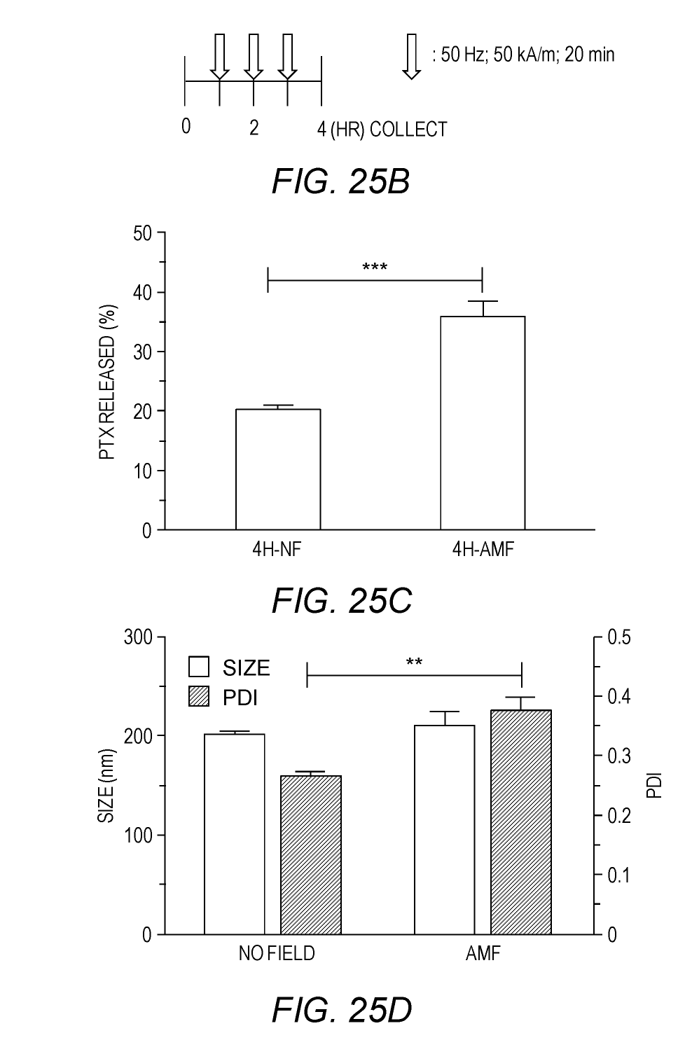

The in vitro release rates of PTX from the PTX MNPCs formulation were determined by the dialysis method. Each formulation was diluted with the release media containing 40 g/L bovine serum albumin (BSA) to yield concentration of formulation 0.5 mg mL⁻¹ (PTX concentration was approximately 50 μg mL¹). Then 100 μL of diluted samples were placed in dialysis device (Slide-A-Lyzer, 20 kDa MWCO, Thermo Scientific), and introduced in 20 mL of various media at 37° C. under shaker (50 rpm). When applying AC magnetic field, samples were inserted to MF generator (Nanomaterials Ltd., Tambow, Russia) and treated for 20 min at a frequency of 50 Hz and field strength of 50 kA/m. At predetermined time point, each sample was removed from the dialysis tube and analyzed by HPLC to determine PTX concentration. The results are presented in FIG. 25 and demonstrate that the treatment with the magnetic field increase drug release (FIG. 25C) and induce changes in particle size polydispersity (FIG. 25D).

Effect of Magnetic Field Exposure on Cell Viability:

Briefly, non-cancerous MCF-10A cells, and cancer MDA-MB-231 and BT474 cells were seeded at 5,000 cells well⁻¹ in 2×8 MICROLON 96 well high binding strips (Griener Bio Inc., Monroe, N.C.), and let to adhere for 3 days. Cells were treated with various concentrations of MNPCs synthesized in this example for 24 h at 37° C., and washed with PBS. Cell strips were placed in a magnetic field generator, and continuously exposed to AC magnetic field for 30 min with 50 Hz and 50 kA m⁻¹. For the pulsed field exposure, cells were exposed to the same field for 10 min, and then left for 5 min without field for repeating cycles so that the total exposure period was 30 min. After the field exposures, the cells were incubated at 37° C. for 4 h, and the cytotoxicity was determined by the standard MTT assay.

There was no significant difference in cytotoxic effects between AC magnetic field treatment groups and treatment groups without magnetic field in all three cells lines. Notably, in BT-474 cells, neither continuous nor pulsed AC magnetic field increased cytotoxicity compared to no-field treatment. This was a striking contrast to the results in Example 4 and 5 were the effects of the field treatment in these cancer cells were the greatest. This suggests that the PAA-P85-MNPs MNPCs in Example 4 and nanoferrogels MNPCs in Example 5, in which MNPs are attached to hydrophilic polyion or water-soluble nonionic polymers are more potent in killing cancer cells in response to the field treatment than the hydrophobically-modified MNPs.

### Example 7

### Preparation of MNPC Comprising Amphiphilic Block Copolymers in Aqueous and Non-Aqueous Media

MNPCs are prepared using formulation techniques in aqueous or non-aqueous media. For example, crystalline Fe₃O₄ nanoparticles are synthesized by thermal decomposition of Fe(acac)3 in benzyl ether in the presence of oleic acid. Using this process the resulting magnetite particle size varies from 5-7 nm to 10-15 nm depending on the temperature rate. The oleic acid residues form a temporary coat surrounding the MNPs that is later displaced by the stronger bisphosphonate-ligands in the bisphosphonate-poly(2-oxazoline) block copolymers, a process driven by enthalpy and entropy. The displacement is carried out in a good solvent for both hydrophilic and hydrophobic blocks and, hence, no self-assembly is expected at this stage. In the case of amphiphilic poly(2-oxazoline) block copolymers, hydrophobic host molecules (for example drugs, such as PTX or DTX) are introduced to the polymer-coated MNPCs dispersion to produce drug-containing MNPCs. Removal of organic solvent and subsequent film hydration leads to a polymer-MNP film (with or without blended drugs), which upon hydration spontaneously self-assemble into the individual MNPCs with surface-bound polymeric micelles or MNP-clustered aggregates (nano-ferrogels). Alternatively, MNPCs are produced as micelle-like particles by turbulent mixing of the organic and aqueous phases. The Reynolds numbers (Re) are optimized to minimize particle size distribution (PDI 0.1 to 0.15). For example, Re for Pluronic coated MNP is 7,000 (THF/H₂O).

To achieve MNPs coating in aqueous media the F₃O₄ nanoparticles are synthesized by thermal decomposition of Fe(acac)₃ in benzyl alcohol without addition of oleic acid or other co-surfactants. The MNPs are isolated and then re-dispersed in alkaline water (pH>9) and further mixed with water-soluble alendronate-poly(2-oxazoline) block copolymers by turbulent mixing. Generally, in the case of amphiphilic block copolymers (AB, or ABA) certain co-solvents (e.g., DMSO) are added to create non-selective conditions on otherwise aqueous media. In aqueous media block copolymers form stable micelles with segregated hydrophobic B block cores and hydrophilic A block shells carrying the anchor groups, which can be reacted with MNPs. To alter the number of points of attachment of micelles to MNPs the alendronate-containing copolymers are blended with anchor-free AB, ABA, or ABC copolymers. To solubilize hydrophobic solutes (e.g., drugs) in the cores of the preformed micelles, we first prepare block copolymer blends with these solutes in a common solvent, then evaporate solvent to form thin films, then rehydrate the films to form mixed micelles with incorporated solutes and finally react these micelles with the MNPs in aqueous media.

### Example 8

### Preparation of MNPCs Comprising Charged Block Copolymers and Polyion Complexes

In a simplest way MNPCs comprising charged block copolymers are produced by reacting dispersed MNPs with AB or ABA polyelectrolyte block copolymers that are modified with an anchor group such as alendronate. Such block polyelectrolytes or block ionomers are “doubly hydrophilic” (containing hydrophilic nonionic A and ionic B blocks) and are fully soluble in water. One challenge in this design is that the polyion blocks may also bind to the magnetite particle surface. However, at least in the case of carboxylate-containing block ionomers they desorb from the magnetite particles in >0.1 M NaCl, while anchor groups provide a stable bond with magnetite under the same conditions (Vishwasrao et al., Chem. Materials 28:3024 (2016)). Therefore, upon binding with MNPs, in the presence of increased salt concentration such block ionomers graft to the magnetite surface through the anchor group in the spacer A block. The ionic B block is linked through the spacer and faces the aqueous media forming a hydrophilic polyelectrolyte-containing shell around a single MNP core (in the case of ABA block ionomers a second hydrophilic A block is also exposed to the aqueous environment). The resulting materials can be further reacted with surfactants (cationic or anionic), multivalent ions, or polyions of opposite charge resulting in formation of polyion complexes. At this stage drugs (e.g., cationic doxorubicin, or mitoxantrone), polynucleotides (e.g., DNA or siRNA), or enzymes (e.g., α-chymotrypsin, β-galactosidase, Cu/Zn superoxide dismutase, catalase, etc.) are introduced as charged components. As a result of charge neutralization the polyion B blocks become insoluble and aggregate. The outcomes greatly depend on the structure of the block ionomers (di-vs. triblock) and the grafting density. In some cases (ABA copolymers and/or high grafting density) the aggregation of neutralized B blocks proceeds within the corona of a single MNP. This leads to formation of an “onion” type structures with a single MNP core surrounded by a polyion complex layer similar to multilayer polyelectrolyte complex micelles. In other cases (AB copolymers, lower grafting density) the neutralized B blocks cross-link between different MNPs resulting in formation of swollen networks, which can self-assemble in nano-ferrogels or bulk gels depending on the structure and concentration of the reactants.

The alternative synthesis of MNPCs involves reaction of the MNPs aqueous dispersions with the pre-formed polyion complex micelles or other types of polyion complexes bearing the anchor groups. In this case the polyion complexes are produced in aqueous media by simple mixing the AB, or ABA block ionomers with the oppositely charged molecules (surfactants, polyions, etc.). Such molecules electrostatically bind with the polyion B blocks, which then segregate into the polyion complex cores surrounded by the hydrophilic shell of the A blocks. At this stage using blends of anchor group-containing and anchor group-free block polyelectrolytes one can obtain polyion complexes having different amount of the anchor groups attached to the shell. This is useful to control the reaction of such polyion complexes with MNPs, and modify the dispersion stability and swelling behavior of the resulting MNPCs.

The foregoing is illustrative of the present invention, and is not to be construed as limiting thereof. The invention is defined by the following claims, with equivalents of the claims to be included therein.

1. A magnetic nanoparticle polymer complex (MNPC) comprising a magnetic nanoparticle coated with one or more polymers, such as a block copolymer, such as an amphiphilic block copolymer.

2-3. (canceled)

4. The MNPC of claim 1, wherein the at least one block copolymer comprises a polyacid block.

5. The MNPC of claim 4, wherein the polyacid block is polyacrylic acid or polymethacrylic acid.

6. The MNPC of claim 1, wherein the at least one block copolymer is polyacrylic acid-poloxamer.

7. (canceled)

8. The MNPC of claim 1, wherein the at least one polymer is attached to the nanoparticle by a polyelectrolyte chain, hydrophilic nonionic polymer, or anchoring group, such as by a covalent link.

9. (canceled)

10. The MNPC of claim 8, wherein the polyelectrolyte chain is a polyanion or a polycation.

11. (canceled)

12. The MNPC of claim 8, wherein the hydrophilic nonionic polymer is poly(ethylene oxide), poly(2-methyl-2-oxazoline), poly(2-ethyl-2-oxazoline, or polysarcosine.

13. The MNPC of claim 1, wherein the nanoparticle is hydrophobically modified and non-covalently linked to a hydrophobic block of the at least one block copolymer.

14. The MNPC of claim 1, wherein the nanoparticle is non-covalently linked to a hydrophilic block of the at least one block copolymer.

15. The MNPC of claim 1, wherein the MNPC comprises a micelle formed by hydrophilic and hydrophobic blocks of the at least one block copolymer.

16. (canceled)

17. The MNPC of claim 1, wherein the nanoparticle has a diameter of less than about 50 nm.

18. (canceled)

19. The MNPC of claim 1, wherein the MNPC has a diameter of less than about 100 nm.

20. The MNPC of claim 1, further comprising a therapeutic agent, a contrast agent, or a targeting moiety.

21-22. (canceled)

23. A pharmaceutical composition comprising the MNPC of claim 1 and a pharmaceutically acceptable carrier.

24. A method of increasing cellular uptake of a magnetic nanoparticle (MNP), comprising coating the MNP with one or more polymers, thereby increasing cellular uptake of the MNP relative to a magnetic nanoparticle without the coating.

25-33. (canceled)

34. A method of treating cancer in a subject in need thereof, comprising administering to the subject the MNPC of claim 1, and remotely actuating the MNPC with a magnetic field, thereby treating the cancer.

35-41. (canceled)

42. A method of selectively killing a cancer cell in the presence of non-cancer cells, comprising delivering to the cancer cell and the non-cancer cells the MNPC of claim 1, and remotely actuating the MNPC with a magnetic field, thereby selectively killing the cancer cell.

43-49. (canceled)

50. A method of disrupting the cytoskeleton of a cancer cell, comprising delivering to the cancer cell the MNPC of claim 1, and remotely actuating the MNPC with a magnetic field, thereby disrupting the cytoskeleton of the cancer cell.

51-54. (canceled)

55. A method of obtaining a biomedical image in a subject in need thereof, comprising delivering to the subject the MNPC of claim 1 and detecting the MNPC, thereby obtaining a biomedical image.

56. A method of detecting cancer in a subject in need thereof, comprising delivering to the subject the MNPC of claim 1 and detecting the MNPC, thereby detecting cancer in the subject.
